(19) United States
(12) Patent Application Publication
(10) Pub. No.: US 2015/0336981 A1
(43) Pub. Date: Nov. 26, 2015

(54) MODIFIED POLYPHENOLS AND MODIFIED POLYPHENOL COMPOSITIONS

(71) Applicant: CELLAROUGE PTY LTD, Delaneys Creek, Queensland (AU)

(72) Inventors: Katrina Louise Orchard-Jardine, Samford (AU); Gregory John Jardine, Samford (AU)

(73) Assignee: CELLAROUGE PTY LTD, Delaneys Creek, Queensland (AU)

(21) Appl. No.: 14/440,402

(22) PCT Filed: Nov. 9, 2012
(86) PCT No.: PCT/AU2012/001380

§ 371 Date: May 4, 2015

(30) Foreign Application Priority Data
Nov. 8, 2012 (AU) 2012904880

(51) Int. Cl. C07D 493/22; A23L 1/30; A23F 5/00; A23G 3/36; A61K 31/366; A23F 3/14

(52) U.S. Cl. CPC C07D 493/22; A61K 31/366; A23F 3/14; A23F 5/00; A23G 3/364; A23L 1/3002; A23V 2002/00

(57) ABSTRACT

This invention relates to polyphenolic compounds, in particular ethoxylated polyphenolic compounds and the use of such compounds in disease prevention, disease treatment, and life extension in humans and animals. The invention also relates to methods of preparing ethoxylated polyphenolic compounds, and preparation of compositions comprising the compounds, including dietary supplements and foods. The inventors describe how modified C-glycosidic ellagitannins are safe, palatable and effective at medium and high doses, and produce broad and profound health outcomes in mammals.

### TECHNICAL FIELD

This invention relates to polyphenolic compounds, in particular ethoxylated polyphenolic compounds and the use of such compounds in disease prevention, disease treatment, and life extension in humans and animals. The invention also relates to methods of preparing ethoxylated polyphenolic compounds, and preparation of compositions comprising the compounds, including dietary supplements and foods.

### BACKGROUND ART

The 20th century has been called the “Aspirin Century”, displacing salicin, an anti-inflammatory agent extracted from bark of the willow tree. The human body metabolizes the prodrug, salicin, to the aspirin precursor, salicyclic acid. Salicyclic acid was traditionally used sparingly due to its unpleasant taste and tendency to damage the stomach. Hence, doses of the active ingredient were small, rendering it less effective.

The modification of salicyclic acid by reaction with acetic anhydride produces the less irritable acetylsalicylic acid (aspirin). Aspirin is also combined with caffeine and sold as the analgesic Anacin.

Ever since Bayer's aspirin invention (US patent 1900) natural products have been a very distant second to drugs in the treatment and prevention of disease and aging related illness.

Of the roughly 150,000 people who die each day across the globe, about two thirds-100,000 per day—die of age-related causes. In industrialized nations, the proportion is much higher, reaching 90% (de Grey ADNJ, ‘Life Span Extension Research and Public Debate: Societal Considerations’, Studies in Ethics, Law, and Technology, 2007; 1(1), doi:10.2202/1941-6008.1011).

There has been major progress in the development of pharmaceuticals for disease treatment but there continue to be shortfalls, particularly with side effects and poor compliance. The search in the natural world for competition to potent pharmaceuticals for disease treatment and prevention has been essentially fruitless. Conservative food processers have not provided a challenge to the high-risk pharmaceutical company's capability to produce potent medicines for human and animal health.

Arthritis and diseases of affluence such as cancer, obesity and metabolic syndrome, all have inflammation in common. While genetic factors are considered a factor in some instances, environmental factors, in particular our modern diet rich in processed foods seem to carry the heavy burden of blame.

Cancer is the number one or number two killer in many countries. The weapons against cancer are somewhat cruel and brutal. Tumours are cut out, burnt with radiation, or treated with drugs that poison rapidly dividing cells (chemotherapy). These methods are undeniably effective, but inevitably inflict collateral damage.

Recent developments in targeted cancer treatment, which act on molecular pathways active in many cancer cells, have the advantage of less harsh side effects than are generally associated with chemotherapy. However these successful targeted cancer therapies generally only prolong life by a few months and can pose a considerable financial burden to the consumer or the government that is providing healthcare to its population. Essentially, the high rate of mutation of cancer cells allows them to avoid the relevant molecular pathways.

Current treatment for metabolic syndrome often includes low dose aspirin and a cocktail of pharmaceuticals that target blood pressure, cholesterol, diabetes and obesity. Poor compliance, cost and side effects are major issues in this area of cardiovascular disease.

Arthritis is a name for a group of conditions affecting the joints. There are more than 100 forms of arthritis. In Australia, nearly one in five people has arthritis. The most common forms of arthritis are osteoarthritis, rheumatoid arthritis and gout (see, http://www.betterhealth.vic.gov.au/bhcv2/bhcarticles.nsf/pages/Arthritis).

Treatment is generally effective with long-term pharmaceutical use, but there are adverse side effects.

Despite the obvious need, finding single drugs to treat multiple diseases has been challenging. All biological processes and most diseases have back-up strategies, making drug discovery complex and challenging.

Over the last 20 years, a significant body of evidence has emerged indicating that chemically diverse classes of naturally-occurring substances derived from higher plants are of potential interest for therapeutic interventions in several inflammatory diseases.

Increasingly it is being found that a large range of plant-derived constituents interfere with three relevant targets involved in the inflammatory process, namely arachidonic acid metabolite pathways, nitric oxide and nuclear factor kappa-light-chain-enhancer of activated B cells (NF-κB). This has expanded their potential therapeutic use in the management of relevant inflammatory related diseases (Calixto J B, Otuki M F and Santos A R S, ‘Anti-inflammatory compounds of plant origin. Part I. Action on arachidonic acid pathway, nitric oxide and nuclear factor κ B (NF-κB)’, Planta Med., 2003; 69(11):973-983).

By way of example, many antioxidants found in foods have anti-inflammatory properties, such as those found in tea, coffee, wine and chocolate, yet these anti-inflammatory foods may impact on prolonged inflammation (also referred to as chronic inflammation), but are not potent enough to bring about pain relief in humans. In many instances these antioxidant-rich foods can be consumed in high doses but their bioavailability in humans is poor, hence delivering poor health outcomes.

The family of NF-κB transcription factors is intimately involved in the regulation of expression of numerous genes in the setting of the inflammatory response. Since inflammatory processes play a fundamental role in the damage of articular tissues, many in vitro and in vivo studies have examined the contribution of components of the NF-κB signaling pathways to the pathogenesis of various rheumatic diseases, in particular, of osteoarthritis (OA) and rheumatoid arthritis (RA). Inflammation, cartilage degradation, cell proliferation, angiogenesis and pannus formation are processes in which the role of NF-κB is prominent (Roman-Blas J A and Jimenez S A, ‘NF-κB as a potential therapeutic target in osteoarthritis and rheumatoid arthritis’, Osteoarthritis Cartilage, 2006; 14(9):839-848).

NF-κB is a transcription factor that plays a critical role across many cellular processes including embryonic and neuronal development, cell proliferation, apoptosis, and immune responses to infection and inflammation. Dysregulation of NF-κB signaling is associated with inflammatory diseases and certain cancers. Constitutive activation of NF-κB signaling has been found in some types of tumours including breast, colon, prostate, skin and lymphoid, hence therapeutic blockade of NF-κB signaling in cancer cells provides an attractive strategy for the development of anticancer drugs (Miller S C, Huang R, Sakamuru S, Shukla S J, Attene-Ramos M S, Shinn P, Van Leer D, Leister W, Austin C P and Xia M, ‘Identification of known drugs that act as inhibitors of NF-κB signaling and their mechanism of action’, Biochem. Pharmacol., 2010; 79(9):1272-1280).

The impact that NF-κB has on cancer metastasis, angiogenesis and apoptosis provides a means to its beneficial application for all cancers.

NF-κB transcription factors regulate several important physiological processes, including inflammation and immune responses, cell growth, apoptosis, and the expression of certain viral genes. Therefore, the NF-κB signaling pathway has also provided a focus for pharmacological intervention, primarily in situations of chronic inflammation or in cancer, where the pathway is often constitutively active and plays a key role in the disease. Now that many of the molecular details of the NF-κB pathway are known, it is clear that modulators of this pathway can act at several levels.

Over 750 inhibitors of the NF-κB pathway have been identified, including a variety of natural and synthetic molecules. These compounds include antioxidants, peptides, small RNA/DNA, microbial and viral proteins, small molecules, and engineered dominant-negative or constitutively active polypeptides. Several of these molecules act as general inhibitors of NF-κB induction, whereas others inhibit specific pathways of induction. In addition, some compounds appear to target multiple steps in the NF-κB pathway. Pharmaceutical NF-κB inhibitors (for example, denosumab) are now entering the clinical arena. Moreover, the therapeutic and preventative effects of many natural products may, at least in part, be due to their ability to inhibit NF-κB.

NF-κB is activated by multiple families of viruses, including human immunodeficiency virus 1 (HIV-1), human T-lymphotropic virus 1 (HTLV-1), hepatitis B virus (HBV), hepatitis C virus (HCV), Epstein-Barr virus (EBV), and influenza virus. This activation may serve several function: to promote viral replication, prevent virus-induced apoptosis, and mediate the immune response to the invading pathogen.

As viruses evolve under the highly selective pressures of the immune system, they acquire the capacity to target critical steps in the host cell life, hijacking vital cellular functions to promote viral pathogenesis. Many viruses have evolved mechanisms to target the NF-κB pathway to facilitate their replication, cell survival, and evasion of immune responses. In addition, some viruses use the NF-κB pathway either for its antiapoptotic properties to evade the host defense mechanisms or to trigger apoptosis as a mechanism of virus spread (Hiscott J, Kwon H and Génin P, ‘Hostile takeovers: viral appropriation of the NF-κB pathway’, J. Clin. Invest., 2001; 107(2):143-151, doi:10.1172/JCI11918).

Increasing recognition of the benefits brought about by plant polyphenolics for human health has sparked a new appraisal of various plant-derived food and beverages, such as fruit juices, olive oil, chocolate, coffee, tea and even alcoholic beverages such as wine and cider.

However, excessive consumption of these polyphenolic foods often places a high calorific load on the human diet (for example, fruit juices such as pomegranate juice or cranberry juice), or in the case of caffeinated and alcoholic foods, excess intake results in negative health outcomes.

Plants have been widely used since antiquity as folk medicines. Some of these natural medicines contain the polyphenol antioxidant group, ellagitannins, as the principal “active” constituent.

All ellagitannins contain the ellagic acid moiety:

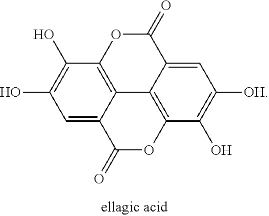 [SMILES: O=c1oc2c(O)c(O)cc3c(=O)oc4c(O)c(O)cc1c4c23]

Ellagic acid is comprised of two gallic acid moieties:

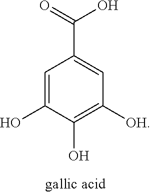 [SMILES: O=C(O)c1cc(O)c(O)c(O)c1]

Plants produce ellagic acid and convert it to a form of tannin known as ellagitannins. These are glucosides that are readily hydrolyzed by water to regenerate ellagic acid when the plants are eaten by mammals.

While it is acknowledged that the ellagitannins do deliver some health benefits, it is widely reported that there is large inter-individual variability due to variability in mammalian gut microflora.

In mammals, the hydrolysis of the ellagic acid group from the parent ellagitannins occurs quite readily. However, it requires subsequent gut microflora activity to metabolize the ellagic acid into the biologically active entity—the urolithins.

Ellagitannins and their hydrolysis product, ellagic acid, are poorly absorbed by mammals. It is generally accepted (although not conclusive) that urolithins, the gut microflora derived metabolites of ellagic acid, are the entities that deliver the health benefits observed with ellagitannins.

By way of example, the gut microbe polyphenol metabolite urolithin-A inhibits the activation of transcription factors like NF-κB and signaling pathways that drive inflammation.

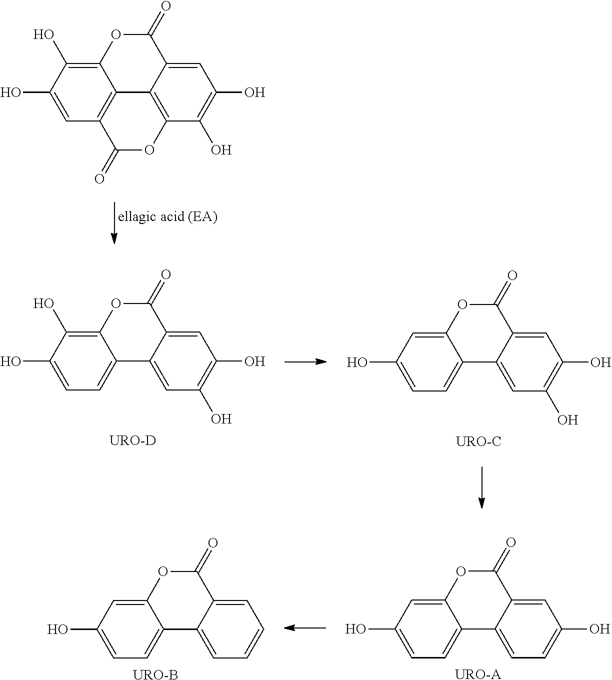

The above diagram shows the ellagic acid metabolites produced by intestinal microflora in animals and humans.

URO-D=Urolithin D (tetrahydroxydibenzopyranone)

URO-C=Urolithin C (trihydroxydibenzopyranone)

URO-A=Urolithin A (dihydroxydibenzopyranone)

URO-B=Urolithin B (monohydroxydibenzopyranone)

A small sub-group of ellagitannins, the C-glycosidic ellagitannins, are found in species of oak (Quercus sp.) and chestnut (Castanea sp.)

These C-glycosidic ellagitannins all contain both the ellagic acid moiety and the tri-gallic acid moiety, typified by vescalagin or its epimer, castalagin.

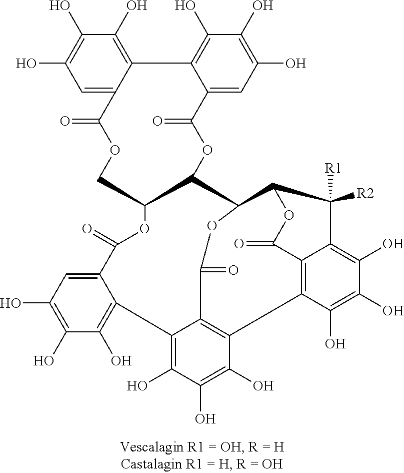

Under strong acidic conditions, such as exist in the mammalian stomach, these C-glycosidic ellagitannins hydrolyse ellagic acid to form adducts with a tri-gallic acid moiety, such as vescalin and castalin.

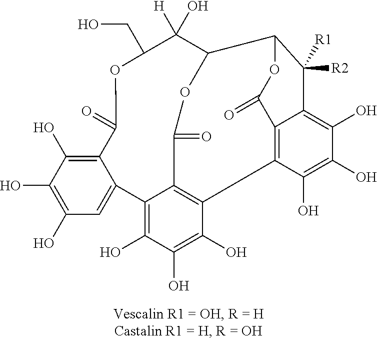

Vescalin and castalin are not found in oak (nor are they naturally occurring anywhere else in nature) but they are formed in small amounts from their respective ellagitannins, vescalagin and castalagin, following thermal treatment (known as ‘toasting’) of oak barrel staves when making oak barrels.

Examples of such vescalin-based oak C-glycosidic ellagitannins also include grandinin, roburin E, roburin A (a vescalagin dimer), roburin D (a castalagin dimer), and the dimers roburin B and roburin C:

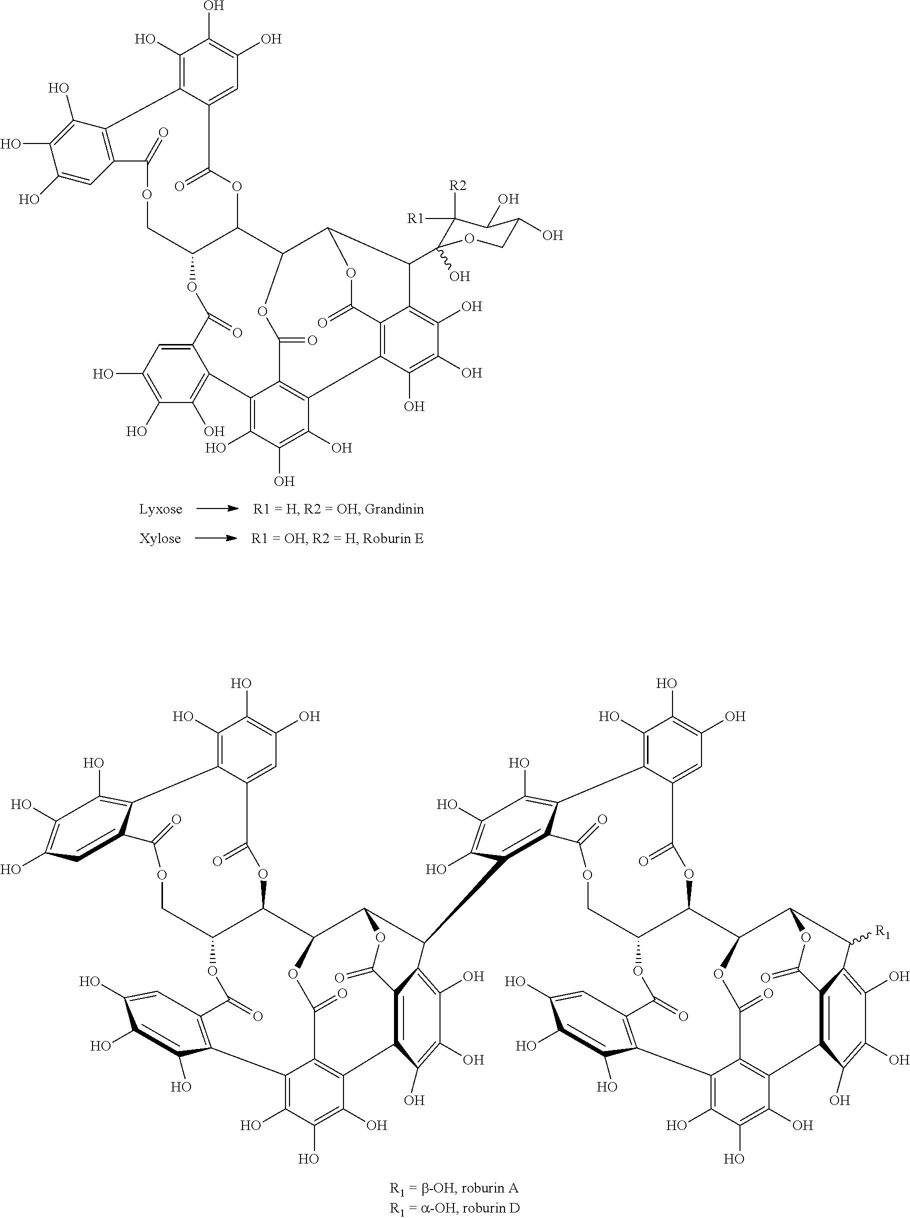

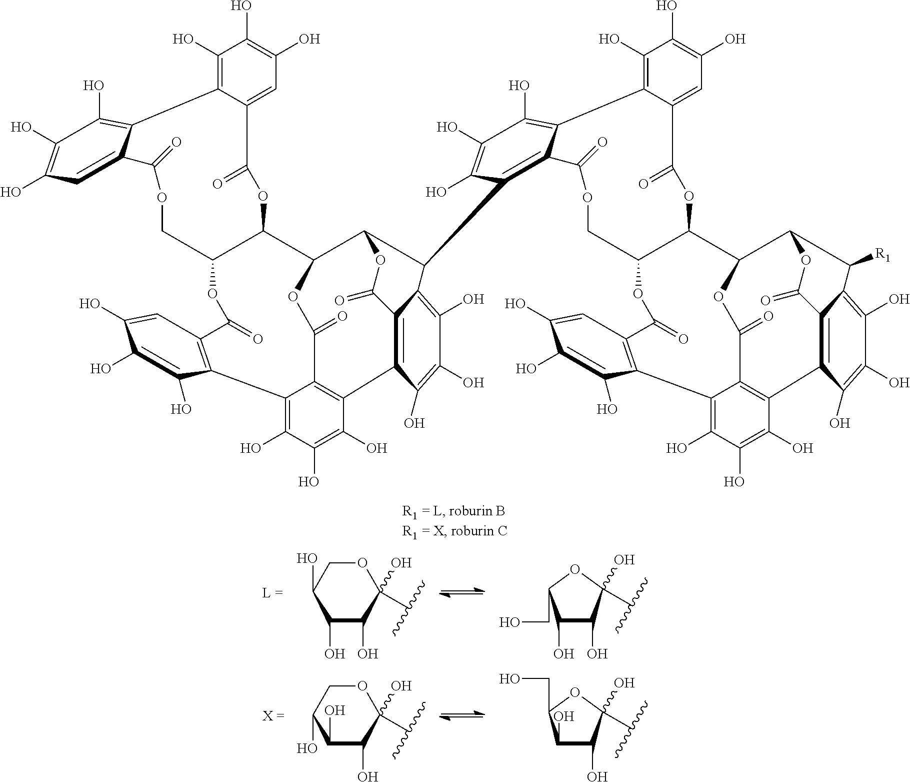

As is the case with other dietary ellagitannins, when an oak-matured beverage such as wine, brandy or whisky is consumed, the human body also converts (hydrolyses) the C-glycosidic ellagitannins into ellagic acid.

Oak has long been the timber of choice for making wine barrels. Although storing wine in oak barrels was probably initially merely for convenience, it was eventually realized that the wine actually improved as a result of storage in oak. During storage, some of the polyphenols in the oak are extracted into the wine, such that oak polyphenols have been consumed by humans, albeit in very low doses, for as long as wine has been stored in oak.

Oak polyphenol compounds, including those depicted above, fall within the category of plant tannins, and are known to have antioxidant properties. Red wine matured in oak contains only very small amounts of oak C-glycosidic ellagitannins, generally about 1 to 2 mg per 150 mL. There are no known food sources that would provide higher doses of oak C-glycosidic ellagitannins. Even if such food sources were known, plant tannins such as the C-glycosidic ellagitannins from oak are not particularly palatable at high doses. Specifically, products containing higher amounts of oak C-glycosidic ellagitannins than is commonly found in red wine, have an astringent, tannic taste that is generally considered unpleasant.

For comparison, when oak is used in winemaking, the levels in the resultant wine are typically quite low—generally a 100 mL portion of oaked red wine would be made with typically 0.1 to 0.2 grams of oak chips.

Therefore, whilst it may be desirable to supplement the mammalian diet with high levels of oak C-glycosidic ellagitannins for the antioxidant activity provided by their urolithin metabolites, and thus the health benefits associated with a diet high in antioxidants such as urolithin metabolites, most mammals would not find the taste profile of any product containing higher amounts of oak C-glycosidic ellagitannins than is commonly found in red wine acceptable.

### DETAILED DESCRIPTION OF THE INVENTION

A general objective of this invention is to provide modified polyphenol compounds and compositions comprising the modified polyphenol compounds that address a range of health issues, including some of the issues as described in the Background Art section, above.

Another general objective of the present invention is to provide the public with a useful, convenient, efficacious, cost-effective or commercial choice.

Another general objective of the present invention is to return natural products such as foods to a position alongside or as an alternative to pharmaceuticals, to be used as a viable treatment or in the prevention of disease.

Another general objective of the present invention is to offer single foods or food cocktails (that is, ‘food.products’) that can be effective and complement and/or replace pharmaceuticals in the disease arena.

Another general objective of the present invention is to offer efficacious, commercial choices for products that are used in addition to conventional medical treatments (e.g. chemotherapy, radiation therapy and hormone therapies) and provide a reduction in side effects (e.g. nausea, alopecia, fatigue, loss of appetite, digestive issues, skin disorders, sexual function).

Another general objective of the present invention is to offer efficacious, cost-effective, convenient and commercial choices for products that are used in addition to conventional medical treatments and provide support to the wellbeing of humans or animals.

Another general objective of the present invention is to offer efficacious, cost-effective, commercial choices for products that are used in addition to conventional medical treatments thus providing enhanced health outcomes in the treatment or prevention of the condition when compared with conventional medical treatment without this invention.

The above-mentioned general objectives (i.e. general advantages that the present invention may provide) may be individually met by one or more embodiments of the present invention as described herein.

The inventors have described herein for the first time that the tri-gallo moiety, not the di-gallo moiety (ellagic acid) described extensively in the literature, is a potent entity for delivery of profound health outcomes in mammals.

The inventors have discovered that ethoxylating C-glycosidic ellagitannins under acidic conditions results in modified C-glycosidic ellagitannins which have an improved taste profile.

In addition, the inventors have surprisingly discovered that modified C-glycosidic ellagitannins are well tolerated (i.e. pallatable) at medium and high doses and produce broad and profound health outcomes in mammals.

The inventors have also discovered that combining modified C-glycosidic ellagitannins with caffeine-rich polyphenolic foods under acidic conditions, results in caffeine-vescalagin adducts that enhance the mammalian health outcomes achieved by the modified C-glycosidic ellagitannins and concomitantly lower caffeine levels in said foods.

The inventors have also discovered that the modified C-glycosidic ellagitannins can be added to foods to create food products that provide a high dietary level of C-glycosidic ellagitannins without the astringent, tannic taste previously associated with high levels of ellagitannins.

Advantageously, food products (including modified food products) comprising the modified C-glycosidic ellagitannins can be used in the prevention and treatment of disease. Food products comprising modified C-glycosidic ellagitannins are particularly useful in the prevention and treatment of diseases linked to NF-κB regulation and nitric oxide deficiency.

The inventors have also discovered that the use of non-ethoxylated C-glycosidic ellagitannins in formulations where the other polyphenolic components are ethoxylated, also produces strong health outcomes when compared with pharmaceuticals.

Compounds and formulations of this invention can result in strong inhibition of NF-κB signaling, thus allowing for an array of profound health outcomes that are found to be impacted by the downstream influence of this master regulator (Gilmore T D and Herscovitch M, ‘Inhibitors of NF-κB signaling: 785 and counting’, Oncogene, 2006; 25:6887-6899).

Diseases that compounds and formulations of this invention can provide viable solutions for include, but are not limited to, aging-related conditions, cancers including metastatic cancers, heart disease, metabolic syndrome, inflammation (both chronic and acute), chronic fatigue, obesity, erectile dysfunction, appetite suppression, sleep disorders, urinary tract infections, asthma, enlarged prostate, excessive alcohol consumption, male pattern baldness and arthritis.

With regards arthritis, this is a major cause of disability and chronic pain in Australia. Standard treatment has been glucocorticoids, disease-modifying anti-rheumatic drugs, non-steroidal anti-inflammatory drugs, such as aspirin and ibuprofen, COX-2 inhibitors such as celecoxib and more recently tumour necrosis factor (TNF)-alpha blockers. Many patients respond poorly and adverse effects are common. Therefore, there is an unmet clinical demand for new anti-arthritic drugs. The ideal anti-arthritic drug to treat chronic inflammation, accompanied by pain, would be a potent, relatively inexpensive, easily administered, stable compound, with analgesic properties and with minimal adverse effects.

Food products (including modified food products) comprising modified C-glycosidic ellagitannins are also useful in the prevention and treatment of diseases linked to nitric oxide deficiency. Some of the known functions of nitric oxide relate to the cardiovascular system, nervous system, lungs, gastrointestinal tract, renal system and immune system.

In particular, the inventors have surprisingly found that modifying C-glycosidic ellagitannins by increasing their lipophilicity results in modified C-glycosidic ellagitannins that have at least the same physiological activity as the corresponding unmodified C-glycosidic ellagitannins. In the context of this invention, the phrase ‘physiological activity’ is intended to be construed broadly to encompass any type of physiological activity. For example, the physiological activity can be antioxidant activity, analgesic, anti-inflammatory activity or anti-tumour activity.

In some embodiments of the invention, the modified C-glycosidic ellagitannins have increased physiological activity when compared to the physiological activity of the corresponding unmodified C-glycosidic ellagitannins. These embodiments are particularly preferred.

Additionally, modified C-glycosidic ellagitannins according to the present invention have an improved taste profile when compared to the taste profile of the corresponding unmodified C-glycosidic ellagitannins. The modified C-glycosidic ellagitannins can thus be used to provide a high dietary level of ellagitannins without the astringent, tannic taste previously associated with high levels of ellagitannins.

The modified C-glycosidic ellagitannins can be provided as a dietary supplement in any suitable form. The modified ellagitannins can, for example, be provided as a supplement in the form of a dried food extract.

Alternatively, the modified ellagitannins can be provided as a supplement in the form of a pharmaceutical composition. The pharmaceutical composition can comprise one or more modified C-glycosidic ellagitannins and a pharmaceutically acceptable carrier or diluent.

Preferably, the pharmaceutical compositions are for oral administration and can be in tablet, capsule, powder or liquid form. A tablet can include a solid carrier such as gelatine or an adjuvant or an inert diluent. Liquid pharmaceutical compositions generally include a liquid carrier suitable for human consumption, such as water, or any other beverage.

In addition to the at least one compound and a carrier or diluent, compositions according to the invention can further include a pharmaceutically acceptable excipient, buffer, stabiliser, isotonicising agent, preservative or anti-oxidant or any other material known to those of skill in the art. It will be appreciated by the person of skill that such materials should be non-toxic and should not interfere with the efficacy of the one or more modified C-glycosidic ellagitannins. With regard to buffers, aqueous compositions typically include such substances so as to maintain the composition at a close to physiological pH or at least within a range of about pH 5.0 to 8.0.

The pharmaceutical compositions of the invention can also be provided in a form suitable for topical application to the skin of a subject. Forms suitable for topical application of the pharmaceutical compositions of the invention include as a solution, cream, ointment, lotion or gel. Compositions for topical application can thus be formulated in a suitable ointment containing the one or more modified C-glycosidic ellagitannins suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Carriers, diluents and excipients which can be used in the pharmaceutical compositions of the invention will be known to those of skill in the art. In addition to those listed above, the British Pharmacopoeia (BP) and the United States Pharmacopeia and National Formulary (USP-NF) contain details of suitable carriers, diluents and excipients, as does Sweetman S (Ed.), ‘Martindale: The complete drug reference.’ London: Pharmaceutical Press, 37^{th} Ed., (2011), and Rowe R C, Sheskey P J, Quinn M E (Ed.), ‘Handbook of Pharmaceutical Excipients’, 6^{th} Ed., London: Pharmaceutical Press (2009), the contents of which are incorporated herein by cross reference.

Alternatively, the pharmaceutically acceptable compositions can be formulated in a suitable lotion or cream containing the one or more modified C-glycosidic ellagitannins suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, glycerol, propylene glycol, isopropyl myristrate, menthol, eucalyptus and water.

An alternative administration regime comprises passing one or more compounds of the invention, such as modified C-glycosidic ellagitannins through an artificial stomach (effectively, a bioreactor) prior to formulating for oral administration or topical application. The artificial stomach processes the modified C-glycosidic ellagitannins so that the output of the artificial stomach is then suitable for administration to subjects who are not able to metabolize the modified C-glycosidic ellagitannins themselves. Subjects who have had a portion of their stomach surgically removed may benefit from such ‘pre-processing’ of the compounds of the invention by an artificial stomach. Subjects with skin cancer, whose skin is challenged and therefore may not necessarily be receptive to direct topical application of modified polyphenol compounds of the invention may also benefit from ‘pre-processing’ of the compounds of the invention by an artificial stomach to reduce molecular size prior to formulation of a composition, thus assisting in the skin absorption required by topical application. The ‘pre-processing’ of the compounds of the invention by an artificial stomach also has application to non-mammalian veterinary medicines (e.g. bird treatment).

Alternative administration regimes could comprise passing one or more compounds of the invention, such as modified C-glycosidic ellagitannins, through nasal, buccal, lingual, sublingual, transdermal or intradermal means.

Preferably, the modified C-glycosidic ellagitannins are provided as a supplement in the form of a fortified food product (i.e. a modified food product). Advantageously, the modified C-glycosidic ellagitannins of the invention can be formulated into a wide variety of food groups or food types. There are presently many different types of commercially available supplemented health food products in the marketplace and many of these can be formulated or modified to incorporate modified C-glycosidic ellagitannins.

Particularly preferred examples of food groups/types that can be fortified by inclusion of modified C-glycosidic ellagitannins of the invention include those for beverages, such as tea leaves and ground coffee. Health bars, such as muesli, fruit and nut bars can also be fortified by inclusion of modified C-glycosidic ellagitannins of the invention. Other examples of food groups/types that can be fortified by inclusion of modified ellagitannins of the invention include confectionary, such as toffee, and baked goods, such as bread and cakes. The modified ellagitannins of the invention can also be used as an additive in commercially-available oaked wines and other alcoholic oaked beverages.

Foods fortified with modified C-glycosidic ellagitannins according to the present invention can further comprise a flavouring agent. Any suitable type and amount of flavouring agent may be used. For example, terpene-based flavours such as lemon oil and lime oil can be used. Other oils such as spearmint and peppermint oil can also be used. Preferably, the oils are produced via a steam-distilled or cold-pressed process prior to use. Alternatively, herb flavours, such as winter tarragon (licorice flavor) or ginger can be used. In a further alternative, artificial flavours such as caramel or cola can be used.

Broadly, the invention concerns modified polyphenolic compounds.

According to a first aspect of the present invention, there is provided a modified polyphenolic compound, wherein a polyphenolic compound has been modified such that the lipophilicity of the modified polyphenolic compound is increased when compared to the lipophilicity of the unmodified polyphenolic compound.

According to a second aspect of the invention, there is provided a composition comprising at least one modified polyphenolic compound of the first aspect.

According to a third aspect of the invention, there is provided a fortified food product, wherein food has been fortified with at least one modified polyphenolic compound of the first aspect to form a fortified food product.

According to a fourth aspect of the present invention, there is provided a modified food product, wherein food has been modified such that a lipophilicity of the formed food product is increased when compared to the lipophilicity of the food.

According to a fifth aspect of the invention, there is provided a method of producing a modified lipophilic polyphenolic compound, the method comprising the step of:
exposing a polyphenolic compound to an acidic solution containing at least one nucleophile such that the at least one nucleophile reacts with the polyphenolic compound to form a modified lipophilic polyphenolic compound.

According to a sixth aspect of the invention, there is provided a method of manufacturing a food product fortified with a modified polyphenolic compound, the method comprising the step of:
combining a modified lipophilic polyphenolic compound with food, thereby fortifying the food and producing a food product.

The method optionally comprises a step of producing a modified lipophilic polyphenolic compound according to the fifth aspect.

According to a seventh aspect of the invention, there is provided a method of manufacturing a modified food product, the method comprising the steps of:

(i) combining a modified lipophilic polyphenolic compound with food; and
(ii) drying the combined modified lipophilic polyphenolic compound and food so as to form a modified food product.

The method optionally comprises a step of producing a modified lipophilic polyphenolic compound according to the fifth aspect.

According to an eighth aspect of the invention, there is provided a method of treating or preventing a medical condition in a subject, the method comprising the step of administering to the subject a therapeutically effective amount of at least one modified polyphenolic compound of the first aspect, a composition of the second aspect, a fortified food product of the third aspect, or a modified food product of the fourth aspect.

According to a ninth aspect of the invention, there is provided use of at least one modified polyphenolic compound of the first aspect in the manufacture of a medicament for the prevention or treatment of a medical condition in a subject.

It is to be appreciated that definitions for other aspects of the present invention may be derived from other statements concerning the invention as found elsewhere within this specification.

Turning to the first aspect, the polyphenolic compound can be any type of polyphenolic compound. In the context of this invention, the term ‘polyphenol’ is to be construed broadly and as such is intended to encompass compounds having two or more phenolic units. The polyphenolic compound can thus be curcumin, shown in its enol and keto forms:

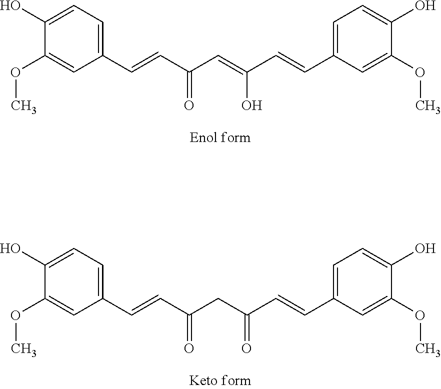 [SMILES: COc1cc(/C=C/C(=O)/C=C(O)/C=C/c2ccc(O)c(OC)c2)ccc1O ; COc1cc(/C=C/C(=O)CC(=O)/C=C/c2ccc(O)c(OC)c2)ccc1O]

An anthocyanin, oenin, from the skin of purple grapes, is a polyphenol responsible for giving colour to red wine:

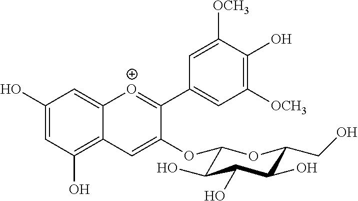

Further examples of polyphenols relevant to the present invention include vescalin and its epimer castalin:

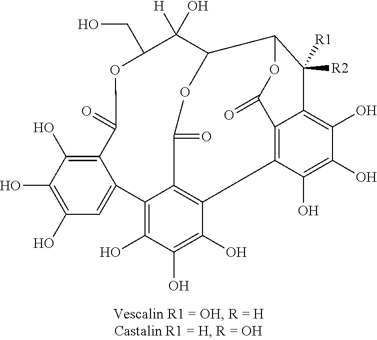

A further example of a polyphenol is a metabolite of an ellagitannin, urolithin A:

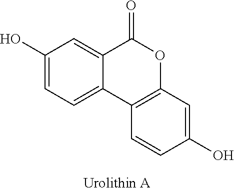 [SMILES: O=c1oc2cc(O)ccc2c2ccc(O)cc12]

Yet further examples include tannic acid, pedunculagin, flavonols (such as quercetin, kaempferole, myricetin, rutin, isorhamnetin), flavanones (such as hesperidin, silibinin, eriodictyol), flavones (such as apigenin, luteolin), flavan-3-ols (such as catechins, theaflavin, thearubigins), anthocyanins (such as pelargonidin, peonidin, cyaniding, delphinidin, malvidin, petunidin) isoflavones (such, as daidzein, genistein, glycitein), hydroxycinnamic acids (such as caffeic acid, chlorogenic acid), oleuropein and piceatannol.

The lipophilicity of the polyphenolic compound can be modified in any suitable way. A preferred method for modifying the lipophilicity of the polyphenolic compounds is to add at least one nucleophile.

As used herein, the term ‘nucleophile’ is intended to have the standard meaning in the art, that being a species that donates an electron-pair to an electrophile to form a chemical bond in a reaction.

The nucleophile can be any suitable nucleophile the addition of which would result in an increase in lipophilicity of the polyphenol. The nucleophile can thus be an alcohol, for example, a C₂-C₈ alcohol, such as ethanol or n-butanol. The nucleophile can also be a polyol, such as glycerol.

The nucleophile can also be a substituted ethanol, such as hydroxytyrosol, which is found in olive leaves and olives:

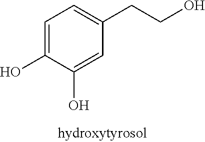 [SMILES: OCCc1ccc(O)c(O)c1]

The nucleophile can be an alkaloid, for example caffeine or nicotine:

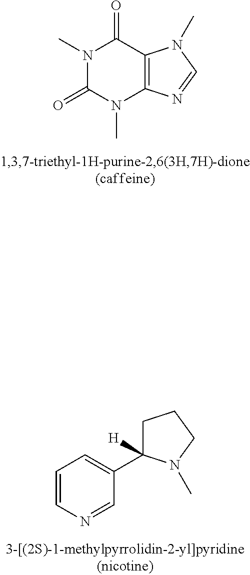

Compounds as diverse as ascorbic acid and polyacetylenes can also act as nucleophiles. The nucleophile can itself be a polyphenol, for example an anthocyanin, or a curcuminoid. Other nucleophiles suitable for increasing the lipophilicity of polyphenols include catechin, itself a polyphenol:

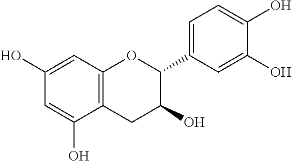 [SMILES: Oc1cc(O)c2c(c1)O[C@H](c1ccc(O)c(O)c1)[C@@H](O)C2]

Alternatively, the modification of polyphenols could be such that the polyphenol is coupled to a pharmaceutical entity, thus providing possibly enhanced efficacy or a means for delivery/administration of both active constituents.

Preferably, the modification of polyphenols by the addition of at least one nucleophile to produce a compound with increased lipophilicity, is undertaken under acidic conditions. It is particularly preferred that the nucleophilic addition is undertaken within the pH range of about pH 3 to about pH 4.5. The modification of a polyphenol to form a compound having increased lipophilicity is thus preferably undertaken by acid-catalyzed nucleophilic substitution. The general reaction schemes for modification of oak ellagitannins such as vescalagin (acid-catalyzed nucleophilic substitution) and castalagin (acid-catalyzed Michael addition) are as follows:

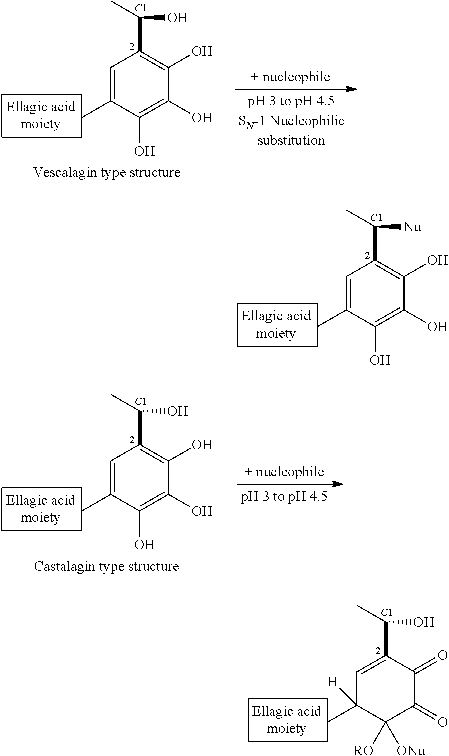

A specific example of such a reaction is the ethoxylation of vescalagin or castalagin, which can result in the addition of either one or two ethoxy groups to the 1-galloyl moiety:

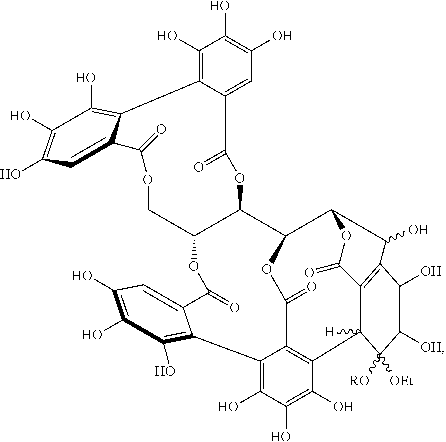

where R=H or Et

Alternatively, the modification of polyphenols by the addition of at least one nucleophile to produce a compound with increased lipophilicity, is undertaken under non-acidic conditions such as pulsed electric field processing conditions or high salt environment. The modified polyphenolic compound can be in a substantially isolated, purified, pure, enriched, concentrated, extracted or homogeneous state, for example. That is, the modified polyphenolic compound can be an isolate, a concentrate or extract, etc.

The modified polyphenolic compound can be in the form of a pro-drug, requiring in vivo and/or in vitro processing into a more physiologically active form.

According to a preferred embodiment, foods fortified with modified polyphenolic compounds according to the present invention comprise an amount of modified polyphenol sufficient to provide per day, the equivalent amount of modified polyphenols that would be obtained from about 1 g to about 12 g of oak chips. Foods fortified, with modified polyphenolic compounds according to the present invention can therefore comprise an amount of modified polyphenol sufficient to provide per day, the equivalent amount of modified polyphenols that would be obtained from about 1 g, 2 g, 3 g, 4 g, 5 g, 6 g, 7 g, 8 g, 9 g, 10 g, 11 g or 12 g of oak chips. The food can be fortified to provide the modified polyphenol in one or more doses. For example, where the fortified food is a beverage such as tea, and the tea is fortified with modified polyphenols to provide per serve, the equivalent amount of modified polyphenols that would be obtained from about 4 g of oak chips, up to three serves/day can be consumed.

Preferably the food is fortified to provide per day, in at least one dose, the equivalent amount of modified polyphenols that would be obtained from about 8 g of oak chips. It is particularly preferred that the food is fortified to provide the equivalent amount of modified polyphenols that would be obtained from about 4 g of oak chips in two serves/day.

The inventors have also advantageously discovered that modification of a food can also be achieved to form adducts which provide beneficial mammalian health outcomes. Formation of such adducts results in a food product that provides enhanced mammalian health outcomes similarly to the enhancement achieved by the modified polyphenolic compounds of the invention.

Specifically, a food can be modified such that the lipophilicity of the food product is increased when compared to the lipophilicity of the unmodified food. Preferably, the food to be modified is intended for use as a beverage. Particularly preferred foods for modification to increase their lipophilicity are tea leaves or ground coffee.

Preferably, the modified food product is also fortified with modified polyphenolic compounds according to the present invention. According to a preferred embodiment, modified food products which are additionally fortified with modified polyphenolic compounds according to the present invention comprise an amount of modified polyphenol sufficient to provide per day, the equivalent amount of modified polyphenols that would be obtained from about 1 to 2 g of oak chips. Thus, where the food product is also modified, the amount of modified polyphenol required to achieve enhanced health outcomes is reduced compared to the amount required when the food product is unmodified.

Any type of polyphenolic compound can be used in the preparation of modified polyphenolic compounds according to the fifth aspect of the invention. As detailed previously, the term ‘polyphenol’ is intended to be construed broadly, and as such, encompasses any compound having more than one phenol moiety. It is preferred that the polyphenol compounds are natural products, such as ellagitannins, curcumins and anthocyanins, however, synthetic polyphenols can also be modified by the methods of the invention to provide modified polyphenolic compounds which will provide enhanced mammalian health outcomes.

Modification of the polyphenolic compound by at least one nucleophile results in a modified polyphenol having increased lipophilicity when compared to the lipophilicity of the unmodified polyphenol. The nucleophile can thus be any suitable nucleophile. Preferably, the nucleophile is capable of undergoing acid-catalysed nucleophilic substitution or acid-catalysed Michael addition with the polyphenol.

As detailed, above, suitable nucleophiles include alcohols, preferably C₂-C₈ alcohols, such as ethanol and n-butanol but can also include substituted alcohols, such as hydroxytyrosol. The nucleophile can also be a polyol, such as glycerol. Alkaloids, such as caffeine and nicotine can behave as nucleophile, as can ascorbic acid and polyacetylenes.

In addition, polyphenols themselves can also behave as nucleophiles in acid-catalysed nucleophilic substitution or acid-catalysed Michael addition to a polyphenol that is the same or different. For example, in one embodiment, the polyphenol may be an anthocyanin and the nucleophile may also be an anthocyanin, resulting in essentially an anthocyanin dimer. As an example of an alternative embodiment, the polyphenol may be an anthocyanin and the nucleophile may be a curcuminoid, resulting in an anthocyanin-curcuminoid adduct.

Preferably, the acid-catalysed reaction takes place in an acidic solution having a pH within the range of about pH 3 to about pH 4.5.

The acid-catalysed reaction is allowed to proceed for a period of time sufficient for the one or more nucleophiles to react with the polyphenolic compound. Typically, the reaction can be allowed to proceed for about 2 days up to about 60 days. The reaction can therefore be allowed to proceed for about 2, 3, 5, 10, 15, 20, 25, 30, 35, 40, 45, 50, 55 or 60 days. Preferably, the reaction is allowed to proceed for about 21 days, after which time almost all of the polyphenol has been converted to modified polyphenol and therefore has increased lipophilicity.

Following completion of the acid-catalysed nucleophilic substitution or acid-catalysed nucleophilic addition, the reaction mixture can be filtered or otherwise separated into its different components to remove any solid material. The solid material can be precipitate that has formed during the reaction. In some embodiments, where oak chips are used as a source of polyphenol (C-glycosidic ellagtitannins), the oak chips are removed by filtration. Any suitable means of filtration known in the art can be utilized to filter the reaction mixture.

The resultant filtrate, which contains the modified polyphenolic compound can then be evaporated using any suitable means known in the art. The filtrate can be evaporated to dryness. Preferably, the filtrate is evaporated, but not to dryness, resulting in a concentrated solution of the modified polyphenolic compound. The filtrate can be evaporated to any desired level of concentration of the modified polyphenolic compound.

The concentrate of the modified polyphenolic compound can be added to food to produce a fortified food in accordance with the sixth aspect of the invention. The concentrate can be added to any type of food to fortify that food. Particularly preferred examples of food that can be fortified according to the invention are tea leaves and ground coffee.

For example, the concentrate can be added to tea leaves to form a slurry. Following stirring of the slurry for about two to about three days, the slurry is then evaporated to dryness. The evaporation can be achieved using any suitable means known in the art. Preferably the evaporation to dryness is undertaken relatively slowly, over a period of two to three days.

The concentrate can also be used to modify a food product in accordance with the seventh aspect of the invention. For example, the concentrate can be added to tea leaves to form a slurry. Following stirring of the slurry for about 2 days to about 3 days, the slurry is then evaporated to dryness. The evaporation can be achieved using any suitable means known in the art. Preferably, the evaporation is undertaken on a drying bed with simultaneous flow of air to assist in drying the tea leaves. The resultant dry tea leaves are not only fortified with modified polyphenol but components of the tea leaves are also modified.

Compositions or food products of the invention can be used to prevent or treat any suitable type of medical condition, aging related conditions or to provide for a longer, healthier and happier life.

For example, the compositions or food products described in this invention could be used for acute inflammation, abdominal fat reduction, aging related conditions, aggression associated with dementia, alcohol consumption reduction, anti-inflammatory, alopecia, alopecia associated with chemotherapy, alopecia associated with Taxol™, anti-depressant, anxiety, Alzheimer's disease, appetite suppression, arthritis, asthma, antioxidant deficiency, acne, alcoholic liver disease, appetite suppression, avian influenza (NF-κB), blood pressure (high), bronchial pneumonia recovery, body “fullness reduction”, benign prostate hyperplasia, Barrett's esophagus, benign skin tumours, bowel motion issues, cancer, cancer (early stage), cancer (metastatic), cancer re-occurrence, cholesterol, chronic fatigue syndrome, chemotherapy side effect reduction, caffeine dependence reduction, chemotherapy reduction, chemotherapy replacement, coffee consumption reduction, calming, chest infection recovery, carpal tunnel syndrome, compliance (poor), colds recovery, cold preventative, cold sores, chemotherapy “flu like symptoms” side effect prevention, chronic inflammation, cardiovascular disease, chronic inflammation management, compromised immune system, circulatory system diseases, cerebral malaria (NF-κB), cysts, constipation, digestion, dietary food for terminally ill, dietary food for chemotherapy patients, dementia, dementia-related behavior, diabetes mellitus, depression, digestive diseases, dry skin, exercise induced muscular stiffness, enlarged spleen pain, energy, erectile function, euphoria, eyesight improvement, eczema, enlarged prostate symptoms, erectile dysfunction, fertility, fatty liver, fingernail damage due to chemotherapy, flu recovery, frozen shoulder, gout, gluten intolerance, gall stones, genital herpes, genital warts, hormone treatment side effect reduction, hospitalisation reduction, hypertension, hair re-growth, heart disease, HIV, (NF-κB), hyperlipidemia, hangover treatment, nitric oxide deficiency diseases, headaches, insomnia, iritis, immune enhancement, infections, immunodeficiency disorders, indigestion, jubilation, joint pain, joint swelling, joint mobility, kidney disease, life extension, libido enhancement/improvement, liver enzymes normalized, liver cysts, lung inflammation, liver disease, metabolic syndrome treatment, metabolic syndrome prevention, mental acuity, mental clarity, mood enhancement, menstruation pain reduction, menstruation breast discomfort reduction, macular degeneration, motivation for exercise, medication reduction, medication replacement, mobility improvement, memory performance, migraine prevention, male pattern baldness (PGD2 downstream of NF-κB), mental impairment, menopausal symptoms reduction, memory loss, mouth ulcers, mouth hygiene, NF-κB disorders, nitric oxide disorders, osteoarthritis, obesity, pain treatment, pain prevention, post traumatic stress disorder treatment, pain relief from bone cancer, pain relief, osteoarthritis, platelet count (B cell lymphoma), penicillin resistant infections recovery, pharmaceutical replacement, pain due to injury, prevention of weight gain, prevention of weight gain following breast cancer treatment, radiotherapy side effect reduction, relaxant, rheumatoid arthritis, skin improvement, stimulant, spleen pain (B cell lymphoma), scrotum sac tightening, sleep improvement, stool softening, stress, somatic disorders, skin healing, spondylosis pain relief, sexual dysfunction, sarcoidosis, swollen lympth glands, urinary tract infections, urine pH normalization, urea abnormalities, uric acid disorders, vaginal lubrication, vaginal dryness, vision problems, weight loss, well tolerated with warfarin medication, wellness and work endurance improvement.

Other non-limiting aspects and embodiments of the invention will become apparent from the following detailed description thereof.

Preferred embodiments of the invention as defined herein are as follows.

In a first embodiment, the invention provides a modified polyphenolic compound, wherein a polyphenolic compound has been modified such that the lipophilicity of the modified polyphenolic compound is increased when compared to the lipophilicity of the unmodified polyphenolic compound.

Preferably, the modified polyphenolic compound is a modified C-glycosidic ellagitannin, a modified ellagitannin, a modified curcumin, a modified anthocyanin, a modified urolithin, a modified roburin, a modified tannin or a modified flavan-3-ol.

More preferably, the modification to the polyphenolic compound is ethoxylation, such that the modified polyphenolic compound is an ethoxylated C-glycosidic ellagitannin, an ethoxylated ellagitannin, an ethoxylated curcumin, an ethoxylated anthocyanin, an ethoxylated urolithin, an ethoxylated roburin, an ethoxylated tannin or an ethoxylated flavan-3-ol.

It is particularly preferred that the modified polyphenolic compound is ethoxylated vescalagin or ethoxylated castalagin.

In a second embodiment, the invention provides a modified polyphenolic compound selected from the group consisting of modified C-glycosidic ellagitannins, modified ellagitannins, modified curcumins, modified anthocyanins, modified urolithins, modified roburins, modified tannins and modified flavan-3-ols.

Preferably, the modification is ethoxylation such that the modified polyphenolic compound is an ethoxylated C-glycosidic ellagitannin, an ethoxylated ellagitannin, an ethoxylated curcumin, an ethoxylated anthocyanin, an ethoxylated urolithin, an ethoxylated roburin, an ethoxylated tannin or an ethoxylated flavan-3-ol.

More preferably, the modified polyphenolic compound is ethoxylated vescalagin or ethoxylated castalagin.

In a third embodiment, the invention provides a composition comprising at least one modified polyphenolic compound according to the first embodiment.

Preferably, the composition is a pharmaceutical composition, a fortified food product, a food product, a modified food product, a fortified beverage or a beverage.

More preferably, the composition is a pharmaceutical composition, a food product or a modified food product.

In particularly preferred embodiments, the composition is fortified ground coffee, fortified black tea leaves, fortified green tea leaves or fortified red wine.

Preferably, the fortification comprises modification of the food product or beverage components. More preferably the modification of the food product or beverage component is ethoxylation.

Preferably, the at least one modified polyphenolic compound is present in the composition in an amount of at least 0.5% (w/w).

The composition can comprise 1, 2, 3, 4, 5, 6, 7, 8, 9, 10, 11, 12, 13, 14, 15, 16 or 17 modified polyphenolic compounds.

Preferably, the composition comprises 1, 2, 3, 4, 5 or 6 modified polyphenolic compounds.

In particularly preferred embodiments, the composition comprises two modified polyphenolic compounds.

In a fourth embodiment, the invention provides a pharmaceutical composition comprising at least one modified polyphenolic compound according to the first embodiment together with a pharmaceutically acceptable carrier or diluent.

Preferably, the at least one modified polyphenolic compound is present in an amount of at least about 0.5% (w/w).

The pharmaceutical composition can comprise 1, 2, 3, 4, 5, 6, 7, 8, 9, 10, 11, 12, 13, 14, 15, 16 or 17 modified polyphenolic compounds.

In particularly preferred embodiments, the pharmaceutical composition comprises two modified polyphenolic compounds.

In a fifth embodiment, the invention provides a food product comprising at least one modified polyphenolic compound of the first embodiment.

Preferably, the at least one modified polyphenolic compound is present in an amount of at least 0.5% (w/w).

The food product of the fifth embodiment can comprise 1, 2, 3, 4, 5, 6, 7, 8, 9, 10, 11, 12, 13, 14, 15, 16 or 17 modified polyphenolic compounds.

In particularly preferred embodiments, the food product comprises two modified polyphenolic compounds.

The food product can be any type of food product, but particularly preferred are beverages, health bars, confectionary and baked goods.

In a sixth embodiment, the invention provides a fortified food product, wherein food has been fortified with at least one modified polyphenolic compound according to the first embodiment to form the fortified food product:

The food product can be fortified with 1, 2, 3, 4, 5, 6, 7, 8, 9, 10, 11, 12, 13, 4, 15, 16 or 17 modified polyphenolic compounds.

In particularly preferred embodiments, the food product is fortified with one modified polyphenolic compound.

In a seventh embodiment, the invention provides a modified food product, wherein food has been modified such that a lipophilicity of the formed food product is increased when compared to the lipophilicity of the food.

The food can be a solid food or a beverage. Preferably the food is ground coffee, black tea leaves, green tea leaves or red wine.

In an eighth embodiment, the invention provides a method of producing a modified lipophilic polyphenolic compound, the method comprising the step of
exposing a polyphenolic compound to an acidic solution containing at least one nucleophile to form a reaction mixture such that the at least one nucleophile reacts with the polyphenolic compound in the reaction mixture to form a modified lipophilic polyphenolic compound.

Preferably, the polyphenolic compound is a C-glycosidic ellagitannin, an ellagitannin, a curcumin, an anthocyanin, a urolithin, a roburin, a tannin or a flavan-3-ol.

Preferably, the acidic solution has a pH of 3.5, 3.6, 3.7, 3.8, 3.9, 4.0, 4.1, 4.2, 4.3, 4.4, 4.5, 4.6, 4.7, 4.8, 4.9 or 5.0. In particularly preferred embodiments, the acidic solution has a pH between 4.2 and 4.5.

The at least one nucleophile is preferably selected from the group consisting of C₂-C₈ alcohols, substituted C₂-C₈ alcohol, polyols, substituted polyols, alkaloids, polyacetylenes, ascorbic acid and polyphenols.

Preferably, the modification is ethoxylation, such that the modified lipophilic polyphenolic compound is an ethoxylated C-glycosidic ellagitannin, an ethoxylated ellagitannin, an ethoxylated curcumin, an ethoxylated anthocyanin, an ethoxylated urolithin, an ethoxylated roburin, an ethoxylated tannin or an ethoxylated flavan-3-ol.

In a particularly preferred embodiment, the modified lipophilic polyphenolic compound is ethoxylated vescalagin or ethoxylated castalagin.

Optionally, the method can further comprise the step of isolating the modified lipophilic polyphenolic compound.

The step of isolating can be undertaken by any suitable means known in the art. Such means can include chromatography, including column chromatography, flash column chromatography, liquid chromatography (LC) or high performance liquid chromatography (HPLC). The step of isolating can also be undertaken using fractionation.

Alternatively, the method can optionally further comprise evaporation of the reaction mixture to obtain a concentrate comprising the modified lipophilic polyphenolic compound.

In a ninth embodiment, the invention provides a method of manufacturing a food product comprising the step of:
combining at least one modified lipophilic polyphenolic compound according to the first embodiment with food, to thereby form the food product.

The food product is preferably selected from the group consisting of beverages, health bars, confectionary and baked goods. In particularly preferred embodiments, the food product is coffee, black tea or green tea.

Preferably, the step of combining at least one modified lipophilic polyphenolic compound with food comprises the steps of:
(i) addition of a solution comprising the at least one modified lipophilic polyphenolic compound to ground coffee, black tea leaves or green tea leaves to form a slurry;
(ii) mixing the slurry for a period of 2 to 3 days; and
(iii) drying the slurry.

The food product can be combined with 1, 2, 3, 4, 5, 6, 7, 8, 9, 10, 11, 12, 13, 14, 15, 16 or 17 modified polyphenolic compounds. In particularly preferred embodiments, the food product is combined with one modified polyphenolic compound.

The drying can be undertaken using any suitable means known in the art. Preferred methods for drying the slurry include evaporation to dryness under ambient conditions or using heat to assist with the evaporation, a refractance window drying process (RWDP), which uses mild heating conditions, or drying on a drying bed under ambient or heated conditions.

In a tenth embodiment, the invention provides a method of manufacturing a modified food product, the method comprising the steps of:
(i) combining at least one modified lipophilic polyphenolic compound according to the first embodiment, or when produced by the method according to the sixth embodiment, with food such that the food is saturated with the at least one modified lipophilic polyphenolic compound; and
(ii) drying the combined at least one modified lipophilic polyphenolic compound and food so as to form a modified food product.

The food can be combined with 1, 2, 3, 4, 5, 6, 7, 8, 9, 10, 11, 12, 13, 14, 15, 16 or 17 modified polyphenolic compounds. In particularly preferred embodiments, the food is combined with one modified polyphenolic compound.

The food can be a solid food or a beverage. Preferably the food is ground coffee, black tea leaves, green tea leaves or red wine.

The drying can be undertaken using any suitable means known in the art. Preferred methods for drying the slurry include evaporation to dryness under ambient conditions or using heat to assist with the evaporation, RWDP, which uses mild heating conditions, or drying on a drying bed under ambient or heated conditions.

In an eleventh embodiment, the invention provides a method of treating or preventing a medical condition in a subject, the method comprising the step of administering to the subject a therapeutically effective amount of a modified polyphenolic compound of the first embodiment, a composition of the second embodiment or a food product of the fifth embodiment.

Preferably, the medical condition is an aging-related condition, cancer including metastatic cancer, heart disease, metabolic syndrome, chronic inflammation, acute inflammation, chronic fatigue, obesity, erectile dysfunction, appetite suppression, a sleep disorder, a urinary tract infection, asthma, enlarged prostate, excessive alcohol consumption, male pattern baldness or arthritis.

More preferably, the medical condition is cancer including metastatic cancer, metabolic syndrome, chronic inflammation, acute inflammation, erectile dysfunction or arthritis.

In a particularly preferred embodiment, the medical condition is metabolic syndrome.

In a twelfth embodiment, the invention provides use of a modified polyphenolic compound of the first embodiment in the manufacture of a medicament for the prevention or treatment of a medical condition in a subject.

Preferably, the medical condition is an aging-related condition, cancer including metastatic cancer, heart disease, metabolic syndrome, chronic inflammation, acute inflammation, chronic fatigue, obesity, erectile dysfunction, appetite suppression, a sleep disorder, a urinary tract infection, asthma, enlarged prostate, excessive alcohol consumption, male pattern baldness or arthritis.

More preferably, the medical condition is cancer including metastatic cancer, metabolic syndrome, chronic inflammation, acute inflammation, erectile dysfunction or arthritis.

In a particularly preferred embodiment, the medical condition is metabolic syndrome.

### BRIEF DESCRIPTION OF THE DRAWINGS

FIG. 1 is a schematic illustrating pathways relevant to the present invention.

FIG. 2 illustrates graphically the effects of ethoxylated vescalagin/castalagin rich extract (EVC) on oral glucose tolerance (A) and systolic blood pressure (B).

FIG. 3 illustrates the effects of EVC on inflammation and fibrosis in the heart induced by high-fat diet (H) feeding.

FIG. 4 illustrates graphically the effects of EVC on vascular responses in rats.

FIG. 5 illustrates the effects of EVC on fat deposition, inflammation and fibrosis in rat livers.

FIG. 6 illustrates the effects of EVC on expression of Nrf2 and NF-κB in the heart and the liver.

FIG. 7 illustrates graphically the effects of EVC on systolic blood pressure, vascular contraction by noradrenaline, vascular relaxation by acetylcholine, and vascular relaxation by sodium nitroprusside in spontaneously hypertensive rats (SHR) and SHR supplemented with EVC.

FIG. 8 illustrates graphically the knee gait and knee joint widths for EVC black tea treated rats and untreated controls.

FIG. 9 illustrates graphically the knee gait and knee joint widths for EVC coffee treated rats and untreated controls.

FIG. 10 illustrates graphically the effects of EVC CAT tarragon tea, PLA2 or ibuprofen on systolic blood pressure of rats fed a high carbohydrate, high fat diet.

FIG. 11 illustrates graphically the effects of EVC CAT tarragon tea, PLA2 or ibuprofen on fat pad measurements of rats fed a high carbohydrate, high fat diet.

FIG. 12 illustrates graphically the effects of EVC CAT tarragon tea, PLA2 or ibuprofen on glucose tolerance of rats fed a high carbohydrate, high fat diet.

FIG. 13 illustrates graphically the knee gait for EVC PC treated rats and untreated controls.

FIG. 14 illustrates graphically the knee joint widths for EVC PC treated rats and untreated controls.

FIG. 15 illustrates graphically the expression of RANKL and OPG in UMR 106 osteoblasts.

FIG. 16 illustrates graphically the knee gait for EVC purple carrot and elderberry treated rats and untreated controls.

FIG. 17 illustrates graphically the knee joint widths for EVC purple carrot and elderberry treated rats and untreated controls.

FIG. 18 illustrates graphically the knee gait for EVC purple carrot and camu camu treated rats and untreated controls.

FIG. 19 illustrates graphically the knee joint widths for EVC purple carrot and camu camu treated rats and untreated controls.

FIG. 20 illustrates graphically (A) knee gait and (B) knee joint widths for EVC PC fucoidan treated rats.

FIG. 21 illustrates the effect of EVC PC fucoidan on the total nucleated cell count in bronchial/alveolar lavage (BAL) fluid.

FIG. 22 illustrates the effect of EVC PC fucoidan on the neutrophil count in BAL fluid.

FIG. 23 compares the BAL from a mouse fed EVC PC fucoidan and the BAL from an LPS positive control mouse.

FIG. 24 illustrates the effect of EVC PC fucoidan on alveolar macrophages in BAL fluid.

FIG. 25 illustrates the effect of EVC PC fucoidan on protein content in BAL fluid.

FIG. 26 illustrates the effect of EVC PC fucoidan on TNFα concentration in BAL fluid.

FIG. 27 illustrates graphically (A) feed intake and (B) water intake for rats on cornstarch (CS, control), HCHF or HCHF+PCGTOIL diets.

FIG. 28 illustrates graphically (A) body weight, (B) % body weight gain, (C) abdominal circumference, and (D) abdominal fat deposition in rats on cornstarch (CS, control), HCHF or HCHF+PCGTOIL diets.

FIG. 29 illustrates graphically (A) left ventricular weight, (B) left ventricular stiffness, (C) systolic blood pressure (SBP), and (D) oral glucose tolerance in rats on cornstarch (CS, control), HCHF or HCHF+PCGTOIL diets.

FIG. 30 illustrates graphically (A) body weight, (B) % body weight gain, (C) abdominal circumference, and (D) abdominal fat deposition in rats on CS, HCHF, HCHF+EVC PC fucoidan and HCHF+lemon lime tea+EVC PC fucoidan diets.

FIG. 31 illustrates graphically (A) left ventricular weight, (B) left ventricular stiffness, (C) systolic blood pressure at 16 weeks, and (D) oral glucose tolerance in rats on CS, HCHF, HCHF+EVC PC fucoidan and HCHF+lemon lime tea+EVC PC fucoidan diets.

FIG. 32 illustrates the effect of EVC CAT wine tea extract on the PC-3 prostate cancer cell line.

FIG. 33 illustrates the effect of EVC CAT wine tea extract, seaweed extract and combined extracts of EVC CAT wine tea and seaweed on the PC-3 prostate cancer cell line.

FIG. 34 illustrates the effect of EVC CAT wine tea extract, seaweed extract and combined extracts of EVC CAT wine tea and seaweed on non-tumourigenic prostate cells.

FIG. 35 illustrates the effect of EVC-PCBC on the non-synchronised LNCaP prostate cancer cell line.

FIG. 36 illustrates the effect of EVC-PCBC on the non-synchronised MDA-PCa-2b prostate cancer cell line.

FIG. 37 illustrates the effect of EVC-PCBC on the non-synchronised MDA-MB-231 breast cancer cell line.

FIG. 38 illustrates the effect of EVC-PCBC treatment on LNCaP prostate cancer cells following serum withdrawal.

FIG. 39 illustrates the effect of EVC-PCBC treatment on PC-3 prostate cancer cells following contact inhibition.4

FIG. 40 illustrates the effect of EVC food extract on the J82 bladder cancer cell line.

FIG. 41 illustrates the effect of EVC food extract on the HT 1376 bladder cancer cell line.

FIG. 42 illustrates the effect of EVC food extract on the AGS stomach cancer cell line.

FIG. 43 illustrates the effect of EVC food extract on the MDA-MB-468 breast cancer cell line.

FIG. 44 illustrates the effect of EVC food extract on the MDA-MB-231 breast cancer cell line.

FIG. 45 illustrates the effect of EVC food extract on the LS180 colon cancer cell line.

FIG. 46 illustrates the effect of EVC food extract on the DLD-1 colon cancer cell line.

FIG. 47 illustrates the effect of EVC food extract on the PC-3 prostate cancer cell line.

FIG. 48 illustrates the effect of EVC food extract on the androgen insensitive LNCaP (AI-LNCaP) prostate cancer cell line.

FIG. 49 illustrates the effect of EVC food extract on the LNCaP prostate cancer cell line.

FIG. 50 illustrates the effect of EVC-EFOOD TEA treatment on Pane 5.04 pancreatic cancer cells.

FIG. 51 illustrates the effect of EVC-EFOOD TEA treatment on LNCaP prostate cancer cells.

FIG. 52 illustrates the effect of EVC-EFOOD TEA treatment on PC-3 prostate cancer cells.

FIG. 53 illustrates the effect of EVC-EFOOD TEA treatment on DU145 prostate cancer cells following contact inhibition.

FIG. 54 compares the effect of (A) EVC food extract/CS and (B) EVC food extract on the LNCaP prostate cancer cell line.

FIG. 55 compares the effect of (A) EVC food extract/CS and (B) EVC food extract on the LNCaP prostate cancer cell line.

FIG. 56 compares the effect of (A) EVC food extract/CS and (B) EVC food extract on the PC3 prostate cancer cell line.

FIG. 57 shows the effect of EVC blueberry juice blend on DNA synthesis.

FIG. 58 shows the effect of EVC blueberry juice blend (EVCB) on the expression of cell cycle regulatory proteins.

FIG. 59 illustrates the effect of EVC blueberry juice blend on the expression of pre-replicative complex proteins.

In order that the invention may be more readily understood and put into practice, one or more preferred embodiments thereof will now be described, by way of example only.

### BEST MODE FOR CARRYING OUT THE INVENTION

As mentioned above, the inventors have discovered that ethoxylating C-glycosidic ellagitannins under acidic conditions results in modified C-glycosidic ellagitannins which have an improved taste profile, the modified C-glycosidic ellagitannins produce broad and profound health outcomes in mammals, and food products comprising the modified C-glycosidic ellagitannins are particularly useful in the prevention and treatment of diseases linked to NF-κB regulation and nitric oxide deficiency.

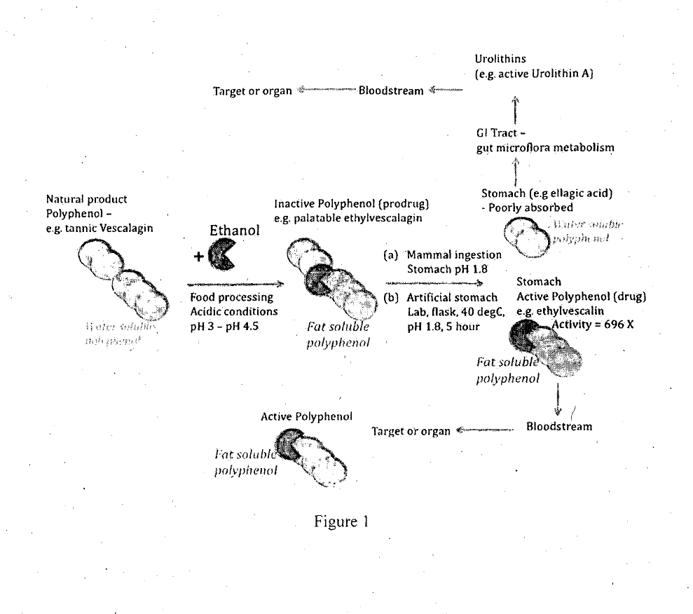

Physiological pathways relevant to the present invention are depicted schematically in FIG. 1. Specifically, naturally occurring polyphenols, such as vescalagin, are water soluble, but are not particularly palatable, being highly tannic in flavor. Ethoxylation under acidic conditions at pH 3.0 to 4.5 results in ethyl vescalagin, which is more lipophilic than vescalagin, and therefore is a fat-soluble polyphenol, and effectively a pro-drug.

In a highly acidic, environment, for example, at pH 1.8, as would be found in the mammalian stomach or an artificial bioreactor, ethyl vescalagin is ‘digested’ to give ellagic acid (a water-soluble polyphenol), which is poorly absorbed and ethyl vescalin (a fat-soluble polyphenol), which can travel via the bloodstream to the target or organ.

A general procedure for the preparation of the modified oak C-glycosidic ellagitannins, where the modification is ethoxylation, preferably entails the following steps:

(i) extracting oak C-glycosidic ellagitannins from oak wood chips using an acidic ethanolic solution; and
(ii) treating the extract to remove any remaining alcohol, thereby obtaining a mixture of ethoxylated oak C-glycosidic ellagitannins.

Extraction of the oak wood chips using an acidic ethanolic solution as in step (i) results in ethoxylation of about 50% up to about 95% of the oak C-glycosidic ellagitannins present in the oak wood chips. Thus, extraction of oak wood chips using an acidic ethanolic solution can result in ethoxylation of about 50%, about 55%, about 60%, about 65%, about 70%, about 75%, about 80%, about 85%, about 90%, or about 95% of the oak C-glycosidic ellagitannins present in the oak wood chips. The oak wood chips can be from any suitable oak species, including European red oak, (Quercus robur, Q. petraea), North American white oak (Q. alba), French oak (Q. robur, Q. petraea) and Japanese oak (Lithocarpus glaber). A preferred oak for use in the invention, is European oak, which contains about five times more C-glycosidic ellagitannins than American oak.

Preferably, the oak wood chips are toasted, or are prepared from toasted oak, however, untoasted oak wood chips or oak wood chips prepared from untoasted oak can also be used.

Preferably, the acidic ethanolic solution used in step (i) is within the pH range of about pH 3.0 to about pH 4.5, and the extraction period is within the range of about 10 days to about 30 days. The acidic ethanolic solution can thus have a pH of 3.0, 3.1, 3.2, 3.3, 3.4, 3.5, 3.6, 3.7, 3.8, 3.9, 4.0, 4.1, 4.2, 4.3, 4.4 or 4.5, and the extraction period can be for 10, 11, 12, 13, 14, 15, 16, 17, 18, 19, 20, 21, 22, 23, 24, 25, 26, 27, 28, 29 or 30 days. Particularly preferred is an extraction period of about 20 days (to achieve optimal ethoxylation).

Preferably the acidic ethanolic solution comprises about 20% to about 100% v/v food-grade alcohol. The acidic ethanolic solution can thus comprise about 20%, about 30%, about 40%, about 50%, about 60%, about 70%, about 80% or about 90% v/v food-grade alcohol.

Treatment of the extract from step (i) to remove alcohol remaining after the extraction, as in step (ii), can be undertaken using any suitable method known in the art. Preferably, the remaining alcohol can be removed by distillation. In a particularly preferred embodiment, the distillation is vacuum distillation.

The general procedure can optionally include the introduction of one or more anthocyanins to the extraction mixture of step (i). The addition of one or more anthocyanins results in ethoxylated C-glycosidic ellagitannins and C-glycosidic ellagitannin-anthocyanin adducts.

Alternatively, the general procedure can optionally include the introduction of one or more curcumins to the extraction mixture of step (i). The addition of one or more curcumins results in ethoxylated C-glycosidic ellagitannins and C-glycosidic ellagitannin-curcumin adducts.

In a further alternative, the general procedure can optionally include the introduction of one or more curcumins and one or more anthocyanins to the extraction mixture of step (i). The addition of one or more curcumins and one or more anthocyanins results in ethoxylated C-glycosidic ellagitannins, C-glycosidic ellagitannin-curcumin adducts and C-glycosidic ellagitannin-anthocyanin adducts.

By way of illustration only, preferred embodiments of the present invention are described in detail, with reference to the following series of examples.

Note that it was necessary to carry out human trials and these trials were all carried out in secrecy/in confidence.

### BRIEF DESCRIPTION OF THE EXAMPLES

Example 1. EVC successfully treats and prevents metabolic syndrome.

Example 2. Low dose EVC superior to high dose ellagic acid in treatment and prevention of metabolic syndrome.

Example 3. EVC/non-ethoxylated ground coffee combination successfully treats and prevents metabolic syndrome.

Example 4. Well tolerated, high dose EVC/ethoxylated black tea successful outcomes in metabolic syndrome, cancer, arthritis, gout, cholesterol, less exercise induced muscular stiffness, enlarged spleen pain, bronchial pneumonia recovery, chronic fatigue syndrome, mental acuity, alertness in evening, more energy, more motivation for exercise, erectile function improvement, weight loss, reduced chemotherapy side effects, alopecia associated with chemotherapy.

Example 5. High dose EVC/ethoxylated ground coffee successful and rapid treatment of arthritis.

Example 6. High dose EVC/ethoxylated green tea as a palatable beverage option.

Example 7. Medium dose EVC/ethoxylated black tea production process results in natural de-caffeination.

Example 8. Medium dose EVC/ethoxylated ground coffee production process results in natural de-caffeination.

Example 9. Ethoxylation process conditions with caffeinated foods results in caffeine reduction and improved anti-inflammatory capability.

Example 10. Medium dose EVC/non-ethoxylated instant coffee is pleasant, well tolerated and provides successful results in osteoarthritis pain relief, medication replacement and a feeling of wellness. Also outlined is the production process for EVC/ethoxylated instant coffee.

Example 11. Medium dose EVC fortified wines are pleasant tasting, well tolerated and result in reduced muscle stiffness associated with exercise.

Example 12. Versatile EVC/grape powder for beverage, food additive and dietary supplement applications.

Example 13. Versatile EVC/purple carrot powder for beverage, food additive and dietary supplement applications.

Example 14. EVC incorporated into actively growing fruit.

Example 15. EVC incorporated into post-harvest fruit.

Example 16. EVC incorporated into beer formulations to produce beer with improved taste profiles, well tolerated and with successful health outcomes namely, medication replacement, gout and arthritis.

Example 17. EVC incorporated into onion and garlic powders and their applications as a food additive and dietary supplement.

Example 18. Versatile EVC/non-ethoxylated cocoa powder for beverage, food additive and dietary supplement applications. Also outlined is the production process for EVC/ethoxylated cocoa.

Example 19. Fractionation of EVC and its application as a flavouring and dietary supplement.

Example 20. Hydrolysed EVC gel production and applications.

Example 21. Suboptimal metabolic syndrome results when oak/catechin compete with ethoxylated oak production and implications when coffee and chocolate combinations are tested.

Example 22. Low dose non-ethoxylated American oak/ethoxylated green tea/ethoxylated olive leaf extract formulation as a palatable beverage option and favourable metabolic syndrome outcomes superior in respect to hypertension and comparable to phospholipase A2 inhibitor and ibuprofen in respect to other metabolic syndrome parameters. Human testimonial on fertility.

Example 23. Low dose EVC/ethoxylated purple carrot formulation delivers favourable health outcomes, namely arthritis and migraine prevention.

Example 24. Low dose EVC/ethoxylated purple carrot formulation prevented the increase in RANKL:OPG ratio that characterizes osteoporosis.

Example 25. Medium dose EVC/non-ethoxylated purple carrot wine is pleasant tasting, well tolerated and produces favourable health outcomes, namely with respect to chronic fatigue syndrome and arthritis.

Example 26. Medium dose EVC/ethoxylated turmeric formulation is pleasant tasting, well tolerated and produces favourable health outcomes, namely with respect to cancer, libido improvement, cold/flu avoidance, eczema, alopecia, hair re-growth, immune enhancement, reduction of “flu like symptoms” with chemotherapy and rapid recovery from penicillin resistant infections.

Example 27. Low dose EVC/non-ethoxylated purple carrot and elderberry combination with favourable arthritis results.

Example 28. Low dose EVC/non-ethoxylated purple carrot and camu camu combination with favourable arthritis results.

Example 29. Low dose EVC/non-ethoxylated purple carrot/fucoidan formulation with excellent arthritis results.

Example 30. High dose EVC/non-ethoxylated purple carrot/fucoidan formulation in lipopolysaccharide-mediated acute lung inflammation.

Example 31. Suboptimal metabolic syndrome results (notably abdominal fat reduction) when oak/green tea catechin hybrids compete with ethoxylated oak production in this formulation that also incorporates omega-3 oil.

Example 32. Suboptimal metabolic syndrome results when oak/green tea catechin hybrids compete with ethoxylated oak production in this instant tea formulation.

Example 33. Suboptimal cancer results when oak/green tea catechin hybrids compete with ethoxylated oak production in this instant tea formulation.

Example 34. Medium dose EVC/ethoxylated turmeric formulation is pleasant tasting, well tolerated and produces favourable health outcomes, namely for cancer, pain associated with bone cancer, cold sores, ulcers, moods and dementia.

Example 35. Medium dose EVC/non-ethoxylated food extract formulation produces significant results in cancer re-occurrence in-vitro/human and favourable health outcomes in cancer, life extension, dementia, aggression associated with dementia, cold sores, ulcers and pain associated with bone cancer.

Example 36. Low dose EVC/ethoxylated food extract is unpleasant tastewise, but is well tolerated and produces significant health outcomes in cancer.

Example 37. Low dose EVC/ethoxylated food extract/non-ethoxylated green tea formulation produces significant health outcomes in cancer.

Example 38. Low dose EVC/ethoxylated food extract (Example 35) cancer results are improved by the addition of anthocyanin rich fruit juice concentrates to the formulation.

Example 39. Low dose EVC/non-ethoxylated food extracts/ethoxylated food extracts/anthocyanin rich non-ethoxylated fruit juice concentrates with significant cancer results.

### Examples

### Example 1

### EVC Successfully Treats and Prevents Metabolic Syndrome

This example describes the preparation of ethoxylated vescalagin/castalagin rich extract (EVC) and use of the extract in the treatment and prevention of metabolic syndrome.

Materials and Methods

Ethoxylated Vescalagin/Castalagin Rich Extract (EVC)

European oak wood (Querces petraea Liebl) was converted into chips (1 kg) and extracted for 6 months at room temperature in grape brandy (4 kg, pH 3:5, containing 62% v/v alcohol). The resultant alcoholic extract was filtered to remove the solid Material and de-alcoholised in a vacuum distiller to 25% of its original volume. The resultant extract is an ethoxylated vescalagin/castalagin rich extract, named EVC. The resultant de-alcoholised extract (1 kg) was derived from 1 kg oak chips, and is thus designated EVC 1:1.

The EVC was analysed by liquid chromatography/mass spectrometry (LC/MS) employing electrospray mass spectrometry and ultraviolet detection. The analysis was performed on a Micromass Quattro micro tandem quadrupole mass spectrometer (Waters, Manchester, UK). LC separation was provided by a Waters liquid chromatograph (Waters, Milford, USA), consisting of a 2,695 separation module and 2,487 dual-wavelength ultraviolet detector. Data were acquired by the Masslynx data system for both the MS and ultraviolet data. For LC, a flow rate of 1 mL/min was used with 0.1% aqueous formic acid and methanol as solvent and injection volume of 20 μL. For UV detection, 254 and 280 nm wavelengths were used.

Pure castalagin, vescalagin, grandinin and roburin E were used as the monomer standards in this procedure. There were 20% uncertainties in the measurements of ellagitannins derived from the contributions of the uncertainties in the preparation and analysis of the standards and samples.

The chemical profile of the EVC with respect to monomeric oak ellagitannins is set out in Table 1.

TABLE 1

| monomeric oak ellagitannin | amount in EVC (mg/mL) |
|---|---|
| Vescalagin | 1.2 |
| Castalagin | 0.8 |
| Roburin E | 0.8 |
| Grandinin | 2 |
| Ellagic acid | 0.1 |

The total monomeric oak ellagitannins in the EVC is thus 4.9 mg/mL. The EVC was tested in high-carbohydrate, high-fat diet-induced metabolic syndrome in rats as detailed below.

Rats and Diets

Male Wistar rats (8 weeks old, weighing 328±2 g, n40) and male spontaneously hypertensive rats (SHR) (42 weeks old, weighing 422±6 g, n=20) were used.

Male Wistar rats were randomly divided into four experimental groups and were fed with either cornstarch diet (C; n=10), cornstarch diet+EVC (0.5 mL/kg food; CE; n=10), high-carbohydrate, high-fat diet (H; n=10) or high-carbohydrate, high-fat diet+EVC (0.5 mL/kg food; HE; n=10) for 16 weeks. CE and HE rats were fed with respective diets for the first 8 weeks without EVC; EVC was supplemented in the diets of CE and HE rats for the last 8 weeks of the protocol. Compositions of H and C diets used in this study are as previously described in Panchal S K, Ward L and Brown L, ‘Ellagic acid attenuates high-carbohydrate, high-fat diet-induced metabolic syndrome in rats’, Eur. J. Nutr., DOI: 10.1007/s00394-012-0358-9 (2012).

Male adult SHR with high and stable systolic blood pressure were divided into two groups of 10 rats each, one without treatment (S) and one with EVC treatment in diet (SE; 0.5 mL/kg in food) for 12 weeks. All SHR were fed on standard powdered chow diet (Specialty Feeds, Glen Forest, WA, Australia). All rats were given ad libitum access to food and water and were individually housed in temperature-controlled 12-hour light-dark conditions. Energy intakes were calculated as previously described in Pancahl S K et al. (2012).

Physiological Measurements

Body weight, food and water intakes were measured daily for all rats. Abdominal circumference and body length were measured at the end of the protocol using a standard measuring tape under light anaesthesia with Zoletil (tiletamine 10 mg/kg, zolazepam 10 mg/kg, i.p.). Energy intake, body mass index and feed efficiency were calculated as previously described in Pancahl S K et al. (2012).

Systolic Blood Pressure Measurements

Systolic blood pressure of rats was measured at the end of the protocol under light anaesthesia with Zoletil (tiletamine 10 mg/kg, zolazepam 10 mg/kg, i.p.), using an MLT1010 Piezo-Electric Pulse Transducer and inflatable tail cuff connected to a MLT844 Piezo-Electric Pressure Transducer and PowerLab data acquisition unit as previously described in Pancahl S K et al. (2012).

Echocardiography

Echocardiographic examinations (Phillips iE33, 12-MHz transducer) were performed in all rats at the end of protocol as previously described (Panchal S K et al. (2012)). Briefly, rats were anaesthetized using Zoletil (tiletamine 25 mg/kg and zolazepam 25 mg/kg, i.p.) and Ilium Xylazil (xylazine 15 mg/kg, i.p.) and positioned in dorsal recumbency. Electrodes attached to the skin overlying the elbows and right stifle facilitated the simultaneous recording of a lead II electrocardiogram (Panchal S K et al. (2012)).

Body Composition Measurements

Dual-energy X-ray absorptiometric measurements were performed at the end of the protocol using a Norland XR36 DXA instrument under anaesthesia with Zoletil (tiletamine 25 mg/kg and, zolazepam 25 mg/kg, i.p.) and Ilium Xylazil (xylazine 15 mg/kg, i.p.). Scans were analysed using the manufacturer's recommended software for use in laboratory animals (Small Subject Analysis Software, version 2.5.3/1.3.1). The precision error of lean mass for replicate measurements, with repositioning, was 3.2%.

Oral Glucose Tolerance Test

At the end of the protocol, rats were deprived of food for 12 h for oral glucose tolerance testing. During this food deprivation period, fructose-supplemented drinking water in H and HE groups was replaced with normal drinking water. Oral glucose tolerance tests were performed after determining basal blood glucose concentrations in tail vein blood using Medisense Precision Q.I.D. glucose meters. Rats were given a glucose load of 2 g/kg body weight as 40% glucose solution via oral gavage and blood glucose concentrations were measured again 30, 60, 90 and 120 min after oral glucose administration (Panchal S K et al. (2012)). Blood glucose concentrations over the period of 120 min were used to calculate area under the curve.

Terminal Experiments

Rats were euthanised with Lethabarb (pentobarbitone sodium, 100 mg/kg, i.p.). After euthanasia, heparin (200 IU) was injected through the right femoral vein. The abdomen was then opened and blood (5 mL) was withdrawn from the abdominal aorta and collected into heparinised tubes. Blood was centrifuged at 5,000 g for 15 min to obtain plasma. Hearts were removed and were used as an isolated Langendorff heart preparation.

Isolated Langendorff Heart Preparation

The isolated Langendorff heart preparation assessed left ventricular function of the rats in all the groups as in previous studies (Panchal S K et al. (2012)). Hearts isolated from euthanised rats were perfused with modified Krebs-Henseleit bicarbonate buffer bubbled with 95% O₂-5% CO₂ and maintained at 35° C. Isovolumetric ventricular function was measured by inserting a latex balloon catheter into the left ventricle connected to a Capto SP844 MLT844 physiological pressure transducer and Chart software on a Maclab system. All left ventricular end-diastolic pressure values were measured during pacing of the heart at 250 beats/min using an electrical stimulator. End-diastolic pressures were obtained from 0 to 30 mmHg for the calculation of diastolic stiffness constant (j, dimensionless) as described in previous studies (Panchal S K et al. (2012)).

Vascular Reactivity

Thoracic aortic rings (4 mm in length; n=11-12 from each group) were suspended in an organ bath filled with Tyrode physiological salt solution bubbled with 95% O₂-5% CO₂, maintained at 35° C. and allowed to stabilise at a resting tension of 10 mN. Cumulative concentration-response curves (contraction) were obtained for noradrenaline and cumulative concentration-response curves (relaxation) were obtained for sodium nitroprusside and acetylcholine following submaximal (70%) contraction to noradrenaline (Panchal S K et al. (2012)).

Organ Weights

After isolated heart perfusion studies, hearts (n=8-9 from each group) were separated into left ventricles (with septum) and right ventricles and weighed. Livers (n=8-9 from each group) were isolated and weighed. Retroperitoneal, epididymal and omental abdominal fat pads were removed separately and weighed. These organ weights were normalised against the tibial length (48.2±0.1 mm, n=35) at the time of organ removal and expressed as mg/mm of tibial length (Panchal S K et al. (2012)).

Histology

Histology of the Heart

Hearts were removed from the rats (n=3 from each group) soon after euthanasia and these hearts were fixed in 10% neutral buffered formalin for 3 days. The samples were then dehydrated and embedded in paraffin wax. Thin sections (5 μm) of left ventricle were cut and stained with haematoxylin and eosin to study infiltration of inflammatory cells and picrosirius red to study collagen deposition (Panchal S K et al. (2012)).

Histology of the Liver

Liver portions were isolated (n=3) and fixed in 10% neutral buffered formalin for three days. These tissue samples were dehydrated and then embedded in paraffin wax. Thin sections (5 μm) of these tissues were cut and stained with haematoxylin and eosin for the determination of inflammatory cell infiltration (20×) and for determining the fat vacuoles (40×) in liver. Liver sections were also stained with Milligan's trichrome stain to determine portal fibrosis (20×) (Panchal S K et al. (2012)).

Plasma Biochemistry

Plasma concentrations of total cholesterol and triglycerides were determined using kits and controls supplied by Olympus using an Olympus AU 400 analyser (Panchal S K et al. (2012)).

Non-esterified fatty acids (NEFA) in plasma were determined using a commercial kit (Wako, Osaka, Japan). Plasma activity of alanine transaminase (ALT), aspartate transaminase (AST), alkaline phosphatase (ALP), lactate dehydrogenase (LDH) and the concentrations of albumin, total bilirubin, urea and uric acid were determined using kits and controls supplied by Olympus using an Olympus analyser (AU 400, Tokyo, Japan) (Panchal S K et al. (2012)). Plasma C-reactive protein (BD Bioscience, Brisbane, Australia) concentrations were measured using commercial kits according to manufacturer-provided standards and protocols.

Regulatory Protein Levels in the Heart and the Liver

Heart and liver samples isolated from rats (n=3 from each group) were stored at −80° C. These samples were homogenised and sonicated after adding cell lysis buffer followed by ultracentrifugation at 100,000 g for 30 min at 4° C. Supernatants were used to measure protein concentration in each sample by bicinconinic acid method (Thermo Scientific). Supernatants with equal protein amounts (40 μg) from each group were used in Western blot analysis to study the protein levels of CPT-1, Nrf2 (antibodies from Santa Cruz Biotechnology, Santa Cruz, Calif.), NF-jB (antibody from Cell Signaling Technology, Danvers, Mass.) and β-actin (antibody from Sigma-Aldrich Corp., St. Louis, Mo.) in the heart and the liver.

Statistical Analysis

Values are presented as mean±SEM. Results were tested for variance using Bartlett's test and variables that were not normally distributed were transformed (using log 10 function) prior to statistical analyses. All the groups were tested for effects of diet (D), treatment (E) and their interactions (D×E) by two-way ANOVA. When interaction and/or the main effects were significant, means were compared using Newman-Keuls multiple comparison post-test. Mean EVC intakes between CE and HE groups were compared using Student's t-test. P<0.05 was considered significant. All statistical analyses were performed using GraphPad Prism version 5.00 for Windows.

Results

Table 2 details the daily intake of ellagitannins in EVC-treated rats.

TABLE 2

| Components of EVC | CE | HE | SE |
|---|---|---|---|
| EVC (μL) | 16.41 ± 0.37 | 10.66 ± 0.48 | 15.21 ± 0.23 |
| Vescalagin (μg) | 19.70 ± 0.44 | 12.79 ± 0.57 | 18.25 ± 0.28 |
| Castalagin (μg) | 13.13 ± 0.30 | 8.52 ± 0.38 | 12.17 ± 0.19 |
| Grandinin (μg) | 32.83 ± 0.74 | 21.31 ± 0.95 | 30.42 ± 0.46 |
| Roburin E (μg) | 13.13 ± 0.30 | 8.52 ± 0.38 | 12.17 ± 0.19 |
| Total ellagitannin intake (μg) | 78.79 ± 1.77 | 51.15 ± 2.29 | 73.00 ± 1.11 |
| Ellagic acid (μg) | 1.64 ± 0.04 | 1.07 ± 0.05 | 1.52 ± 0.02 |

*CE cornstarch diet + EVC-fed rats; HE high-carbohydrate, high-fat diet + EVC-fed rats; SE SHR supplemented with EVC

Values are mean±SEM and n=10 for each group. Daily intake of EVC and individual monomer ellagitannins from tested monomer EVC extract was calculated based on the daily intake of food.

Table 3 details the effects of EVC on H-induced (high carbohydrate, high fat) physiological, metabolic and oxidated stress variables in C, CE, H and HE rats.

TABLE 3

| Variables | C | CE | H | HE |
|---|---|---|---|---|
| Physiological variables | | | | |
| Initial body weight (g) | 334 ± 7 | 329 ± 8 | 332 ± 9 | 336 ± 6 |
| Final body weight (g) | 424 ± 11^{bc} | 401 ± 6^{c} | 510 ± 10^{a} | 440 ± 8^{b} |
| Water intake (mL/day) | 30.2 ± 0.6^{a} | 31.5 ± 0.4^{a} | 20.1 ± 0.4^{b} | 18.6 ± 0.5^{c} |
| Food intake (g/day) | 30.7 ± 0.7^{a} | 32.8 ± 0.8^{a} | 22.5 ± 0.8^{b} | 22.3 ± 1.0^{b} |
| Energy intake (kJ/day) | 345 ± 10^{b} | 365 ± 11^{b} | 479 ± 15^{a} | 476 ± 16^{a} |
| BMI (g/cm²) | 0.70 ± 0.01^{b} | 0.65 ± 0.01^{c} | 0.78 ± 0.01^{a} | 0.69 ± 0.01^{b} |
| Feed efficiency (g/kJ) | 0.26 ± 0.02^{b} | 0.20 ± 0.01^{c} | 0.37 ± 0.02^{a} | 0.22 ± 0.01^{bc} |
| Abdominal circumference | 21.4 ± 0.3^{b} | 19.6 ± 0.2^{c} | 23.8 ± 0.3^{a} | 20.0 ± 0.2^{c} |
| (cm) | | | | |
| Abdominal fat pads (mg/mm | 407 ± 48^{b} | 206 ± 31^{c} | 805 ± 53^{a} | 415 ± 47^{b} |
| tibial length) | | | | |
| Metabolic variables | | | | |
| Basal blood glucose | 4.2 ± 0.2^{b} | 3.8 ± 0.2^{bc} | 4.9 ± 0.2^{a} | 3.3 ± 0.2^{c} |
| (mmol/L) | | | | |
| AUC (mmol/L min) | 688 ± 13^{b} | 626 ± 18^{c} | 768 ± 12^{a} | 664 ± 16^{bc} |
| Plasma total cholesterol | 1.5 ± 0.1^{b} | 1.3 ± 0.1^{b} | 2.0 ± 0.1^{a} | 1.4 ± 0.1^{b} |
| (mmol/L) | | | | |
| Plasma triglycerides | 0.4 ± 0.1^{b} | 0.4 ± 0.1^{b} | 0.9 ± 0.1^{a} | 0.5 ± 0.1^{b} |
| (mmol/L) | | | | |
| Plasma non-esterified fatty | 1.3 ± 0.1^{b} | 1.1 ± 0.1^{b} | 2.6 ± 0.2^{a} | 1.4 ± 0.1^{b} |
| acids (mmol/L) | | | | |
| Plasma urea (mmol/L) | 5.5 ± 0.3^{ab} | 6.1 ± 0.3^{a} | 3.4 ± 0.2^{c} | 4.8 ± 0.3^{b} |
| Plasma uric acid (μmol/L) | 35.2 ± 1.8^{b} | 37.2 ± 2.1^{b} | 52.6 ± 2.6^{a} | 33.1 ± 1.9^{b} |
| Oxidative stress and | | | | |
| inflammatory markers | | | | |
| Plasma malondialdehyde | 27.1 ± 1.1^{b} | 27.5 ± 1.0^{b} | 31.5 ± 1.2^{a} | 28.0 ± 1.2^{b} |
| (μmol/L) | | | | |
| Plasma glutathione | 1,165 ± 39^{a} | 1,135 ± 26^{a} | 880 ± 27^{b} | 1,063 ± 27^{a} |
| peroxidase activity (U/L) | | | | |
| Plasma C-reactive protein | 2.60 ± 0.13^{bc} | 3.27 ± 0.17^{a} | 2.76 ± 0.08^{b} | 2.36 ± 0.10^{c} |
| (μmol/L) | | | | |

*C cornstarch diet-fed rats, CE cornstarch diet + EVC-fed rats; H high-carbohydrate, high-fat diet-fed rats; HE high-carbohydrate, high-fat diet + EVC-fed rats. Values are mean ± SEM and n = 8-10 for each group. Means without a common letter differ, P < 0.05.

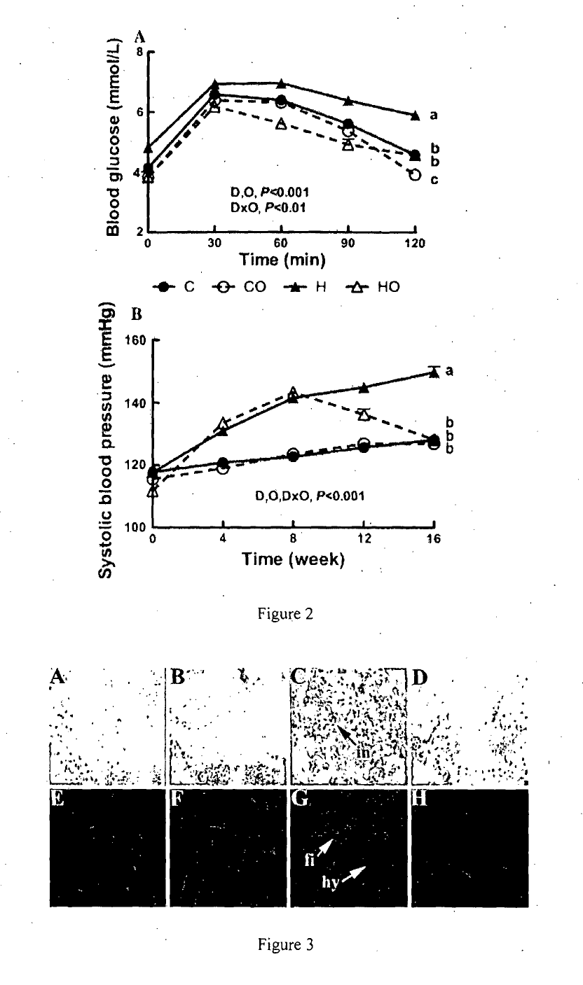

The effects of EVC on oral glucose tolerance (A) and systolic blood pressure (B) are shown in FIG. 2. The values indicated are mean±SEM and n=10 for each group. End-point means without a common letter differ, P<0.05.

D, E and D×E represent effects of diet, EVC and interaction of diet and EVC. C represents cornstarch diet-fed rats, CE re-presents cornstarch diet+EVC-fed rats, H represents high-carbohydrate, high-fat diet-fed rats, and HE represents high-carbohydrate, high-fat diet+EVC-fed rats.

The effects of EVC on cardiovascular and hepatic variables in H-induced metabolic syndrome in C, CE, H and HE rats are detailed in Table 4.

TABLE 4

| Variables | C | CE | H | HE |
|---|---|---|---|---|
| Cardiovascular variables | | | | |
| LVIDd (mm) | 6.46 ± 0.16^{b} | 6.40 ± 0.15^{b} | 7.21 ± 0.20^{a} | 6.56 ± 0.18^{a} |
| LVPWd (mm) | 1.58 ± 0.03^{b} | 1.60 ± 0.04^{b} | 1.75 ± 0.05^{a} | 1.66 ± 0.05^{ab} |
| Systolic volume (μL) | 54.6 ± 5.1^{b} | 50.2 ± 4.0^{b} | 115.1 ± 4.8^{a} | 58.9 ± 4.1^{b} |
| Relative wall thickness | 0.49 ± 0.01 | 0.48 ± 0.01 | 0.49 ± 0.01 | 0.49 ± 0.01 |
| Fractional shortening (%) | 53.2 ± 1.3^{a} | 52.2 ± 1.4^{a} | 42.4 ± 1.0^{b} | 51.0 ± 1.5^{a} |
| Ejection fraction (%) | 85.1 ± 1.2^{a} | 82.6 ± 1.4^{a} | 73.0 ± 1.1^{b} | 82.1 ± 1.4^{a} |
| Estimated left ventricular | 0.69 ± 0.02^{b} | 0.68 ± 0.02^{b} | 0.82 ± 0.03^{a} | 0.71 ± 0.02^{b} |
| mass (g) | | | | |
| Left ventricular + septum wet | 20.1 ± 0.8^{b} | 19.4 ± 0.7^{b} | 22.5 ± 0.8^{a} | 19.9 ± 0.6^{b} |
| weight (mg/mm tibial length) | | | | |
| Right ventricular weight | 4.1 ± 0.3 | 4.3 ± 0.3 | 4.5 ± 0.4 | 4.2 ± 0.3 |
| (mg/mm tibial length) | | | | |
| Left ventricular diastolic | 19.1 ± 0.8^{c} | 18.4 ± 0.9^{c} | 28.2 ± 1.1^{a} | 22.8 ± 10.8^{b} |
| stiffness constant, κ | | | | |
| Hepatic variables | | | | |
| Liver wet weight (mg/mm | 265 ± 10^{b} | 232 ± 11^{b} | 297 ± 9^{a} | 256 ± 10⁶ |
| tibial length) | | | | |
| Plasma ALT (U/L) | 35.6 ± 0.7^{b} | 32.5 ± 0.6^{c} | 48.2 ± 0.6^{a} | 36.9 ± 0.9^{b} |
| Plasma AST (U/L) | 75.1 ± 4.8^{b} | 77.8 ± 5.3^{b} | 101.2 ± 6.6^{a} | 82.3 ± 5.9^{b} |
| Plasma ALP (U/L) | 165 ± 15^{b} | 172 ± 15^{b} | 257 ± 20^{a} | 192 ± 18^{b} |
| Plasma LDH (U/L) | 220 ± 20^{b} | 206 ± 22^{b} | 451 ± 28^{a} | 262 ± 25^{b} |
| Plasma albumin (g/L) | 27.9 ± 0.3 | 28.1 ± 0.3 | 28.5 ± 0.3 | 28.0 ± 0.3 |
| Plasma total bilirubin | 2.0 ± 0.1^{b} | 1.9 ± 0.1^{b} | 2.5 ± 0.1^{a} | 2.0 ± 0.1^{b} |
| (μmol/L) | | | | |

*C cornstarch diet-fed rats; CE cornstarch diet + EVC-fed rats; HE high-carbohydrate, high-fat, diet-fed rats; HE high-carbohydrate, high-fat diet + EVC-fed rats. Values are mean ± SEM and n = 8-10 for each group. Means without a common letter differ, P < 0.05.

The effects of EVC on inflammation and fibrosis in the heart induced by H feeding are illustrated in FIG. 3. Haematoxylin and eosin staining of left ventricle showing infiltration of inflammatory cells, where the inflammatory cells are seen as dark spots in A—cornstarch diet-fed rats, B—cornstarch diet+EVC-fed rats, C—high-carbohydrate, high-fat diet-fed rats, and D—high-carbohydrate, high-fat diet+EVC-fed rats.

Picrosirius red staining of the left ventricle showing collagen deposition is marked as fi (fibrosis) and by (hypertrophied cardiomyocytes) in E—cornstarch diet-fed rats, F—cornstarch diet+EVC-fed rats, G—high-carbohydrate, high-fat diet-fed rats, and H—high-carbohydrate, high-fat diet+EVC-fed rats.

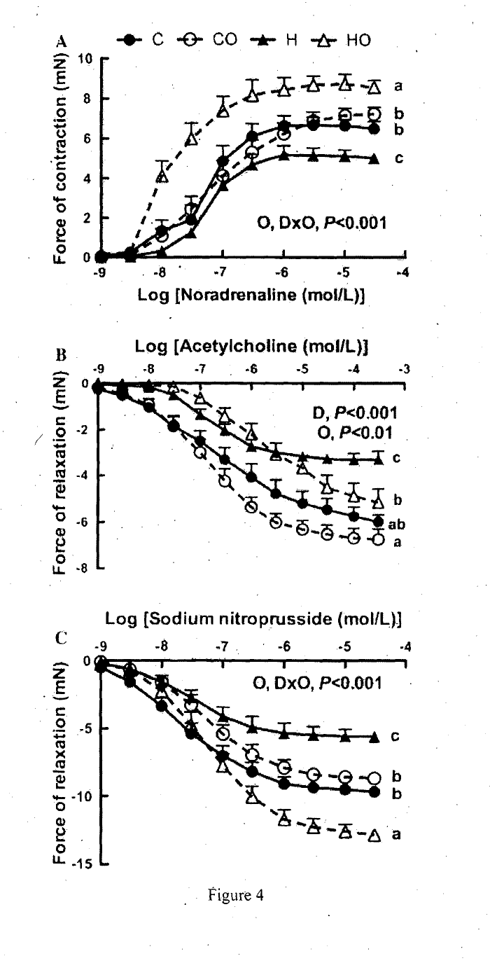

The effects of EVC on vascular responses in rats are shown in FIG. 4. The vascular responses in thoracic aortic rings from C, CE, H and HE rats monitored are (A) noradrenaline-induced contraction, (B) acetylcholine-induced relaxation and (C) sodium nitroprusside-induced relaxation. The values indicated are mean±SEM and n=10 for each group. End-point means without a common letter differ, P<0.05.

D, E and D×E represent effects of diet, EVC and interaction of diet and EVC, respectively. C represents cornstarch diet-fed rats, CE represents cornstarch diet+EVC-fed rats, H represents high-carbohydrate, high-fat diet-fed rats, and HE represents high-carbohydrate, high-fat diet+EVC-fed rats.

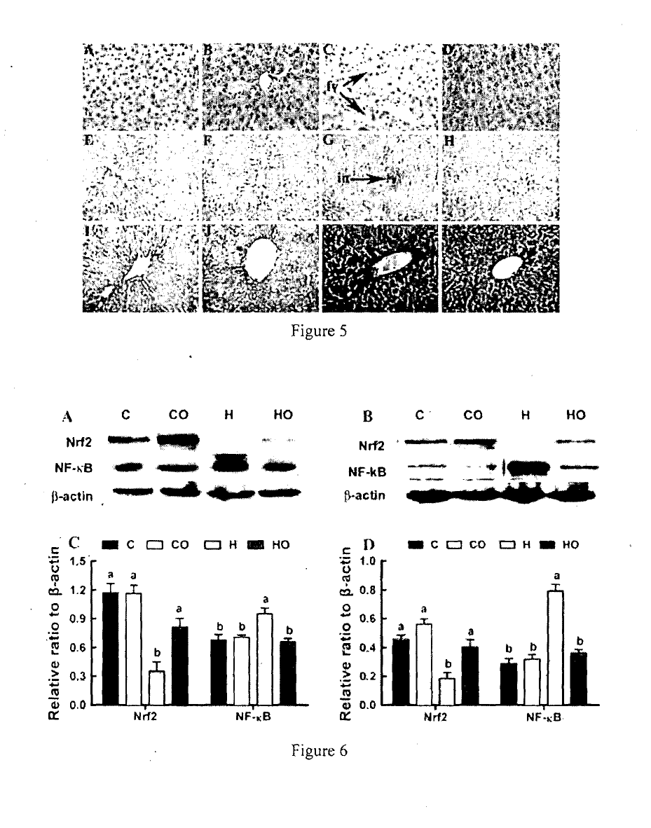

The effects of EVC on fat deposition, inflammation and fibrosis in rat livers are shown in FIG. 5. Haematoxylin and eosin staining of liver showing enlarged fat vacuoles marked as ‘fv’ in A—cornstarch diet-fed rats, B—cornstarch diet+EVC-fed rats, C—high-carbohydrate, high-fat diet-fed rats, and D—high-carbohydrate, high-fat diet+EVC-fed rats. Inflammatory cells marked as ‘in’ in E—cornstarch diet-fed rats, F—cornstarch diet+EVC-fed rats, G—high-carbohydrate, high-fat diet-fed rats, and H—high-carbohydrate, high-fat diet+EVC-fed rats. Milligan's Trichrome staining of hepatic portal regions showing fibrosis marked as in I—cornstarch diet-fed rats, J—cornstarch diet+EVC-fed rats, K—high-carbohydrate, high-fat diet-fed rats, and L—high-carbohydrate, high-fat diet+EVC-fed rats.

The effects of EVC on expression of Nrf2 and NF-κB in the heart (A) and liver (B) are shown in FIG. 6. For quantitative analysis, the expression of these proteins was normalized against the expression of β-actin in the heart (C) and the liver (D). Values are mean±SEM and n=3. Means without a common letter differ, P<0.05. C represents cornstarch diet-fed rats, CE represents cornstarch diet+EVC-fed rats, H represents high-carbohydrate, high-fat diet-fed rats, and HE represents high-carbohydrate, high-fat diet+EVC-fed rats.

The effects of EVC on cardiovascular structure and function in SHR are detailed in Table 5.

TABLE 5

| Variables | S | SE |
|---|---|---|
| LVIDd (mm) | 8.01 ± 0.28 | 7.05 ± 0.22* |
| LVPWd (mm) | 1.86 ± 0.08 | 1.72 ± 0.06 |
| Systolic volume (μL) | 138 ± 4 | 112 ± 4*** |
| Relative wall thickness | 0.54 ± 0.01 | 0.53 ± 0.01 |
| Fractional shortening (%) | 32.2 ± 1.2 | 38.5 ± 1.4** |
| Ejection fraction (%) | 68.1 ± 1.0 | 74.6 ± 1.2*** |
| Estimated left ventricular mass (g) | 0.96 ± 0.04 | 0.82 ± 0.04* |
| Left ventricular + septum wet weight | 28.2 ± 0.8 | 25.3 ± 0.6** |
| (mg/mm tibial length) | | |
| Right ventricular weight (mg/mm tibial | 4.5 ± 0.3 | 4.5 ± 0.2 |
| length) | | |
| Left ventricular diastolic stiffness | 32.4 ± 1.3 | 28.6 ± 1.1* |
| constant, κ | | |

Values are mean ± SEM and n = 10 for each group.
*versus S differ P < 0.05,
**versus S differ P < 0.01, and
***versus S differ P < 0.001

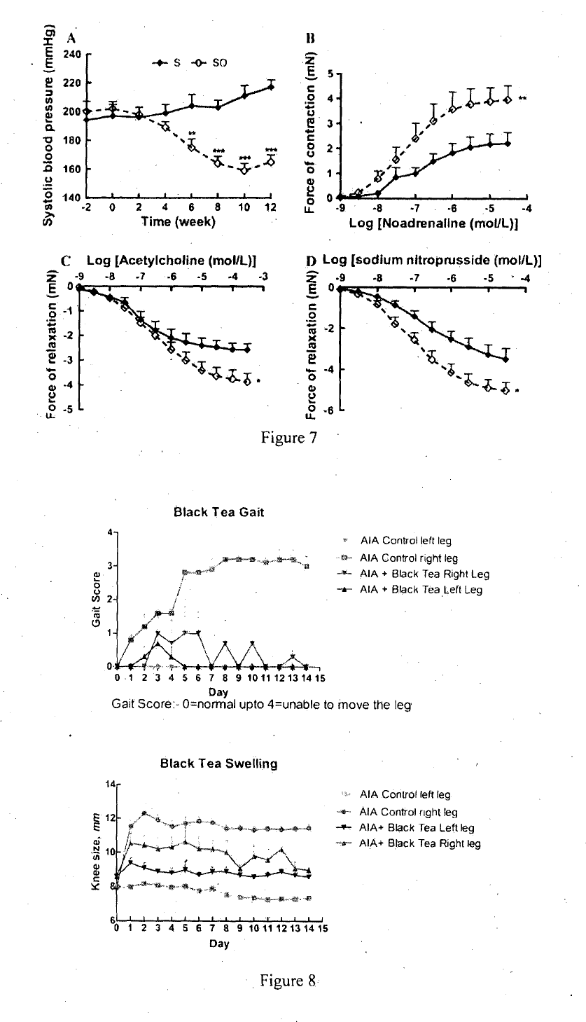

The effects of EVC on (A) systolic blood pressure, (B) vascular contraction by noradrenaline, (C) vascular relaxation by acetylcholine, and (D) vascular relaxation by sodium nitroprusside in SHR (S) and SHR supplement with EVC (SE) are shown in FIG. 7.

Discussion

The results show that an ellagitannin mixture extracted from European oak produces both cardiac and liver protection as well as improved metabolic profile in high-carbohydrate, high-fat diet-fed Wistar rats. In addition, EVC improved cardiovascular structure and function in SHR.

H feeding in rats leads to the development of signs of metabolic syndrome and associated end-organ damage. H rats developed abdominal obesity, hypertension, dyslipidaemia and impaired glucose tolerance. These changes were accompanied by cardiovascular remodelling and non-alcoholic steatohepatitis (Panchal S K et al. (2012)). Thus, rats fed with H diet are suitable as a model to demonstrate the major changes found in human metabolic syndrome (Panchal S K et al. (2012)).

The reduced plasma malondialdehyde concentrations and increased plasma glutathione peroxidase activity along with increased expression of Nrf2 in both the heart and the liver strongly support an antioxidant mechanism. Without wishing to be bound by theory, the inventors believe that the antioxidant activity of ellagitannins may lead to higher nitric oxide bioavailability by removal of superoxide, leading to reduction in blood pressure.

Increased expression of endothelial nitric oxide synthase (eNOS), as shown with punicalagin in hypercholesterolaemic mice, would also increase nitric oxide bioavailability. Further, the improvement in vascular relaxation responses of EVC-treated rats is supportive of a reduction in, blood pressure, especially the improvement in acetylcholine-induced relaxation, by a response dependent on endothelium-derived nitric oxide.

The results indicated that the anti-inflammatory mechanism is supported by the lower expression of NF-κB, lower infiltration of inflammatory cells in the heart and lower collagen deposition in EVC-treated rats. These outcomes are associated with lower ventricular stiffness, possibly improving ventricular function.

The hepatoprotective responses with oak-derived ellagitannins are shown by reduced plasma activities of transaminases, attenuation of fat deposition and fibrosis, and inhibition of infiltration of inflammatory cells in the liver. The altered expression of NF-κB and Nrf2 in the liver confirms the antioxidative and anti-inflammatory effects of ellagitannins from EVC. Thus, ellagitannins derived from oak wood ameliorated the changes associated with diet-induced cardiovascular remodelling and non-alcoholic fatty liver disease probably by both antioxidant and anti-inflammatory mechanisms.

EVC-treated rats presented improved metabolic parameters including lower abdominal fat deposition and improved glucose tolerance, as well as protection of the heart and the liver.

It is possible that decreased oxidative stress and inflammation may be at least partially responsible for improving metabolic parameters.

The improved cardiovascular parameters were also measured in adult SHR, the genetic model of choice to mimic human essential hypertension with extensive cardiovascular remodelling. EVC-treated rats had lower systolic blood pressure, ventricular collagen deposition and diastolic cardiac stiffness.

Improvement in vascular responses in EVC-treated SHR also suggests higher bioavailability of nitric oxide. Hence, the results with SHR support the cardioprotective roles of ellagitannins derived from oak wood.

### Example 2

### Low Dose EVC Superior to High Dose Ellagic Acid in Treatment and Prevention of Metabolic Syndrome

A study by Panchal et al., (Panchal S K et al. (2012)), investigating the effect of ellagic acid on high-carbohydrate, high-fat diet-induced metabolic syndrome in rats found that ellagic acid improved hepatic and cardiovascular structure and function, and normalized metabolic parameters such as glucose tolerance, blood lipid components, central obesity and physiological parameters such as body weight.

In addition, there was a reduction in abdominal fat deposition without any change in whole-body fat, indicative of the lipid redistribution seen following consumption of α-linolenic acid-rich chia seeds. In the rats with metabolic syndrome, the redistribution of fat was accompanied by a reduction in blood lipid components, hepatic steatosis and increased fatty acid oxidation. There was also observed an attenuation of oxidative stress and inflammation in the heart and liver.

The authors thus concluded that high-carbohydrate, high-fat diet-induced symptoms of metabolic syndrome in rats were reversed by ellagic acid, accompanied by changes in protein levels of Nrf2, CPTI and NF-κB. The results indicate that these proteins play important roles in the damage associated with metabolic syndrome and that targeting these proteins with natural products can attenuate the complications in metabolic syndrome.

Table 6 provides a ready comparison of the results in a mammalian model of metabolic syndrome of EVC and ellagic acid.

TABLE 6
Effects of EVC (HEVC) and ellagic acid (HEA) supplementation on
physiological and metabolic variables in high-carbohydrate, high fat
diet-fed rats

| Variables | HEVC | HEA |
|---|---|---|
| Physiological variables | | |
| Initial body weight (g) | 336 ± 6 | 336 ± 2 |
| Final body weight (g) | 440 ± 8 | 473 ± 2 |
| Water intake (mL) | 18.6 ± 5 | 19.0 ± 0.4 |
| Food intake (g) | 22.3 ± 1 | 21.0 ± 0.4 |
| BMI (g/cm²) | 0.69 ± 0.01 | 0.72 ± 0.02 |
| Feed efficiency (g/kJ) | 0.22 ± 0.01 | 0.31 ± 0.02 |
| Abdominal circumference (cm) | 20.0 ± 0.2 | 21.3 ± 0.3 |
| Abdominal fat pads (mg/mm tibial | 415 ± 47 | 520 ± 29 |
| length) | | |
| Metabolic variables | | |
| Basal blood glucose (mmol/L) | 3.3 ± 0.2 | 3.9 ± 0.2 |
| AUC (mmol/L min) | 664 ± 16 | 696 ± 11 |
| Plasma total cholesterol (mmol/L) | 1.4 ± 0.1 | 1.5 ± 0.1 |
| Plasma triglycerides (mmol/L) | 0.5 ± 0.1 | 0.5 ± 0.1 |
| Plasma non-esterified fatty acids | 1.4 ± 0.1 | 1.7 ± 0.2 |
| (mmol/L) | | |
| Plasma urea (mmol/L) | 4.8 ± 0.3 | 6.2 ± 0.4 |
| Plasma uric acid (μmol/L) | 33.1 ± 1.9 | 44.3 ± 3.8 |
| Plasma C-reactive protein (μmol/L) | 2.36 ± 0.10 | 2.40 ± 0.07 |

From this summary of the results, in can be seen that EVC outperformed ellagic acid in final body weight, abdominal pad pads, basal blood glucose, AIC (area under the curve), plasma non-esterified fatty acids, plasma urea and plasma uric acid. In the other parameters EVC performed similarly to ellagic acid.

It is important to take into account the doses of EVC and ellagic acid when comparing the results of these two products in the same model of metabolic syndrome. Specifically, the dose of ellagic acid used in Panchal et al. (2012), was 0.8 g/kg food, which equates to a total daily intake of 35.6 mg/kg of pure ellagic acid. In contrast, in the present case, the feeding dose of EVC was 0.5 mL/kg food with the EVC comprising total monomeric oak ellagitannins of 4.9 mg/mL. The benefit of EVC in the mammalian model of metabolic syndrome is thus clearly evident, particularly when it is considered that the EVC-supplement diet contained 0.14% (0.05115 mg/35.6 mg) equivalent of measured total ellagitannin content compared to the ellagic acid-supplemented diet (almost 700 times less) but achieved greater or similar health outcomes.

### Example 3

### EVC/Non-Ethoxylated Ground Coffee Combination Successfully Treats and Prevents Metabolic Syndrome

This example describes the results of a rat diet supplemented′ with (a) non-ethoxylated ground arabica coffee and (b) ethoxylated vescalagin/castalagin rich extract (EVC) and non-ethoxylated ground arabica coffee. The diets were fed to rats having high-carbohydrate, high-fat diet-induced metabolic syndrome.

The EVC was prepared as described in Example 1. Data for various health outcomes are presented in Table 7.

TABLE 7

| | | HCHF (high | | |
|---|---|---|---|---|
| | CS | fat, high | | HCHF + |
| | (cornstarch | carbohydrate | HCHF + | coffee/ |
| Health | diet) | diet) | coffee | EVC |
| Outcomes | N = 8 | N = 10 | N = 10 | N = 9 |
| SBP* | 120.7 ± 2.4 | 143.7 ± 3.8 | 137.7 ± 1.5 | 142.6 ± 2.8 |
| (8 weeks) | | | | |
| (in mmHg) | | | | |
| SBP (12 week) | 123.5 ± 1.1 | 148.2 ± 3.4 | 132.9 ± 1.3 | 128.7 ± 3.1 |
| (mmHg) | | | | |
| SBP | 128.4 ± 1.3 | 152.8 ± 4.8 | 126.5 ± 2.1 | 121.7 ± 2.6 |
| (16 weeks) | | | | |
| (mmHg) | | | | |
| Fasting blood | 3.9 ± 0.2 | 4.7 ± 0.3 | 4.3 ± 0.1 | 4.1 ± 0.1 |
| glucose (16 | | | | |
| weeks) (mM) | | | | |
| Waist | 19.1 ± 0.7 | 23.2 ± 0.4 | 24.1 ± 0.8 | 21.9 ± 0.3 |
| circumference | | | | |
| (cm) | | | | |
| Abdominal fat | 395 ± 30 | 799 ± 57 | 788 ± 74 | 534 ± 55 |
| pads (mg/mm | | | | |
| of tibial length) | | | | |
| Whole body fat | 68 ± 6 | 155 ± 9 | 178 ± 31 | 134 ± 15 |
| mass (g) | | | | |
| Whole body | 314 ± 5 | 350 ± 5 | 310 ± 15 | 323 ± 11 |
| lean mass (g) | | | | |

*SBP—systolic blood pressure

From the data in this table, it can be seen that supplementing the diet in a rat model of metabolic syndrome with an EVC/non-ethoxylated coffee combination results in beneficial health outcomes when compared with untreated rats. Thus, EVC/non-ethoxylated coffee supplemention results in favourable health outcomes, namely
1. Treatment and prevention of metabolic syndrome
2. Lowering systolic blood pressure, back to normal
3. Lowering fasting blood glucose
4. Reducing waist circumference
5. Reducing abdominal fat pads
6. Reducing whole body fat mass
7. Increasing whole body lean mass

Supplementation with the antioxidant rich coffee produced favourable results with, respect to lowering of systolic blood pressure and fasting blood glucose. However, the EVC/non-ethoxylated coffee combination was superior in these two (2) parameters and all other aspects of metabolic syndrome.

Example 1 details the results of untreated and EVC-treated rats with high carbohydrate, high fat diet-induced metabolic syndrome. While the EVC/non-ethoxylated coffee combination does exhibit favourable health outcomes, when a comparison is made against EVC, the addition of non-ethoxylated coffee to EVC does reduce efficacy in all aspects of metabolic syndrome.

### Example 4

### Well Tolerated/High Dose EVC Ethoxylated Black Tea Successful Outcomes in Metabolic Syndrome, Cancer, Arthritis, Gout, Cholesterol, Less Exercise Induced Muscular Stiffness, Enlarged Spleen Pain, Bronchial Pneumonia Recovery, Chronic Fatigue Syndrome, Mental Acuity, Alertness in Evening, More Energy, More Motivation for Exercise, Erectile Function Improvement, Weight Loss, Reduced Chemotherapy Side Effects, Alopecia Associated with Chemotherapy

This example describes the preparation of ethoxylated oak rich black tea, wherein components of the black tea are also ethoxylated. The tea provides the equivalent of 6 g oak/1.5 g serve and is given the name EVC black tea (6 g oak/1.5 g serve).

Materials and Methods

EVC Black Tea (6 g Oak/1.5 g Serve)

Aqueous ethanol solution (4 kg, 50% w/w) with pH 3.5 was added to French oak chips (1 kg) and allowed to stand for a period of 21 days. The mixture was then filtered and the subsequent filtrate evaporated using vacuum distillation to 25% of its original volume. That is, the French oak extract (4 kg) was evaporated by vacuum distillation to give 1 kg of extract (EVC extract). The resultant de-alcoholised extract (1 kg) was derived from 1 kg oak chips, and is thus designated EVC 1:1.

The EVC 1:1 extract was added to loose leaf black tea at a ratio of 4 parts EVC 1:1 extract to 1 part black tea. This amount of extract is sufficient to saturate the tea. The EVC extract/tea leaf mixture was then dried on a stainless steel shallow tank (bed) heated to a surface temperature of about 55° C. Ambient temperature air was forced over the mixture to assist with drying, which was undertaken over a period of 2 to 3 days.

The EVC black tea (6 g oak/1.5 g serve) was tested in adjuvant-induced arthritis (AIA) rats as detailed below, with particular focus on movement disability and knee size swelling.

Rats

AIA in rats is widely used in preclinical testing of new agents for arthritis. The AIA model share's many features with human arthritis and is characterized by reliable, rapid onset and progression of a robust and easily measurable inflammation, marked bone resorption and periosteal bone proliferation (Bendele A M, ‘Animal models of rheumatoid arthritis’, J. Musculoskel. Neuron. Interact., 2001; 1(4):377-385).

Arthritis was induced in the right knee of 6-7 week old female rats by two immunisations, 7 days apart (days −21 and −14) with methylated bovine serum albumin (mBSA) (0.5 mg) in Freund's complete adjuvant containing 0.375 mg Mycobacterium tuberculosis followed by an intra-articular injection of mBSA (0.5 mg) into the right knee (day 0). Saline was injected into the left knee to serve as an internal control.

Dietary treatment with EVC black tea (6 g oak/1.5 g serve) was commenced on day −2 and continued for 16 days. Knee joint widths of both the right and left knee were measured with digital calipers daily. Gait was also assessed. A scale of 0-4 was used, whereby 0=normal movement; 1=doesn't hold up leg but limps when moving; 2=may hold leg up and when moving will use leg; 3=holding leg up all the time and when moving will use leg occasionally; 4=holding leg up all the time and when moving remains up all the time.

Results

Rats provided with EVC black tea (6 g oak/1.5 g serve) in their diet showed less restriction of leg movement post-arthritis induction when compared to untreated controls, as shown in FIG. 8. In addition, rats provided with EVC black tea (6 g oak/1.5 g serve) in their diet showed similar reductions in right knee joint width compared to untreated controls, as can also be seen from FIG. 8.

Human Testimonials

The results from human volunteers who incorporated EVC black tea (6 g oak/1.5 g serve) into their diet are detailed below.

Male (M), 54 (years old), 5 g tea/day (2 min brewing time) over a 4-week period. Prostate cancer metastasis to bone, new diagnosis, obesity. Has commenced hormone treatment, but too early to obtain an updated PSA result. Dieting, extensive exercise (20 km bike rides per day) and tea consumption has resulted in weight loss from 151 kg to 128 kg. Reported reduced muscle stiffness within 3 days of tea commencement.

M, 43, 5 g tea/day (1.5 min steeping time) over a 4-week period. Gout, abdomen pain, lethargic. Boxing coach stopped gout medication due to abdomen pain derived from a liver issue. Two weeks later started the tea and found no gout incidents in the joints. Feeling more motivated, with a more positive outlook, while on the tea. Reports more sharpness mentally.

Female (F), 43, 4 g tea/day (2 min steeping time) over a 3-week period. Lower back pain mitigated following one week tea consumption.

M, 59, 3 g tea/day (1.5 min brewing time) over a 2-week period. Arthritis pain in the fists. Reports pain relief with 1 week of tea consumption.

F, 53, 7 g tea/day (2 min brewing time) over an 8-week period. Non-Hodgkin lymphoma (B Cell lymphoma) first diagnosed in 2001. Metastasis in lymph glands. Despite very high dose, tea is well tolerated. Prior to tea consumption, treatment include successful chemotherapy and less successful stem cell replacement. Prognostic cancer marker serum lactate dehydrogenase (LDH), activity fell 6% over a 5-week period. LDH 285 H U/L (30 Aug. 2012) and LDH 268 H U/L (2 Oct. 2012). Cholesterol fell from 6.4 H mmol/L to 5.8 mmol/L. Enlarged spleen causing pain was mitigated with tea consumption. Reports feeling very well with this high dose tea consumption.

F, 42, 4.5 g tea/day (1.5 min brewing time) over a 12-week period. Tea consumption assisted in recovery from bronchial pneumonia and long-term chronic fatigue symptoms. More energy and more motivation to exercise, such as walking to and from work.

M, 45, 4.5 g tea/day (1.5 min brewing time), over a 12-week period. Generally lethargic, with aches and pain associated knee arthritis. Reported erectile function improvement. Lost 4 kg during first 6 weeks. More active at sport, followed by more alertness in the evening. Well tolerated, more energy and feeling of wellness reported.

F, 61, 7 g tea/day (2 min steeping time), 36 g/day EVC chocolate fudge (1 g oak/12 g serve), 50 g/day EVC Turmeric (4.5 g turmeric, 3.75 g oak/30 mL serve) over a 3-week period. Stage 4 ovarian cancer with lung, liver and lymph node metastasis. 2011 Taxol™ chemotherapy resulted in 3 to 5 day hospitalization following treatment and total head hair loss and fingernail cracking in 2 weeks. Cancer Biomarker CA 125 measured on 11 Oct. 2012 was 292 U/mL. Commenced the tea on 14 Oct. 2012 and Taxol™ treatment on 15 Oct. 2012. CA 125 (1 Nov. 2012) reduced to 69 U/mL considered by doctor to be an excellent outcome particularly when compared with 2011 Taxol™ treatment. No side effects experienced with this current chemotherapy, and after 3 weeks still holding a full head of hair. Noted that fingernails were not cracked as they were with 2011 treatment.

These results provide evidence for the first time that the ethoxylation of French oak, without the presence of catechins as is widely reported in the literature (e.g. Quideau S., ‘Chemistry and Biology of Ellagitannins: An Underestimated Class of Bioactive Plant Polyphenols’, World Scientific, 2009) produces superior health outcomes with surprisingly good palatability. The ethoxylation of the black tea substrate is also novel and adds to the palatability of the composition.

### Example 5

### High Dose EVC/Ethoxylated Ground Coffee Successful and Rapid Treatment of Arthritis

This example describes the preparation of ethoxylated oak rich coffee, wherein components of the coffee are also ethoxylated. The coffee provides the equivalent of 6 g oak/6 g serve and is given the name EVC coffee (6 g oak/6 g serve).

Materials and Methods

EVC Coffee (6 g Oak/6 g Serve)

Aqueous ethanol solution (4 kg, 50 w/w) with pH 3.5 was added to French oak chips (1 kg) and allowed to stand for a period of 21 days. The mixture was then filtered and the subsequent filtrate evaporated using vacuum distillation to 25% of its original volume. That is, the French oak extract (4 kg) was evaporated by vacuum distillation to give 1 kg of extract (EVC 1:1 extract).

The EVC extract was added to ground Arabica coffee at a ratio of 2 parts EVC 1:1 extract to 3 parts ground coffee. The EVC extract/ground coffee mixture was then dried on a stainless steel shallow tank (bed) heated to a surface temperature of about 55° C. for a period of 2 to 3 days. Ambient temperature air was forced over the mixture to assist with drying.

The EVC coffee was tested was tested in AIA rats as detailed below, with particular focus on movement disability and knee size swelling.

Rats

The rats and dietary treatment are as described above in Example 4.

Results

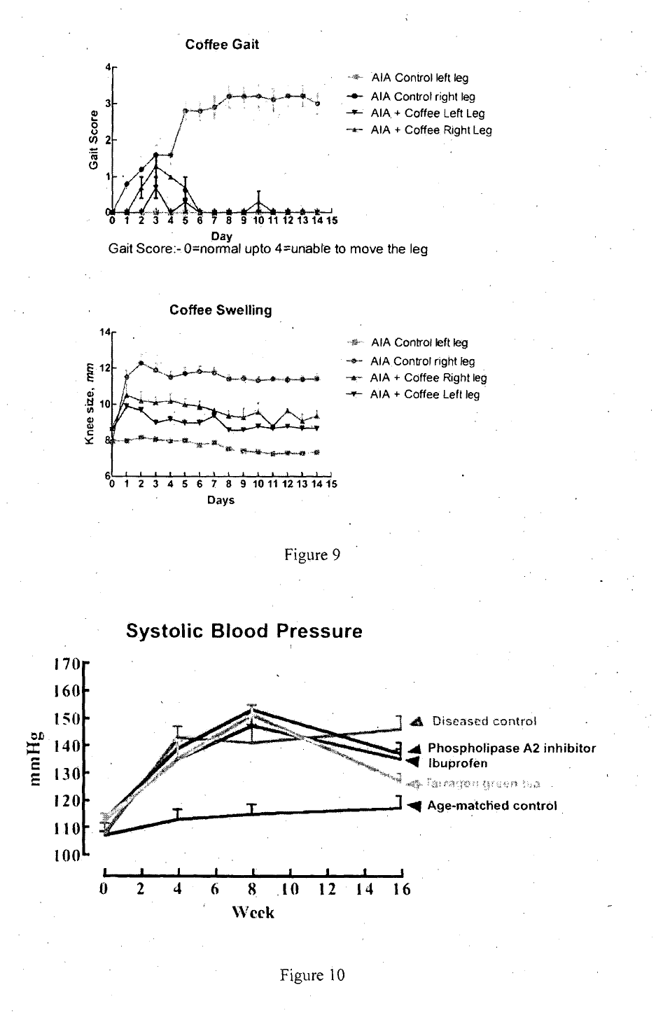

As shown in FIG. 9, rats provided with EVC coffee (6 g oak/6 g serve) in their diet showed less restriction of leg movement post-arthritis induction when compared to untreated controls. In addition, rats provided with EVC coffee (6 g oak/6 g serve) in their diet showed similar reductions in knee joint width compared to untreated controls, as can also be seen from FIG. 9.

Discussion

Both EVC coffee (6 g oak/6 g serve) and EVC black tea (6 g oak/1.5 g serve) resulted in improvement of gait and swelling in the AIA rat model. However, as can be seen from a comparison of FIG. 8 and FIG. 9, the effect of EVC coffee was significantly stronger than that of EVC black tea, even though the rats were given the same quantity of EVC 1:1 in their diet.

### Example 6

### High Dose EVC/Ethoxylated Green Tea as a Palatable Beverage Option

This example describes the preparation of ethoxylated oak rich green tea, wherein components of the green tea are also ethoxylated. The green tea provides the equivalent of 6 g oak/1.5 g serve and is given the name EVC green tea (6 g oak/1.5 g serve).

Materials and Methods

EVC Green Tea (6 g Oak/1.5 g Serve)

Preparation of EVC 1:1 extract is described in Example 4.

The EVC 1:1 extract was added to green tea at a ratio of 6 parts EVC to 1.5 parts green tea. The EVC extract/green tea mixture was then dried on a stainless steel shallow tank (bed) heated to a surface temperature of about 55° C. for a period of 2 to 3 days. Ambient temperature air was forced over the mixture to assist with drying.

The EVC green tea was taste tested.

Results

Despite the typical tannic/grassy nature of green tea and its somewhat poor ability to be a substrate for other tannic/bitter food constituents, the heavily oaked EVC green tea was surprisingly palatable. The odour and taste of oak was slightly detectable and acceptable, but with the addition of flavouring (for example, 5% terpene-based steam-distilled oil, such as lime or lemon oil) was not apparent in either taste or smell.

### Example 7

### Medium Dose EVC/Ethoxylated Black Tea Production Process Results in Natural De-Caffeination

This example describes the preparation of ethoxylated oak rich black tea, wherein components of the black tea are also ethoxylated. The black tea provides the equivalent of 4 g oak/1.5 g serve and is given the name EVC black tea (4 g oak/1.5 g serve).

Tea is pleasant tasting, well tolerated and produces successful health outcomes, namely with respect to life extension, cancer, metabolic syndrome, pain relief from bone cancer, arthritis, gout, reduction/replacement of medications, erectile function, improved libido, reduced chemotherapy, hormone treatment and radiotherapy side effects and hospitalisation, improved mobility, weight loss, cholesterol improvement, liver enzymes normalized, euphoria, feeling of jubilation, skin improvement, anxiety, mental clarity, less exercise induced muscular stiffness, mood enhancement, scrotum sac tightening, reduced the urge for coffee, calming, reduced body “fullness”, benign prostate hyperplasia, anxiety reduction, chest infection recovery, post traumatic stress disorder treatment, sleep improvement, insomnia, improved energy levels, pain and breast discomfort associate with menstruation, appetite suppression, improved work endurance, carpal tunnel syndrome, liver cysts, eyesight improvement, iritis, reduced alcohol consumption, faster recovery from colds, improved digestion, improved memory performance, stimulant and macular degeneration.

Materials and Methods

EVC Black Tea (4 g Oak/1.5 g Serve)

Preparation of EVC 1:1 extract is described in Example 4.

The EVC 1:1 extract was added to black tea at a ratio of 4 parts EVC to 1.5 parts black tea. The EVC extract/black tea mixture was then dried on a stainless steel shallow tank (bed) heated to a surface temperature of about 55° C. for a period of 2 to 3 day's. Ambient temperature air was forced over the mixture to assist with drying.

Steam distilled oil (lemon/lime, spearmint or peppermint) to be used as flavouring was sprayed onto dried EVC black tea in a rotating drum mixer for 30 minutes and then packed into foil bags. The resultant product, lemon/lime black tea, spearmint black tea or peppermint black tea is equivalent to 4 g French oak/1.5 g serve.

Results

Human Testimonials

The results from human volunteers who incorporated EVC black tea (4 g oak/1.5 g serve) into their diet are detailed below, wherein LL=lemon lime oil, SP=spearmint oil and P=peppermint oil.

M, 57, 6 g LL tea/day (30 s steeping time) over a 4-week period. Well tolerated. Drug-like euphoria, no pain after gym exercise, drank less coffee (from 8 down to 1 cup/day), no diuretic effect, fixation on purple vegetables.

M, 69, 4 g LL tea/day (1 min brewing time, however not drinking fine sediment at bottom of cup) over a period of 17 weeks. Gout, low-grade prostate cancer, Warfarin. Tea is well tolerated with Warfarin. Previously found that low dose allopurinol not working with gout issues. Replaced the allopurinol with the tea and reports no incidence of gout. Euphoria and a feeling of happiness with tea. However not noticing arthritis pain relief. Diagnosed with prostate cancer almost 7 years ago—no surgery but successful radiation treatment. PSA levels, have historically been as low as 0.1. No hormone treatment as PSA levels are low. PSA doubling over last 12 months is considered by urologist as a negative outcome, however still on “watchful waiting”. October 2012, PSA of 1.73; May 2012, PSA 1.1; November 2011, PSA 0.85.

M, 70, 87 kg, 9 g SP tea for 6 days. Used the same cup, no straining of the tea leaves. That is, previously steeped leaves remain in cup and fresh leaf added to it. Diagnosed non-metastatic prostate cancer, awaiting radical prostectomy, no treatment, no supplements. PSA doubles over a 4-month period (PSA 5 (11 Nov. 2010); PSA 5.8 (4 Apr. 2011); PSA 7.10 (5 Sep. 2011); PSA 10 (25 Jan. 2012)). On EVC black tea (4 g oak/1.5 g serve) for the period 5-10 Mar. 2012. PSA 5.4 (11 Mar. 2012) represents a halving of PSA associated with a 6-day intervention of the tea. Well tolerated.

M, 72, 3 g LL tea (2 min steeping time) for 11 days. PSA history is a doubling every 6 months. 6 months ago PSA 5.1, 11 days on the tea prior to blood test, PSA remained steady at PSA 5.7. Subsequent 6 month PSA test of 7.4 considered a good outcome by urologist. Well tolerated.

M, 55, 4.5 g SP tea for 2 weeks. Euphoria, improved erectile function. Well tolerated.

F, 61, 6 g LL tea/day (1.5 min. steeping time) over a 14-week period. Stage 4 ovarian cancer with lung, liver and lymph node metastasis. Responded well with 2011 chemotherapy (including Taxotere™, Carboplatin and Paclitaxel), with cancer antigen protein (biomarker) CA 125 reducing from about 300 U/mL to 12 U/mL. In most laboratories, the normal value is less than 35 U/mL. Patient's chemotherapy to re-commence when two conditions are met, namely (a) doubling of the CA 125 cancer marker in a 6-week period and (b) minimum of 72 U/mL reading of CA 125. Commenced tea consumption on 12 Jun. 2012 at about 3 g/day, increasing to 6 g/day after 7 days. CA 125 result is 25 U/mL (6 Jun. 2012). CA 125 result increased slowly with tea consumption to 53 U/mL (24 Jul. 2012) and then to 66 U/mL (28 Aug. 2012). Oncologist expected to start chemotherapy in August 2012, but the criteria of CA 125 reading of 72 U/mL minimum was not met. In the next 4-week period prior to the next blood test, F, 61 consumes the tea for 1 week and has no tea for the subsequent 3 weeks. During this non-compliance period the CA 125 biomarker rises 4-fold to 270 U/mL (28 Sep. 2012).

F, 52, 3 g LL tea/day (3 min steeping time) for 2 weeks. Occupation—cleaner, arthritis in hands, sore thumbs. Tea helped with hands, can make first now, can hold a vacuum cleaner. Well tolerated.

M, 55, 4.5 g tea/day (3 min steeping time). No flare-up or body pain while on the tea. Underactive thyroid, test for lupus but negative. History of testicular cancer. Well tolerated.

M, 38, 7.5 g SP tea/day (3 min steeping time) for 3 weeks. Fit, active, ex-military. Sclerosis and degenerative neck, causing pain, arthritis. Pain relief in 8 days. Stopped taking tea for 5 days, pain came back. Well tolerated.

F, 62, 4.5 g LL tea/day (3 min steeping time) for 24 weeks. Metastatic breast cancer—breast cancer with bone metastasis. Started tea on 28 Feb. 2012. Cancer marker CA15-3 was 32 U/mL (17 Feb. 2012), 26 U/mL (16 Mar. 2012), 24 U/mL (28 Jul. 2012), considered now in the safe zone. Taking salmon oil and vitamin C. No bone pain (from cancer), better mobility and reported that she felt very well while on the tea. Noted cholesterol improvement and ALT normalized. Euphoria, happier and singing frequently, while on the tea. Vivid dreams. Motivated to be physically active. Well tolerated. Mental clarity. Tired and lethargic when not on the tea for 1 week.

F, 42, 7.5 g LL tea/day (1.5 min steeping time) for 12 weeks. Tea incorporated into exercise and diet regime. Less muscle stiffness following gymnasium exercise. Lost 12 kg during a 6-week period. Put on an additional 2 kg when stopped drinking the tea during the subsequent 3-week period.

M, 71, 4.5 g LL tea/day (initially consuming tea and tea leaves, subsequent brewing practice with tea leaf discarded after 2 min brewing), for a 22-week period. Localized aggressive prostate cancer. PSA 22 (10 Jan. 2012), PSA 20 (23 Jan. 2012). Combination therapy—anti-androgen tablets (bicalutamide/Cosudex) and a luteinizing hormone-releasing hormone (LHRH) agonist (subcutaneous implant—Zoladex) began in March 2012. Radiotherapy and tea commenced in June 2012. Tea is very well tolerated when taken in conjunction with radiation treatment. Despite the side effects of hormone treatment, reports that the tea has been quite uplifting to his moods. Convinced that the combination of standard prostate cancer treatment has worked, is ecstatic with the results and his view of the future is now extremely positive.

M, 66, 3 g SP tea/day (1.5 min steeping time) for 7 months. 8 years ago, diagnosed (biopsy) with prostate cancer. No surgery, but radiation (chips) treatment. Did have TURP operation previously. Vegetarian. PSA 7.9 (January 2012), PSA 5.9 (April 2012). On tea from 18 Mar. 2012. Erectile improvement. Used to take zinc. Scrotum sac has tightened while on the tea. Helping with arthritis in hands. Feels better. Well tolerated at 2 cups/day. At early stage of administration, feeling of high blood pressure at 3 cups a day. However, now taking occasional 3 cups in 1 day.

M, 74, 3 g SP/LL tea/day for 6 months. 8 years ago diagnosed with low-grade prostate cancer (Gleeson score=6) but chose no surgery. PSA holding steady at 11.

F, 72, 3 g SP/LL tea/day for 6 months. Arthritis Australia member. Had spinal surgery but pain from spondylosis continues. After 8 weeks on the tea, able to stop medications—dextropropoxyphene (Di-gesic), codeine and oxycodone hydrochloride (Endone). Continues with paracetamol.

M, 37, 4.5 g LL tea/day in large water bottle. Drinks the sediment. Wrist pain relief in 4 days. Has calmness and feeling of wellbeing while on the tea. Doesn't have the fullness/puffiness effect typical with poor/high carbohydrate diet. Has reduced urge for coffee.

M, 72, 4.5 g LL tea/day over a 17-week period. 2002, treated for localized prostate cancer—radical prostatectomy. PSA 0.02. By 7 May 2012, PSA is 2.93. MRI and biopsy identifies small amount of prostate cancer in single lymph node as sampled by fine needle aspiration. PSA reduced to 0.02 (19 Jun. 2012) with 8 days use of tea. Continued with the tea and PSA taken September 2012, also 0.02. Pleasant tasting. Well tolerated.

M, 69, 6 g LL tea/day (30 s steeping time) over 10-week period. Improved erectile function and libido. Helps with stress.

M, 64, 3 g P tea/day over a 4-week period. Benign prostatic hyperplasia, improved urination flow with the tea.

F, 42, 4.5 g LL tea/day (consuming the tea and the tea leaves) over a 36-week period. Stage IV breast cancer, metastasized to bone and liver. Too weak for chemotherapy. With only days left to live, went on the tea. 6 months later, doctor advises that she will definitely celebrate her mid-year birthday. Now planning for Christmas period. Major quality of life improvement and extension on the tea. Estimates that extended life by 7 months. No cancer markers as a guide but reported no change in scans and in one instance, an improvement in scans.

F, 45, 3.8 g LL tea/day over a 5-week period. Metastatic breast cancer. Depressed immune system and nausea associated with radiation and chemotherapy. Chemotherapy —topoisomerase A2 inhibitors (Taxotere™, Epirubicin) and cyclophosphamide. Introduction of tea halfway through the chemotherapy cycle, results in no nausea and feeling better. Reports that 2 days were difficult with the chemo/tea combination, but still able to get out of bed and do activities.

F, 60, 3 g LL tea/day (2 min brewing time) over a 26-week period. Menopausal. Calmness, reduction in anxiety while taking the tea. Reduced back pain, even though working as a cleaner 20 hours/week, in addition to full-time sales work. Energy level good.

M, 52, 3 g P tea/day 2 min brewing time) over 2-week period. Assisted in the treatment of chest infection.

F, 73, 3 g LL tea/day (2 min brewing time) over a 4-week period. Reported a small “high” when drinking the tea and reduced anxiety levels. Well tolerated.

M, 58, 4.5 g LL tea/day (1 min brewing time) over a 2-week period. Arthritis in knee and hands. Reported first signs of pain relief in 2 days.

F, 36, 3 g LL tea/day (2 min brewing time). Suffers from anxiety disorder—post traumatic stress disorder (PTSD). Not treatable by psychologists, considered a chemical imbalance. Reported to dietitian that she experienced improved moods and sleeping better with regular tea consumption.

F, 52, 3 g LL tea/day. Suffers from anxiety disorder—post traumatic stress disorder (PTSD). Not treatable by psychologists, considered a chemical imbalance. Reported to dietitian that she experienced reduction in pain and muscle tension with the tea consumption. Sleeping better. Muscle pain and insomnia are symptoms of PTSD.

F, 39, 6 g LL tea/day (1 min steeping time) over a 3-week period. Dieting, exercising and working long hours in own business. Reported high energy levels when consuming the tea, despite a very busy lifestyle and work routine.

F, 45, 4 g LL tea/day (tea in the cup, no straining) over a 10-week period. Papua New Guinean. During the first month noticed some pressure in the head at 3 cups/day. Second month found that could easily accommodate 2-4 cups/day. Reduced menstrual pain and less breast discomfort during this period. Tends to drink 4 cups/day on weekends and is well tolerated. Weight loss from 64 kg to 61 kg. Previously size 12 in trousers, now 10. Skin condition improved. If 3 cups on an empty stomach, feeling of jubilation reported.

M, 55, 3 g LL tea/day (1 min steeping time) over a 14-week period. Healthy diet and fit for heavy work. Works a total of 65 hours per week as groundsman for 2 employers. Weight loss and strong appetite suppression while taking the tea. Wardrobe change required as a result of weight loss. Finds the tea has given him considerable energy and work endurance. Had to stop tea consumption at times, because he was losing his desire to eat.

M, 60, 3.5 g SP tea/day (brewed and consumed from a thermos flask). History of motorcycle racing accidents have now resulted in leg pain and arthritis. After 4 days consumption, reported that the very sharp pain in the leg is alleviated with a reduction in the dull ache from arthritis.

M, 73, 4 grams LL tea/day. Prostate cancer treated by intermittent hormone treatment successfully maintained at PSA at low levels (0.02). Tea is consumed as a complementary therapy.

M, 56, 4.5 g SP tea/day (1 min steeping time). Pain in knee joints and hands reduced. Able to re-commence bike rides.

M, 81, 4.5 g LL tea/day (2 min steeping time) over 4-week period. Prostate cancer metastasized to bone. While taking tea over 1 month, PSA dropped from 7.5 to 7.1 (22 May 2012). No bone pain while taking the tea. Taking Zometa™ (zoledronic acid) for complications associated with bone metastases.

M, 24, 6 g LL tea/day (1.5 min steeping time) over a 3-week period. Vivid dreams. Euphoria like endorphins. Noticed immune system felt worse when stopped taking the tea.

M, 73, 3 g LL tea/day. Prostate cancer treated with intermittent Lucrin™ treatment since 2010 resulting in PSA levels as low as 0.01. Without hormone treatment, PSA gradually increased to 0.72 (May 2012). Tea consumed during the period until next PSA test result (6 Sep. 2012) of 1.00. Urologist is pleased with result, scheduling next patient visit February 2013.

M, 74, 4 g SP tea/day. Prostate cancer. PSA 1.3 (17 Mar. 2011) rising to PSA 4.1 (23 Feb. 2012). Consuming tea for the 6-week period prior to the 18 May 2012 PSA test (PSA 4.2). Urologist pleased with the steady PSA result. Continues consumption of the tea until next PSA test result (PSA 5.6, 10 Sep. 2012).

M, 61, 2 g LL tea/day (1 min steeping time) over a 25-week period. Prostate cancer, commenced hormone treatment and tea in March 2012 (PSA 13). July 2012 PSA reading is 1.6. Currently undergoing radiation treatment. Has occasional hip ache/pain, but tea having no impact.

M, 66, 3 g LL tea/day over a 5 month period. Undiagnosed prostate condition with rising PSA. PSA 8 reduced to PSA 5.4 in 4 months while consuming the tea, as well as dandelion tea and saw palmetto.

F, 58, 3 g LL tea/day. Lung cancer primary with secondary cancer (spots) in spine. Never a smoker. Euphoric and mentally alert/positive with tea consumption. However, no pain relief. Oncologist is pleased with her overall health and appearance, considering her disease state.

M, 75, 4.5 g LL tea/day over a 4-week period. Prostate cancer with hormone treatment (goserelin acetate). Well tolerated, but reports that there is no euphoria effect.

M, 70, 2.5 g/day over a period of 20 weeks. Excellent health. No medications or supplements taken. Recent blood tests were all fine and the doctor was very pleased with a lowering of PSA levels, which was not typical for 70 year old men.

M, 83, 3 g LL tea/day for a period of 16 weeks. Prostate cancer with hormone implant recently removed due to steady PSA levels. Reports that it is a “pick me up’ in his morning schedule.

M, 51, 4.5 g SP tea/day. Muscle strain and associated pain requires the use of a girdle for support. Reported that the tea provide back pain relief within 1 week of commencing consumption.

M, 52, 4 g LL tea/day, over a period of 10 weeks. Brain cancer. Doctor reports that the latest brain scans are not showing an improvement since the patient began drinking the tea, however suggested that in this case, no changes in the scans is a positive outcome.

M, 54, 4 g LL tea/day, irregular consumption over 10 weeks. Arthritis in hands. Reports that pain relief is evident after 2-3 days consumption allowing him to participate in pastimes such as golf.

F, 56, 4.5 g LL tea/day (1.5 min brewing time) over a period of 12 weeks. Carpal tunnel syndrome. Treated with Femara™, but suffering from side effects such as feet pain. Reported pain relief within 1 week of commencement of consumption of the tea.

F, 81, 4 g LL tea/day (2.5 min brewing time) over a 13-week period. Bladder cancer. Recent scan shows a slight lesion on the bladder wall has not changed since previous scan.

M, 69, 5 g LL tea/day (1.5 min brewing time) over a 21-week period. Undiagnosed prostate condition 7.5 years ago (PSA 6) with PSA testing gradually rising with spiking over the years. PSA 8.2 (October 2011), PSA 8.7 (April 2011), PSA 9.2 (June 2012). Taking Jason Winters Tea prior to the LL tea. Did report major family-based stresses during the 2-month period of the latest PSA test period. Prostate biopsy (12 samples) in July 2012 show cancer in 2 samples and prostatic intraepithelial neoplasia (PIN) in 1 sample. Reported improvement in erectile function in the early stages of tea consumption.

F, 68, 4.5 g LL tea/day over an 8-week period. Breast cancer treated with three chemotherapy drugs. Two years suffering from arthritis in knees, some days having difficulty in walking. Reports no pain in knees following 3 days of tea consumption. The recent addition of the tea, has assisted in maintaining good energy levels while on chemotherapy. Reports euphoria and feeling great while consuming the tea. Two cysts in liver, but recent CT scan fails to detect them. Weight loss of 11.5 kg while on the chemotherapy/tea regime. Continuing with chemotherapy, but now considered cancer free.

M, 43, 4 g SP/LL tea (1.5 min brewing time) for a 13-week period. Previously required glasses to read. After 4 weeks of tea consumption eyesight improvement removed the need for reading glasses. Reports improved erectile function noted with tea consumption. Has repetitive strain injury in forearm. Found that with tea consumption, the pain was less sharp and little residual dull pain.

F, 44, 3 g LL tea/day in cranberry juice (1.5 min brewing time) over a 20-week period. Breast cancer. Treatment included minor surgery, chemotherapy, radiotherapy, bone density medication and hormone treatment inducing menopause. Despite reported side effects of weight gain post treatment, reports loss of 4 kg while taking the tea.

F, 33, 3 g LL tea/day (1 min steeping time) over 6 weeks. Inflammation of the iris (iritis). Tea consumption has “changed her life”. Prior to tea use, regular bouts of eye pain treated with corticosteroid eye drops for a period of 3 weeks following the incident. With tea use, the recent eye incident was mitigated with doubling the number of cups to 4/day over a 3 day period, resulting in less pain and discomfort in 2 days. Weight loss of 4 kg also evident while on the tea and with regular exercise.

M, 28, 3 g LL tea/day (1 min steeping time) over 6 weeks. Exercise coupled with tea consumption resulted in 4 kg weight loss in the first 4 weeks of tea consumption.

M, 56 3 g LL tea/day (1 min steeping time) over 8 weeks. Prostate cancer treated by hormone therapy. In the month of tea consumption, lost 3 kg and reduced alcohol consumption. Despite work colleagues getting colds, found that only had a sore throat for 2 days, without further symptoms.

F, 88, 2 g tea/day (1.5 min steeping time) over a 6-week period. Reports better sleep, improved digestion and memory performance while on the tea.

M, 64, 5 g SP tea/day for a period of 14 weeks. Prostate cancer with small metastasis on spine (T4). Recent treatment is hormone therapy. PSA steady at 9 and no indication of further metastases. Feels well on the tea despite the ill effects of the testosterone-lowering treatment. Well tolerated.

F, 67, 2.5 g SP tea/day for a period of 11 weeks. Knee reconstruction causing pain in recent years. Some pain relief noted with the tea in the form of dulling of pain. Considers it a morning stimulant.

M, 74, 3 g LL tea/day over a 30-week period. Prostate cancer, radical prostectomy, however in recent years PSA rising. Prior to tea consumption, PSA 4.1. With tea consumption, PSA steady at 4.2.

F, 54, 4.5 g LL tea/day over a 26-week period. Breast cancer (single breast mastectomy) with some changed lymph nodes. Successful treatment by chemotherapy (e.g. Taxotere™) and radiation. Prior to tea consumption found that some days very difficult to perform normal activity due to pain and very low energy. Tea consumption mitigated these issues. Noted that with the tea, recovered faster from chemotherapy with no extra days in hospital. No cancer markers to monitor, but noted that recent blood tests were normal, albeit low vitamin D status and the doctor was pleased with her ability to recover and be visually healthy following such intensive treatment.

F, 44, 3 g LL tea/day over a 20-week period. Suffering from anxiety (e.g. vomiting upon awakening in the morning, fatigue). Tea consumption after 2 weeks mitigated the situation. Husband reports a dramatic turnaround in her condition since taking the tea.

M, 58, 3 g tea/day over an 18-week period. Persistent tiredness. Reported that tea consumption gave a feeling of euphoria and a feeling of energy. Able to perform more hours of work in own business, attributed to the tea, in particular more alertness and energy in the evening. Some improvement in hand pain reported.

M, 88, 6 g SP/LL tea/day over a 21-week period. Prostate cancer first diagnosed in 2000. Has noticed the tea is helping with joint pain. Expect PSA test in 2013.

F, 88, 5.5 g SP/LL tea/day over an 18-week period. Age-related macular degeneration. Has a very positive attitude and good energy level while on the tea. Positive about it assisting in the macular degeneration condition, but not able to get medical verification at this stage.

F, 46, 3.5 g LL tea/day over a 16-week period. Arthritis causing hip pain, unable to work. Reported pain relief with 1 week of tea consumption. Able to return to work. Feels more positive now that tea is a regular addition to diet.

F, 53, 4.5 g LL tea/day over a 9-day period. Arthritis pain in fingers. Previously took COX inhibitor Meloxicam (Mobic™) but had to cease due to upset stomach. Each night took Panadeine Forte™ (paracetamol and codeine phosphate) to ease the pain and allow sleep. Within 4 days reported initial pain relief, with total pain relief at day 6. Well tolerated and pleasant tasting. Some improvement with facial acne. Noted that tea facilitated the passage of feces.

M, 64, 4.5 g LL tea/day over a 9-week period. Low grade prostate cancer. Vietnam veteran. Unable to have prostate cancer surgery due to disembowelment during active service in Vietnam. May 2012 PSA result 5.3. Began tea and Duodart™ on 5 Sep. 2012. PSA reduction to 2.6 (4 May 2012) considered by urologist to be very good result. M 64 is pleased with his energy levels and is very positive that his future health needs will addressed with the tea.

Caffeine Content

The EVC black tea (4 g oak/1.5 g serve) was analysed for caffeine content using HPLC against a USP caffeine standard and compared to the caffeine content of the untreated black tea.

The caffeine content of the untreated black tea was 24.02 mg/g. By contrast, the caffeine content of the EVC black tea was 18.90 mg/g. This represents a 21.3% reduction in the caffeine content of EVC black tea, which is attributed to a combination of the application of EVC extract to the tea and slow drying of the mixture of black tea and EVC extract.

### Example 8

### Medium Dose EVC/Ethoxylated Ground Coffee Production Process Results in Natural De-Caffeination

This example describes the preparation of ethoxylated oak rich coffee, wherein components of the coffee are also ethoxylated. The coffee provides the equivalent of 4 g oak/6 g serve and is given the name EVC coffee (4 g oak/6 g serve).

Coffee is pleasant tasting, well tolerated and produces successful health, outcomes, namely with respect to cancer, metabolic syndrome, blood pressure, anxiety, weight loss, arthritis, euphoria, improved platelet count (B cell lymphoma), spleen pain (B cell lymphoma), pH neutralisation, anti-depressant, menstrual pain and breast swelling, vaginal lubrication, mental acuity, euphoria, relaxing and a cold preventative.

Materials and Methods

EVC Coffee (4 g Oak/6 g Serve)

Aqueous ethanol solution (4 kg, 50% w/w) with pH 3.5 was added to French oak chips (1 kg) and allowed to stand for a period of 22 days. The mixture was then filtered and the subsequent filtrate evaporated to 25% of its original volume using vacuum distillation. That is, the French oak extract (4 kg) was evaporated by vacuum distillation to give 1 kg of extract (EVC extract) resulting in a biomass (oak) to solvent ratio of 1:1 (EVC 1:1 extract).

The EVC 1:1 extract was added to ground Arabica coffee at a weight ratio of 4 parts EVC 1:1 extract to 3 parts ground coffee. The EVC extract/ground coffee mixture was then dried on a stainless steel shallow tank (bed) heated to a surface temperature of about 55° C. for a period of 2 to 3 days until completely dry. Ambient temperature air was forced over the mixture to assist with the drying process. This ethoxylated oak/ethoxylated coffee is described as 1.33 g oak/1 g ground coffee.

The final product is a 50:50 mixture of the abovementioned oak treated coffee (1.33 g oak/1 g ground coffee) with untreated ground coffee—thus resulting in a 0.67 g oak per gram ground coffee. In practical terms, a serve of this coffee (6 g) would be prepared from 4 g dry oak, hence given the name EVC coffee (4 g oak per serve).

Results

Human Testimonials

The results from human volunteers who incorporated EVC coffee into their diet are detailed below.

M, 58, 7 g ground coffee/day over a 3.5-week period. Prostate cancer, with radiation treatment during first half of 2012. PSA readings are 1.7 (October 2011), 1.6 (February 2012), 1.7 (June 2012). During the next 4 months, no treatment but consumed the coffee for 3.5 weeks. PSA reduction to 1.04 (October 2012) considered an excellent (albeit surprising) result by urologist.

M, 53, 22 g ground coffee/day over a 14-week period. High blood pressure, obese. Blood pressure 163/93 mmHg, reduced to 137/82 mmHg and body weight 99.8 kg reduced to 94.8 kg with 14 days coffee consumption and healthy diet. Also reported reduced anxiety during this period. Muscle relaxant for damaged back (accident, 2 years prior). Pain relief for shattered first knuckle joint. Euphoria. Vivid dreams in first 2 weeks. Fist pain returned when consumption stops for 2 days. No sarcoidosis symptoms (eye flare up) during this period.

F, 66, 12 g ground coffee/day over a 10-week period. Pancreatic cancer, 16 months ago diagnosed with 3 months to live. Taste buds have changed as a result of chemotherapy for pancreatic cancer, finds EVC coffee acceptable but the LL tea unpalatable. Improvement in the cancer scan noted when on the coffee, against a background of poor scans prior to coffee consumption. Feels very well with a healthy complexion, while on the coffee. Leading a very active and fulfilling rural lifestyle.

F, 52, 16 g ground coffee/day for a 6-week period. B cell lymphoma—cancer marker CD-20 antigen positive. Enlarged spleen. Low platelet count. No medication. No pain with spleen while on the coffee. Monthly platelet count falling from 109 (27 Jan. 2012) to 60 (June 2012). One month on EVC coffee produced an increase in platelet count and no pain in the spleen. pH papers from acid to neutral.

F, 40, 17 g ground coffee/day over a 7-week period. History of depression and poor sleep patterns. Reduced anxiety with tea. Reports effective use as an anti-depressant. Reduced menstrual pain and less breast swelling. Better vaginal lubrication. When consumption ceased for 36 hours, withdrawal symptoms were evident, similar to sudden cessation of antidepressants. Found to be useful with occasional constipation issues.

M, 47, 20 g ground coffee/day for a 7-week period. Back pain managed through stretching and exercise. More alert/mental acuity when on the coffee. Experiences euphoria similar to that achieved with heavy exercise. No colds during this period despite widespread colds in workplace. Very relaxed when consumed, but not noting any energy boost.

### Example 9

### Ethoxylation Process Conditions with Caffeinated Foods Results in Caffeine Reduction and Improved Anti-Inflammatory Capability

This example describes caffeine reduction results and commensurate anti-inflammatory results achieved with EVC black tea (4 g oak/1.5 g serve) and EVC coffee (4 g oak/6 g serve) are also described.

Materials and Methods

EVC Coffee (4 g Oak/6 g Serve)

Preparation of EVC coffee (4 g oak/6 g serve) is described in Example 8.

EVC Black Tea (4 g Oak/1.5 g Serve)

Preparation of EVC black tea (4 g oak/1.5 g serve) is described in Example 7.

Results

The EVC coffee was analysed for caffeine content using HPLC against a USP caffeine standard and compared to the caffeine content of untreated coffee.

The caffeine content of untreated ground Arabica coffee was 12.60 mg/g. By contrast, the caffeine content of EVC coffee was 10.31 mg/g (prepared from equal parts of oaked coffee (1.33 g oak/1 g coffee) with a caffeine content of 8.02 mg/g and an untreated coffee with a caffeine content of 12.6 mg/g). This represents an 18.2% reduction in the caffeine content of EVC coffee, which is attributed to a combination of the application of EVC extract to the coffee and slow drying of the mixture of coffee and EVC extract.

By comparison, the addition of EVC 1:1 extract (1.33 g) extract to ground Arabica coffee (1 g) lowers the caffeine level of the resultant EVC coffee (1.33 g oak/1 g coffee) by 4.58 mg/g, equivalent to a 36.3% reduction.

An EVC black tea (4 g oak/1.5 g serve) sample has a caffeine content of 24.02 mg/g. The caffeine level of the EVC black tea was lowered by 5.12 mg/g (21.3%) compared to the caffeine level of untreated black tea.

It is postulated that the enhanced anti-inflammatory performance of EVC coffee compared to EVC black tea is linked to the formation of caffeine-vescalagin adducts and the resultant lowering of caffeine levels in the EVC coffee when compared to EVC black tea. Particularly when observing the results of consumption over up to 2 weeks, compared to over 5 days (FIG. 8 and FIG. 9), there is long-term improvement in the gait of AIA rats whose diet was supplemented with coffee compared the gait of AIA rats whose diet was supplemented with black tea.

### Example 10

### Medium Dose EVC/Non-Ethoxylated Instant Coffee is Pleasant, Well-Tolerated and Provides Successful Results in Osteoarthritis Pain Relief, Medication Replacement and a Feeling of Wellness. Also Outlined is the Production Process for EVC/Ethoxylated Instant Coffee

This example describes the successful addition of high dose ethoxylated C-glycosidic ellagitannins to a non-ethoxylated Arabica/Robusta coffee concentrate, dried into instant coffee crystals and is given the name EVC instant coffee crystals (3 g oak/2.5 g serve).

This instant coffee mixture is an example of (a) a food (e.g. beverage), (b) a food additive (e.g. ingredient for flavouring milk, chocolate manufacture, confectionary, dairy products, bakery products, and (c) a dietary supplement (e.g. chewable tablets, tablet formulas, capsules).

Materials and Methods

The preparation of EVC instant coffee crystals was as follows. EVC 1:1 was prepared as described in Example 4. EVC 1:1 (16.5 kg) was added to Arabica/Robusta coffee concentrate (55 Brix, 24 kg) and sufficient water was added to achieve a resultant mixture of 32 Brix, and the mixture was then stirred in a paddle mixer for a maximum of 10 minutes, then applied to the drying belt of a refractance window dryer. The EVC instant coffee crystals (3 g oak/2.5 g serve) was in the form, of light brown crystals (similar to the appearance of shellac flakes) that are not hydroscopic, hence can be packed in containers containing 50 g, 100 g, 150 g or larger, as is the case with other commercially available instant coffee. Alternatively the flakes can be ground to a finer size to produce a finer flake composition or a fine powder.

The taste profile of this coffee was surprisingly pleasant, with a hint of oak character. Astringency is slightly more that the untreated coffee, however not overly obvious to a consumer not skilled in the art of taste profiles associated with high polyphenol rich foods.

Where the final product is a flavoured instant coffee (e.g. caramel, hazelnut, vanilla), the addition of the flavouring can be added at any stage prior to drying, but ideally mixed as the last ingredient prior to drying.

Where the final product required is a high dose ethoxylated C-glycosidic ellagitannins on a substrate of ethoxylated coffee and a commensurately lower caffeine content, the method of manufacture is changed to allow ethoxylation of the coffee polyphenols and coupling of the natural coffee caffeine with the C-glycosidic ellagitannins to form caffeine-C-glycosidic ellagitannins adducts, whereby there is modification of the coffee polyphenols to achieve a more lipophilic character.

The preparation of EVC instant ethoxylated decaffeinated coffee crystals was as follows. Winegrape-derived ethanol (66 kg, pH 4.3, 95% v/v ethanol) was added to French oak chips (16.5 kg) and allowed to stand for a period of 20 days. The mixture was then filtered and the subsequent filtrate added to a sealable 200 L polyethylene container. Arabica/Robusta coffee concentrate (55 Brix, 24 kg) was then added to the container and the mixture stirred with a spiral mixer for 30 minutes and allowed to stand for 2 weeks at ambient conditions. The liquid mixture was then dried to a powder using refractance window drying equipment to provide a fully ethoxylated instant coffee.

In the preparation of the EVC instant coffee crystals and the EVC instant ethoxylated decaffeinated coffee crystals, sweeteners (stevia, sugar, glucose, corn syrup, honey, maple syrup), milk products (skim milk, whole milk, colostrum), vitamins, minerals, supplements (e.g. calcium, fish oil, chia seed oil, linseed oil) and other nutrients can be added and mixed into the mixture prior to the drying step.

Results

Human Testimonials

The results from a human volunteer who incorporated EVC instant coffee crystals (3 g oak/2.5 g serve) into their diet are detailed below.

M, 47, 10 g instant coffee/day over a 2-week period. Osteoarthritis. Anterior crucial ligament reconstruction, 21 year ago. Recent episodes include swelling, pain and inability to bear weight on knee. As advised by physician, on painkillers (paracetamol/codeine), 3 sets during the day, 1 set at night. After 7 days coffee consumption reported less pain and swelling, resulting in ceasing daytime medication. On second week replaced all medication. Well tolerated and feeling of wellness while on the coffee.

EVC coffee fudge (1 g oak/12 g serve) was prepared as follows. Brown sugar (550 g), butter (140 g, unsalted) and evaporated milk (375 mL) were placed into a glass bowl and microwaved on high for 3 minutes. The mixture was then stirred and microwaved for an additional 3 minutes. Chocolate (200 g, 40% cocao) was broken into pieces and added to the mixture, followed by EVC instant coffee crystals (40 g, 3 g oak/2.5 g serve) and EVC grape powder (120 g, as described in Example 12). The mixture was stirred and poured into lined, shallow trays and then refrigerated at 4° C. for 2 h. To achieve serves (squares) derived from 1 g ethoxylated French oak, the cooled EVC coffee fudge was cut using a knife into 12 g squares. The EVC coffee fudge was stored at 4° C.

The taste of EVC coffee fudge (1 g oak/12 g serve) is very acceptable, with oak character masked by the coffee flavour.

### Example 11

### Medium Dose EVC Fortified Wines are Pleasant Tasting, Well Tolerated and Produce Reduced Muscle Stiffness Associated with Exercise

The preparation of EVC red wine (2 g oak/150 mL wine) is as follows. EVC 1:1 was prepared as described in Example 4. EVC 1:1 (10 mL) was added to commercially produced oak-matured red wines derived from cabernet sauvignon, shiraz and cabernet sauvignon/shirz blends (750 mL).

Tasting of these very high dose EVC-fortified wines was surprising in that they were very palatable, albeit having obvious oak bouquet and taste. The addition did enhance the astringency (dryness) of the wine, which could be described as taking a dry red wine and converting it to a “super” dry red wine. This increased dryness was again acceptable in the taste testing (n=5). The best results were achieved with the more fruit-driven styles that include cabernet sauvignon as the main grape variety.

Noted by male (53) after 3 days of consumption (450 mL per day) that muscular stiffness/soreness was not apparent allowing for exercise on consecutive days, which was not possible prior to this EVC-fortified wine intervention.

The preparation of EVC red wine (3 g oak/150 mL wine) is as follows. EVC 1:1 was prepared as described in Example 4. EVC 1:1 (15 mL) was added to commercially produced Australian oak-matured wine derived from cabernet sauvignon, shiraz and cabernet sauvignon/shiraz blends (750 mL). The amount of ethoxylated oak extract added to 150 mL is derived from 3 g French oak.

This increased dryness' was again acceptable but 3 g added oak/150 mL red wine appears to be the approximate limit in the taste testing (n=5). Again the best results were achieved with the more fruit-driven styles that include cabernet sauvignon as the main grape variety.

The preparation of EVC white wine (2 g oak/150 mL wine) is as follows. EVC 1:1 was prepared as described in Example 4. EVC 1:1 (10 mL) was added to commercially produced Australian oak-matured chardonnay and sauvignon blanc wine (750 mL). The amount of ethoxylated oak extract added to 150 mL is derived from 2 g French oak. The wine is distinctively dry, but acceptable as a “super” dry white wine style.

However, the addition of more EVC 1:1 extract resulting in an amount of ethoxylated oak extract derived from 3 g French oak added to 150 mL white wine, is not acceptable as a table wine.

### Example 12

### Versatile EVC/Grape Powder for Beverage, Food Additive and Dietary Supplement Applications

This example describes the preparation of ethoxylated oak (ethoxylated vescalagin-rich/castalagin-rich) extract (EVC) formulated into a substrate of non-ethoxylated white grape concentrate. This mixture is given the name EVC white grape powder.

The EVC white grape powder is an example of (a) a food (e.g. concentrate added to water to make a beverage), (b) a food additive (e.g. ingredient for soft drink manufacture, wine, chocolate manufacture, confectionery, spreads, jams, dairy products, bread, biscuits, bakery products, honey, butter, cheese, margarine, meal replacement formulas) and (c) a dietary supplement (e.g. chewable tablets, tablet formulas, capsules).

The EVC white grape powder was prepared as follows. EVC 1:1 was prepared as described in Example 4. EVC 1:1 (10 kg) was added to 68 Brix white grape concentrate (20 kg) and stirred in a paddle mixer for 10 minutes and then applied to the drying belt of a refractance window dryer. The resultant dried product (16.95 kg) is hydroscopic, hence was packed in climate controlled rooms into sealed foil bags. Stored in a cooled room until required for use.

EVC toffee was prepared as follows. Maple syrup (500 mL, 100% Pure Canadian Certified Organic, Queens Fine Foods, Queensland, Australia) was heated in a saucepan, with regular stirring, until it reached 130° C. on a candy thermometer (20 minutes). The maple syrup was removed from the heat and allowed to cool to 110° C. EVC grape powder (75 g) was added with stirring. Moulds were prepared by spraying them with vegetable oil, to facilitate candy release. The maple syrup mixture was heated to 120° C., removed from the heat and poured into the prepared moulds.

EVC chocolate fudge (1 g oak/12 g serve) was prepared as follows. Brown sugar (550 g), butter (140 g, unsalted) and evaporated milk (375 mL) were placed into a glass bowl and microwaved on high for 3 minutes. The mixture was then stirred and microwaved for an additional 3 minutes. Chocolate (250 g, 50% cocao) was broken into pieces and added to the mixture, followed by EVC grape powder (200 g). The mixture was stirred then microwaved for 3 minutes. Following further stirring, the mixture was poured into lined, shallow trays and refrigerated at 4° C. for 2 h. To achieve serves (squares) derived from 1 g ethoxylated French oak, the cooled EVC chocolate fudge was cut using a knife into 12 g squares. The EVC chocolate fudge was stored at 4° C.

The taste of EVC chocolate fudge is very acceptable, with only a hint of oak character.

EVC peppermint chocolate fudge (1.5 g oak/13 g serve) was prepared as follows. Brown sugar (550 g), butter (140 g, unsalted) and evaporated milk (375 mL) were placed into a glass bowl and microwaved on high for 3 minutes. The mixture was then stirred and microwaved for an additional 3 minutes. Chocolate (250 g, 40% cocao) was broken into pieces and added to the mixture followed by EVC grape powder (300 g) and peppermint essence (25 g). The mixture was stirred, then microwaved for 3 minutes. Following further stirring, the mixture was poured into lined, shallow trays and refrigerated at 4° C. for 2 h. To achieve serves (squares) derived from 1.5 g ethoxylated French oak, the EVC peppermint chocolate fudge was cut using a knife into 13 g squares. The EVC peppermint chocolate fudge was store at 4° C.

The taste of EVC peppermint chocolate fudge (1.5 g oak/13 g serve) is very acceptable, with only a hint of oak character. The peppermint flavour is quite lingering after the confectionery is consumed.

EVC orange flavoured pectin gel (1 g oak/8.6 g serve) was prepared as follows. Citrus pectin (27 g, Grindsted™ Pectin RS 400) was dissolved in water (200 g) and allowed to stand for 10 h at 4° C. The pectin solution was placed in a saucepan and cane sugar (700 g) was added. The mixture was stirred, then allowed to boil until reduced to approximately 800 g. EVC grape powder (200 g) was then added with stirring. The mixture was heated to 90° C. and then citric acid (5 g) and orange essence (30 g) were added. The mixture was heated for a further 2 minutes, then poured into silicon moulds.

EVC Fish oil emulsion is prepared as follows. EVC grape powder (3 g) was mixed in with a serve (12 mL) of commercially available citrus flavoured fish oil emulsion (Nature's Own Omega Delight, Sanofi-Aventis, Virginia, Queensland, Australia).

The EVC grape powder dissolved very well in the emulsion. The flavour of the fish oil emulsion was sufficient to conceal the oak character of the additive.

### Example 13

### Versatile EVC/Purple Carrot Powder for Beverage, Food Additive and Dietary Supplement Applications

This example describes the preparation of ethoxylated oak (ethoxylated vescalagin-rich/castalagin-rich) extract (EVC) formulated into a substrate of non-ethoxylated purple carrot concentrate. This mixture is given the name EVC purple carrot powder.

The EVC purple carrot powder is an example of (a) a food (e.g. concentrate added to water to make a beverage), (b) a food additive (e.g. ingredient for cola/root beer/sarsaparilla flavoured soft drink manufacture, instant tea manufacture, chocolate manufacture, noodles, pasta, confectionery, spreads, jams, butter, cheese, margarine, dairy products, meat patties, sauces, marinades, honey, bread, biscuits bakery products, meal replacement formulas) and (c) a dietary supplement (e.g. chewable tablets, tablet formulas, capsules).

The EVC purple carrot powder was prepared as follows. EVC 1:1 was prepared as described in Example 4. EVC 1:1 (10 kg) was added to 60 Brix purple carrot concentrate (20 kg) and stirred in a paddle mixer for 10 minutes and then applied to the drying belt of a refractance window dryer. The resultant dried product (14.5 kg) is hydroscopic, hence was packed in climate controlled rooms into sealed foil bags. Stored in a cooled room until required for use.

### Example 14

### EVC Incorporated into Actively Growing Fruit

EVC 1:1 was prepared as described in Example 4. EVC 1:1 (10 mL) was injected 3 weeks prior to harvest and then a further amount of EVC 1:1 (10 mL) was injected 2 weeks prior to harvest. A 30 mL flavour injector was used to inject the fruit. Such injectors are cylindrical shape and comprise a piston. The needle length (6 cm) was fully inserted into an actively growing pineapple. EVC 1:1 (about 2 mL) was injected at this point with a further amount (3 mL) released when slowly pulling out the needle. This was repeated at another site, resulting in a total injection of 10 mL EVC 1:1 with the single application. Toothpicks were inserted into the 2 injection points to signal the point of entry and maintain, the integrity of the fruit.

Upon harvest the pineapple was cut up and revealed some browning of the extract where it had been applied, however there was evidence of diffusion through the plant.

### Example 15

### EVC Incorporated into Post-Harvest Fruit

EVC 1:1 was prepared as described in Example 4. EVC 1:1 (4 mL) was injected into a lemon and an orange. A flavour injector (30 mL) was used to inject the fruit as described in Example 14, resulting in two injection points, each of 2 mL of EVC 1:1. The first injection point was into the central column of the fruit. The second injection point was into the juicy vesicles area. Toothpicks were inserted into the two injection points to signal the point of entry and maintain the integrity of the fruit.

After one week the orange and lemon were cut open. There was browning of the of the central column area as the brown coloured EVC 1:1 extract was absorbed by the fibrous core. Browning also occurred in the juicy vesicle area, but was less prominent.

While this application may well be suitable for citrus that are juiced, the presentation of this fortified citrus used for eating purposes may not be ideal. An alternative option is to choose a darker flesh citrus substrate such as blood orange.

### Example 16

### EVC Incorporated into Beer Formulations to Produce Beer with Improved Taste Profiles, Well Tolerated and with Successful Health Outcomes Namely, Medication Replacement, Gout and Arthritis

This example describes the addition, of ethoxylated vescalagin-rich/castalagin-rich) extract (EVC) to commercially available beers and as an ingredient in beer manufacture. This mixture is given the name EVC beer.

EVC white grape powder preparation is described in Example 12. EVC purple carrot powder preparation is described in Example 13. EVC PC preparation is described in Example 23.

The addition of EVC white grape powder (10 g) to 50 mL pale ale (sample from 1 L, Matilda Bay Fat Yak Pale Ale, Matilda Bay Brewing Company) produced considerable foaming. After settling (about 10 minutes) the remaining 950 mL of the beer was added with stirring to produce the final product. EVC pale ale beer maintained its pleasant, full bodied taste as it presented in the untreated pale ale beer format. Evidence of the addition of the EVC 1:1 extract only slightly detectible with more hop character, slightly more flavoursome and with some additional astringency at the tip of the tongue. Overall the addition of high levels of ethoxylated C-glycosidic ellagitannins was surprisingly acceptable in beer that has no oak addition in its commercial manufacture.

10. The addition of EVC white grape powder (10 g) to 50 mL lager beer (sample from 1 L, Grolsch™, Carlton United Brewery) produced considerable foaming. After settling (about 5 minutes) the remaining 950 mL of the beer was added with stirring to produce the final product. The EVC fortified lager presented more rounded taste profiles, when compared with the untreated lager.

EVC purple carrot powder (2.2 g) was added to non-alcoholic beer (40 mL, Hopman Premium Pale, JMB Beverages). After 2 minutes, the remaining 270 mL of the beer was added with stirring. The EVC purple carrot-rich beer presented, as expected, a purple-red colour to the beer, due to the high anthocyanin content. Surprisingly there was no carrot taste in the final beer product. The taste profile was improved with the addition of EVC purple carrot by producing a more rounded mouth feel and taste. Despite the addition of the astringent oak additive, the final product was less astringent when compared with the untreated beer.

M, 50, 12 g of EVC PC/day, over an 8-week period. Gout, aggravated by excessive alcohol consumption, namely beer. Produces and consumes a home brewed lager beer (Beermakers Lager, 1.7 kg kit makes 20 L beer, manufactured by West Brew T/As Brewcraft), in accordance with manufacturers instructions. When fermented and cooled, adds EVC PC (240 g) to the batch. Has inconsistent success with Allopurinol™ over a 1.5 year period. Stops the medication. Three weeks later switches consumption from untreated Beermakers Lager (1 L per day) to EVC PC-fortified Beermakers Lager (1 L per day). Reports no incidence of gout during this 8-week period. Also notes less pain in arthritis in hands during this period. Reports that the fortified beer, while distinctively purple in colour is pleasant tasting with a slight flavour improvement over the untreated (previous) beer batches.

### Example 17

### EVC Incorporated into Onion and Garlic Powders and their Applications as a Food Additive and Dietary Supplement

This example describes the preparation of ethoxylated oak (ethoxylated vescalagin-rich/castalagin-rich) extract (EVC) formulated into a substrate of non-ethoxylated fresh onion that is subsequently gently dried to become a dehydrated form of onion with its fresh flavour preserved. This mixture is given the name EVC onion powder.

The EVC onion powder can be utilized as (a) a food additive to meat, fish, salads, cheese manufacture, hamburgers, sauces, marinades and (b) a dietary supplement (e.g. chewable tablets, tablet formulas, capsules).

The EVC onion powder was prepared as follows. EVC 1:1 was prepared as described in Example 4. The dry, outer skin of white fresh onions (30 kg) was removed and discarded. The remaining onion was put through a commercial mincer and then finely pureed in a stainless steel tilting food blender (25 L jug capacity). EVC 1:1 (10 Kg), 10 L water and the onion puree were added to a stainless steel open top vessel and a paddle mixer inserted and the mixture stirred for 15 minutes before applying to the drying belt of a refractance window dryer. The resultant dried powder was then packed into foil bags.

For production of an EVC garlic powder, the same production processes were followed as described with the EVC onion powder, however the ratio of 2 parts garlic to 1 part EVC 1:1 was used.

### Example 18

### Versatile EVC/Non-Ethoxylated Cocoa Powder for Beverage, Food Additive and Dietary Supplement Applications. Also Outlined is the Production Process for EVC/Ethoxylated Cocoa

This example describes the preparation of ethoxylated oak (ethoxylated vescalagin-rich/castalagin-rich) extract (EVC) formulated into a substrate of ethoxylated cocoa. This mixture is given the name EVC cocoa. The mixture is an example of (a) a food (e.g. drinking cocoa), (b) a food additive (e.g. cocoa ingredient for chocolate manufacture, confectionery, spreads, dairy products, bakery products, meal replacement formulas) and (c) a dietary supplement (e.g. chewable tablets, tablet formulas, capsules).

European oak wood was converted into chips and extracted for 20 days at room temperature in winegrape ethanol (pH 4.3) containing 95% v/v alcohol in the ratio of 4 parts ethanol to one part oak chips. The resultant alcoholic extract was filtered to remove the solid material and de-alcoholised in a vacuum distiller. The resultant extract is an ethoxylated vescalagin/castalagin rich extract, named EVC.

Winegrape-derived ethanol (200 kg, pH 4.3, 95% v/v ethanol) was added to French oak chips (50 kg) and allowed to stand for a period of 20 days. The mixture was then filtered and the subsequent filtrate added to a sealable 1000 L polyethylene container.

Cocoa (50 kg, Barry Callebaut Belgium NV) was gradually added and the mixture stirred in a spiral mixer for 1 hour. Hot water (200 L, 80° C.) was added to the resultant slurry and stirred continued for a further 30 minutes. The resultant cocoa/EVC liquid mixture was stored at ambient conditions for 2 weeks. The cocoa/EVC liquid mixture was dried to a powder using refractance window drying equipment to form EVC cocoa.

EVC cocoa is produced under conditions whereby the catechin-rich cocoa is ethoxylated to produce polyphenols with more lipophilic character. An alternative production process is to produce an EVC non-ethoxylated cocoa, thus retaining the integrity of the polyphenol-rich (namely epicatechin) cocoa, thus accommodating health applications such as prevention of urinary tract infections.

The preparation of EVC non-ethoxylated cocoa was as follows. EVC 1:1 was prepared as described in Example 4. EVC 1:1 (100 L) was added to cocoa (50 kg; Barry Callebaut Belgium NV) and sufficient water needed to achieve a resultant mixture of 31 Brix, and the mixture stirred in a paddle mixer for a maximum of 10 minutes, then applied to the drying belt of a refractance window dryer.

In the preparation of EVC cocoa and EVC non-ethoxylated cocoa, sweeteners such as stevia or maple syrup can be added and mixed into the slurry prior to the drying step.

EVC cocoa caramel fudge was prepared as follows. Brown sugar (350 g), butter (25 g, unsalted), milk (125 mL) were placed into a saucepan, and brought gently to the boil, with occasional stirring. After approximately 20 minutes, EVC cocoa (12 g) and caramel flavouring (3 g, Caramel flavouring V417/023441, International Flavours and Fragrances Pty. Ltd.), were added, with stirring. The mixture was then poured into a shallow silicon tray and left to set.

### Example 19

### Fractionation of EVC and its Application as a Flavouring and Dietary Supplement

This example describes the preparation of (a) a spearmint flavouring fortified with the lipophilic fraction of ethoxylated oak extract (EVC) and (b) an ethoxylated ginger flavouring fortified with EVC.

EVC Spearmint

The EVC spearmint flavouring can be utilized as (a) a flavouring for tea, chocolate, confectionery, chewing gum and dairy products (e.g. spearmint flavoured milk) and (b) a dietary supplement (e.g. chewable tablets, tablet formulas, capsules).

EVC spearmint flavouring preparation was as follows. EVC 1:1 was prepared as described in Example 4. EVC 1:1 (2.7 kg) was vacuum distilled to 270 mL and added to spearmint oil (20 mL, steamed distilled oil of Mentha spicata). The mixture was subjected to 3 minutes of high speed mixing in a domestic blender. The mixture was then stored for 12 h at 4° C. resulting in the formation of crystals on top of the brown aqueous layer. The crystals were filtered and weighed (22.4 g). These crystals were dissolved in winegrape-derived ethanol (100 mL, pH 4.3, 95% v/v ethanol), resulting in a spearmint oil fortified with the lipophilic fraction of the EVC 1:1.

EVC Ginger

The preparation of ethoxylated oak (ethoxylated vescalagin-rich/castalagin-rich) extract (EVC) formulated into a substrate of ethoxylated ginger that is subsequently gently dried to become a dehydrated form of ginger with its fresh flavour preserved is described. This mixture is given the name EVC ginger powder.

The EVC ginger powder can be utilized as (a) a food additive to meat, fish, salads, cheese manufacture, hamburgers, sauces, marinades and (b) a dietary supplement (e.g. chewable tablets, tablet formulas, capsules).

The EVC ginger powder was prepared as follows. EVC 1:1 was prepared as described in Example 4. Ginger (300 kg) was put into a commercial mincer to produce a wet ginger pulp. The ginger pulp was then placed into a winegrape hydraulic stainless steel press, to remove the ginger juice (approximately 150 kg) from the pulp. The juice was filtered then added to a vat containing EVC 1:1 (50 kg). The mixture was allowed to stand for 2 weeks, then vacuum distilled to achieve a 32 Brix product. Dried with a refractance window dryer to produce EVC ginger powder.

### Example 20

### Hydrolysed EVC Gel Production and Applications

This example describes the preparation of a water based personal lubricant that is fortified with acid-hydrolysed ethoxylated C-glycosidic ellagitannins, given the name Hydrolysed EVC gel.

The Hydrolysed EVC gel be utilized as (a) a skin active NF-κB inhibitor formulation, (b) a skin active Nitric Oxide therapy formulation, (c) a personal lubricant, (d) a condom lubricant, (e) a sexual performance enhancer/stimulant, (f) an anti-aging formulation, (g) an anti-wrinkle/skin firming formulation, (h) an anti-irritant formulation, (i) an anti-acne formulation, (j) a soothing/relaxant formulation, (l) an anti-cancer formulation, (m) an anti-inflammatory formulation, (n) an anti-hypertensive formulation, (o) an antiperspirant formulation or (p) a Rosacea formulation.

The Hydrolysed EVC gel was prepared as follows. EVC 1:1 was prepared as described in Example 4. Citric acid was added and stirred into EVC 1:1 (2 L) to achieve a homogeneous mixture with a pH of 1.8. The acidic mixture was then placed in a vacuum distiller and distilled to 100 mL over a period of 3 hours to produce Hydrolysed EVC. Hydrolysed EVC (1 mL) was mixed into a commercially available water-based gel (10 mL, Swiss Navy™ water-based lubricant, manufactured by MD Science Labs, USA) to produce Hydrolysed EVC gel.

Noted by male (53) after application of 0.5 mL Hydrolysed EVC gel to forearm skin, a minor tightening effect, but no sensation of heat. After 5 minutes, experienced slight light-headedness, but this condition subsided after 20 minutes.

### Example 21

### Suboptimal Metabolic Syndrome Results when Oak/Catechin Compete with Ethoxylated Oak Production and Implications when Coffee and Chocolate Combinations are Tested

This example describes the preparation of a non-alcoholic ethoxylated French oak/red wine extract with catechin-ellagitannin hybrids derived from red wine catechins and oak ellagitannins in the acidic wine extraction solvent (EVC CAT wine extract). Use of the EVC CAT wine extract in a diet fed to rats having high-carbohydrate, high-fat (HCHF) diet-induced metabolic syndrome is also described, as are variations whereby Arabica coffee or dark chocolate are included in the diet.

Materials and Methods

EVC CAT Wine Extract

The EVC CAT wine extract was prepared as follows. A stainless steel vessel was heated to 65° C. Chopped olive leaf (6.4 kg) was placed into an expandable cotton bag and then placed in the stainless steel vessel. French oak chips (1.5 kg) were placed into another expandable cotton bag and then placed in the stainless steel vessel. Red wine (37 L, 22% ethanol, pH 4.3) was poured over the cotton bags in the vessel and the temperature within the vessel was maintained at 65° C. for 3 days. The resultant liquid was removed from the vessel and filtered to give EVC CAT wine extract.

Rats, Diets and Test Methods Rats, diets and test methods are described in Example 1.

Coffee

Colombian-grown green Arabica coffee beans were roasted under standard industry conditions and ground so as to be suitable for a coffee plunger.

The coffee was incorporated into the HCHF diet as follows. A coffee extract was prepared by mixing ground Colombian Arabica coffee (50 g) into boiling water (100 mL). After 5 minutes, the mixture was filtered to obtain coffee extract (50 mL). The coffee extract was mixed in food by replacing 50 mL water/kg food in the HCHF diet. For each food preparation, fresh coffee extract was prepared. The rats were given free access to food and water and were individually housed in temperature-controlled conditions, with 12 h light/12 h dark.

Dark Chocolate

Dark chocolate was incorporated into the HCHF diet as follows. Callebaut dark chocolate (70% cocoa solids, Barry Callebaut Asia Pacific (Singapore) Pte. Ltd.), comprising cocoa mass (also called cocoa liquor), cocoa butter and sugar was added to the rat chow at a dose of 50 g/kg food.

Results

Data for various health outcomes are presented in Table 8.

TABLE 8

| | | | HCHF + EVC |
|---|---|---|---|
| | HCHF + EVC | HCHF + EVC | CAT wine |
| | CAT wine | CAT wine | extract + dark |
| | extract | extract + coffee | chocolate |
| Health Outcomes | N = 9 | N = 9 | N = 9 |
| SBP* (8 weeks) | 143.2 ± 4.3 | 145.0 ± 3.0 | 143.3 ± 3.7 |
| (in mmHg) | | | |
| SBP (12 week) | 132.7 ± 2.7 | 130.1 ± 2.2 | 142.7 ± 2.6 |
| (mmHg) | | | |
| SBP (16 weeks) | 124.2 ± 3.8 | 123.7 ± 2.7 | 135.7 ± 2.1 |
| (mmHg) | | | |
| Fasting blood glucose | 3.4 ± 0.2 | 4.3 ± 0.1 | 4.7 ± 0.2 |
| (16 weeks) (mM) | | | |
| Abdominal | 22.3 ± 0.4 | 22.9 ± 0.5 | 21.3 ± 0.3 |
| circumference (cm) | | | |
| Abdominal fat pads | 557 ± 30 | 600 ± 45 | 441 ± 26 |
| (mg/mm of tibial | | | |
| length) | | | |
| Whole body fat mass | 158 ± 15 | 162 ± 16 | 129 ± 9 |
| (g) | | | |
| Lean mass (g) | 304 ± 7 | 302 ± 11 | 277 ± 4 |
| Initial body weight (g) | 330 ± 5 | 330 ± 5 | 330 ± 5 |
| Final body weight (g) | 507 ± 13 | 504 ± 13 | 474 ± 9 |
| Liver weight (mg/mm | 295 ± 7 | 341 ± 18 | 278 ± 6 |
| of tibial length) | | | |

*SBP—systolic blood pressure

From the data in this table, it can be seen that supplementing the diet in a rat model of metabolic syndrome with EVC CAT wine extract results in beneficial health outcomes. Those beneficial outcomes are further enhanced by the addition of dark chocolate to the diet, notably in the abdominal circumference, abdomininal fat pads, total weight and fatty liver parameters. As can be seen from this example, the best health outcomes, with the exception of blood pressure lowering, are achieved with the combination of EVC CAT wine extract and epicatechin-rich dark chocolate.

The antioxidant-rich combination of EVC CAT wine extract and dark chocolate produced favourable systolic blood pressure results when compared with the untreated rats (Example 1), however was significantly inferior to EVC CAT wine extract alone.

Example 1 details the results of untreated and EVC-treated rats with high carbohydrate, high fat diet-induced metabolic syndrome. While the antioxidant-rich EVC CAT wine extract does exhibit favourable health outcomes, most notably systolic blood pressure lowering, when a comparison is made against EVC, EVC CAT wine extract is significantly inferior.

While there are positive references to favourable outcomes with hybrids formed between C-glycosidic ellagitannins and catechins in the literature (Quideau S, 2009), the results from Example 1 and the present example provide evidence that superior health outcomes are achieved with respect to metabolic syndrome when the oak ellagitannins are fully ethoxylated (e.g. EVC) without the presence of catechins (e.g. red wine as used in the present example).

The combination of wine-based EVC CAT wine extract and coffee consistently delivered inferior metabolic syndrome outcomes, when compared with EVC CAT wine extract alone. This is a surprising outcome and is particularly disturbing when one considers that human diets with both red wine and coffee consumption are common in the western world.

Once again, when EVC replaces the EVC CAT wine extract, as in the EVC coffee-fed rats in Example 3, the inflammation driven aspects of metabolic syndrome, such as weight gain, fat pad accumulation and fatty liver strongly favour the more anti-inflammatory EVC extract. However, the antioxidant-rich EVC CAT wine extract is comparable to the antioxidant rich EVC with respect to blood pressure reduction associated with a high carbohydrate, high fat diet.

### Example 22

### Low Dose Non-Ethoxylated American Oak/Ethoxylated Green Tea/Ethoxylated Olive Leaf Extract Formulation as a Palatable Beverage Option and Favourable Metabolic Syndrome Outcomes Superior in Respect to Hypertension and Comparable to Phospholipase A2 Inhibitor and Ibuprofen in Respect to Other Metabolic Syndrome Parameters. Human Testimonial on Fertility

This example describes the results of a rat diet supplemented with EVC CAT tarragon tea (winter tarragon, non-ethoxylated American oak+catechin-ethoxylated hybrids) and compares the results to those achieved with ibuprofen or phospholipase A2 (PLA2) inhibitor.

Materials and Methods

The diet was fed to rats having high-carbohydrate, high-fat diet-induced metabolic syndrome. Specifically, the rats were fed a diet containing condensed milk, beef tallow, fructose and powdered rat food for 16 weeks. The EVC CAT tarragon tea was added to the diet for the last 8 weeks, only, whilst the high carbohydrate high fat diet was continued.

The EVC CAT tarragon tea was prepared as follows. Ethanolic olive leaf extract (3.4 kg, pH 4.25) produced from 3.4 kg fresh olive leaf was added to green tea (3.4 kg) and left for up to 4 days to dry at ambient temperature.

Red winegrape seed/red winegrape skin extract (0.6 kg, pH 2.77), produced from 0.6 kg of frozen red winegrape pomace was added to green tea (0.6 kg) and left for up to 4 days to dry at ambient temperature.

Non-ethoxylated ethanolic American oak extract (1.8 kg, pH 5.1) produced from 0.6 kg of dry American oak chips and cane sugar ethanol (pH 6.0) was added to green tea (1.8 kg) and left for up to 4 days to dry at ambient temperature.

The resultant three antioxidant enriched green teas were blended with green tea (25 kg) to produce a green tea blend.

The green tea blend was mixed with dried winter tarragon (5 kg), Tagetes Lucinda, to produce the final product—EVC CAT tarragon tea.

The following indicators for metabolic syndrome were monitored:
(a) blood pressure (by a tail cuff procedure)
(b) heart stiffness (by a balloon in the isolated heart)
(c) abdominal fat pads (measured by waist circumference and weight of fat pads)
(d) glucose intolerance (by measuring blood glucose concentrations
(e) liver dysfunction (by analysis of liver enzymes in blood and liver histology)

Results

Rats fed the high-carbohydrate, high-fat diet showed increased blood pressure, increased heart stiffness (and increased collagen deposition), doubling of abdominal fat pads and increased waist circumference, glucose intolerance (a pre-diabetic state), increased plasma liver enzymes and deposition of fatty droplets in the liver.

Rats on the high-carbohydrate, high-fat diet but also treated with the EVC CAT tarragon tea for 8 weeks showed, at week 16, normal blood pressure, normal heart stiffness and collagen deposition, normal abdominal fat pads and waist circumference, normal glucose tolerance, normal plasma liver enzymes with no fat droplets in the liver.

The data for systolic blood pressure of rats fed a high carbohydrate, high fat diet supplemented with EVC CAT tarragon tea, PLA2 or ibuprofen is shown in FIG. 10.

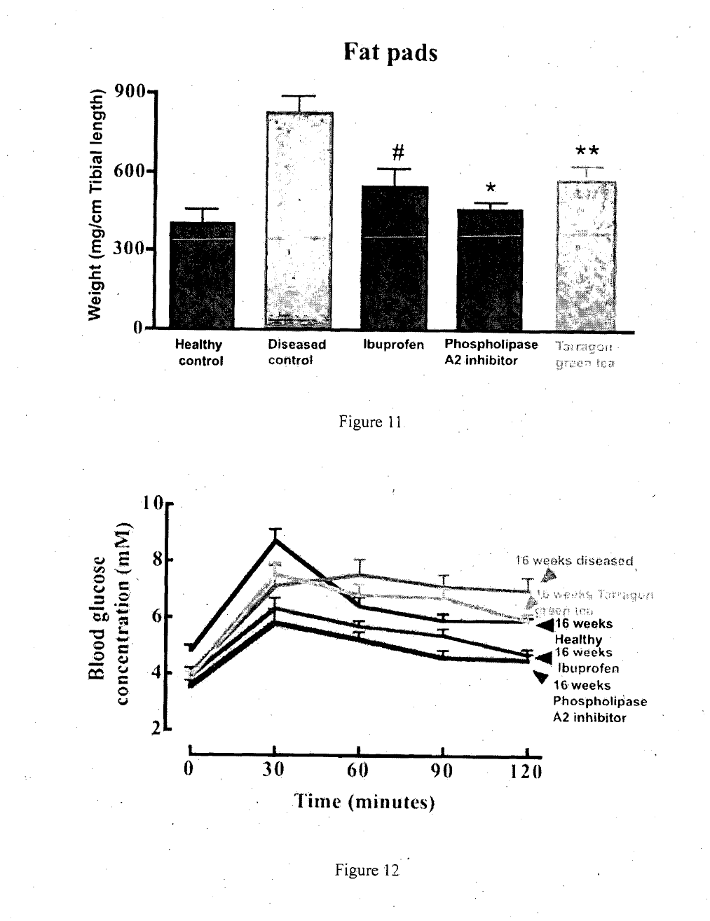

The data for fat pad measurements of rats fed a high carbohydrate, high fat diet supplemented with EVC CAT tarragon tea, PLA2 or ibuprofen is shown in FIG. 11.

The data for glucose tolerance of rats fed a high carbohydrate, high fat diet supplemented with EVC CAT tarragon tea, PLA2 or ibuprofen is shown in FIG. 12.

Human Testimonials

The results from a human volunteer who incorporated EVC CAT Tarragon tea into her diet are detailed below.

F, 28, 5 g tea/day over a 25-week period. Considered infertile. Severe back damage. Tea well tolerated and uplifting. Claims that the antioxidant rich tea assisted in her ability to conceive, whereas her history before the tea supported the view that she was unable to bear children.

### Example 23

### Low Dose EVC/Ethoxylated Purple Carrot Formulation Delivers Favourable Health Outcomes, Namely Arthritis and Migraine Prevention

This example describes the preparation of an oak enriched purple carrot powder and testing of the resultant EVC purple carrot (EVC PC) powder in an AIA rat model with particular focus on movement disability, knee size swelling and periarticular soft tissue, swelling.

The preparation of EVC PC comprised the following steps:
1. Frozen purple carrot (100 kg) was chopped up using a mincer and placed into 10 expandable cotton bags in a stainless steel vat.
2. Red wine (22% alcohol, 100 L) was pumped over the bagged purple carrot. Notably, red wine contains catechins, including epicatechin. These catechins compete with ethanol as nucleophiles in the ethoxylation process.
3. The vat was heated to 65° C. for 7 days, after which time the bags were removed and pressed to collect the liquid (about 115 L).
4. The liquid was reduced by vacuum distillation to 65 L and purple carrot concentrate (20 L, 60 Brix) and red grape concentrate (5 L, 68% Brix) were added to the reduced liquid.
5. French oak extract (10 L), derived from 2.5 kilo of dry oak extracted in 10 L of 22% alcohol red wine for 2 weeks, was added to the mixture from step 4.
6. The resultant mixture from step 5 was dried using refractance window drying to produce EVC PC powder (about 33 kg).

The EVC PC was added to the rat diet at a dose of 0.5.

Results

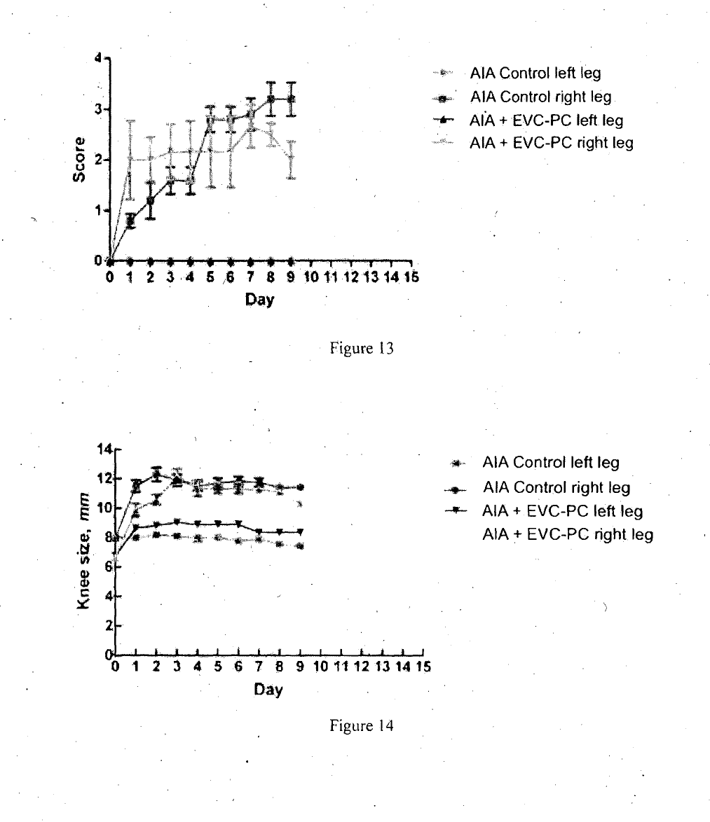

As can be seen from FIG. 13, rats whose diet was supplemented with EVC PC showed less restriction of leg movement post-arthritis induction when compared to the controls (who did not received a diet supplemented with EVC PC).

FIG. 14 illustrates that supplementing the diet with EVC PC resulted in reduction of knee joint width.

Human Testimonials

The results from a human volunteer incorporating EVC PC into their diet are detailed below.

F, 52, 9 g EVC PC/day for a 4-week period. Migraines, high stress lifestyle and work pressures. Noted that after 9 days EVC PC consumption, less tension in upper shoulder and neck. No migraines experienced during this period of consumption, unlike previous history.

### Example 24

### Low Dose EVC/Ethoxylated Purple Carrot Formulation Prevented the Increase in RANKL:OPG Ratio that Characterizes Osteoporosis

This example outlines the background and objective for the evaluation of a preventative role for C-glycosidic ellagitannin-rich EVC PC in osteoporosis. In particular, this example describes the methods and results for measurement of responses to C-glycosidic ellagitannin-rich EVC PC on osteoblasts in vitro.

Diseases with underlying inflammation are found to be associated with reduced mineral bone density and increased fracture risk; for example, obesity, type 2 diabetes, metabolic syndrome, rheumatoid arthritis, while glucocorticoids (for example, in Cushing's syndrome) are causative for osteoporosis.

Since adrenal stimulation occurs in inflammation, glucocorticoid effects may form part of the inflammation-associated response.

Homeostasis of bone mineral density is achieved by pro-mineralization actions of osteoblasts and by the resorptive actions of osteoclasts.

A crucial pathway in bone homeostasis is via the RANKL/OPG/receptor activator of NF-κB (RANK) signaling pathway involving RANK, found on the cell membrane of osteoclasts and pre-osteoclasts. RANK is activated by RANKL, synthesized by osteoblasts. RANKL-RANK interactions promote fusion of pre-osteoclasts, attachment and activation of osteoclasts, and their survival.

This potent osteoclastic mechanism is tempered by another osteoblastic product, OPG, which behaves as a “decoy” receptor by binding to RANKL, thereby limiting its action. It is generally accepted that the OPG:RANKL ratio is the predominant determinant of mature osteoclast function, as well as a potent activator of osteoclast precursors (of macrophage origin) which then fuse and differentiate into mature osteoclasts.

Pro-resorptive hormones, cytokines and growth factors act via their cognate receptors on osteoblasts to activate the RANKL/OPG/RANK pathway. The critical importance of this pathway in bone homeostasis has been demonstrated in mice gene knockouts for RANKL, OPG or RANK.

A cell model of rat osteoblast (UMR-106) that has the expected activity of mature cells, including the capacity for mineralization and the expression of RANKL and OPG was utilized.

A series of experiments (n=3) tested whether the C-glycosidic ellagitannin-rich EVC PC can prevent the increase in RANKL:OPG ratio that characterises osteoporosis.

Rat osteoblast cell line UMR-106 was stimulated with cyclic AMP for 24 hours, a treatment that increases the expression of RANKL and lowers that of OPG, thus mimicking an osteoporotic increase in RANKL:OPG.

Results

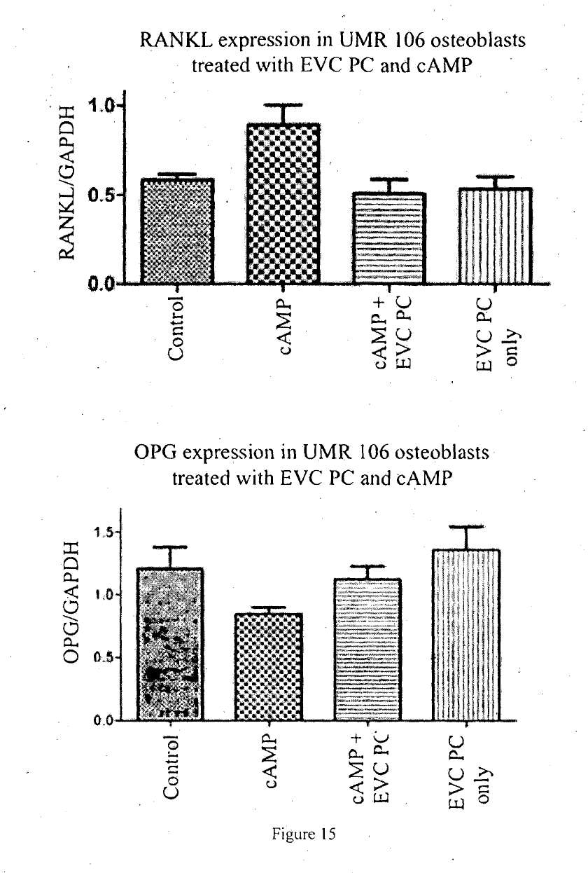

When treated concurrently with C-glycosidic ellagitannin-rich EVC PC (0.1 mM anthocyanins), the rise in RANKL:OPG was completely prevented, thus providing evidence for the use of C-glycosidic ellagitannins as an anti-osteoporotic agent, as shown in FIG. 15.

### Example 25

### Medium Dose EVC/Non-Ethoxylated Purple Carrot Wine is Pleasant Tasting, Well Tolerated and Produces Favourable Health Outcomes, Namely with Respect to Chronic Fatigue Syndrome and Arthritis

The preparation of EVC PC wine was as follows. Olive leaf extract (9.4 kg, derived from 9.4 kg fresh olive leaf), EVC 1:1 (20 kg) and purple carrot concentrate (255 kg, 58 Brix) were added to a stainless steel vat and stirred with a paddle mixer for 10 minutes and then applied to the drying belt of a refractance window dryer. The resultant dried product (164 kg) was hydroscopic, hence was packed in climate controlled rooms into sealed foil bags. Stored in a cooled room until required for wine production.

The above-mentioned dried powder was added at the rate of 40 g per 750 mL of oaked matured shiraz red wine (13% ethanol).

Results

Human Testimonials

The results from human volunteers who incorporated EVC PC wine into their diet are detailed below.

F, 39, 300 mL/day of EVC PC wine for first week, intermittent use over a 40-week period. Doctor diagnosed chronic fatigue syndrome, unable to return to work for 2 years. Tried various complementary therapy options without success. Noticed profound energy improvement in 4 days. Able to return to work 2 months later. Consumption of wine after the first 4 weeks quite irregular. Reported that when wine consumption ceased for about 1 week, there were elements of the chronic fatigue returning.

F, 29, 200 mL/day of EVC PC wine for 3-week period. Arthritis (shoulder) from sports injury. Note pain reduction with 5 days consumption and full pain relief with 1 weeks use.

### Example 26

### Medium Dose EVC/Ethoxylated Turmeric Formulation is Pleasant Tasting, Well Tolerated and Produces Favourable Health Outcomes, Namely with Respect to Cancer, Libido Improvement, Cold/Flu Avoidance, Eczema, Alopecia, Hair Re-Growth, Immune Enhancement, Reduction of “Flu Like Symptoms” with Chemotherapy and Rapid Recovery from Penicillin Resistant Infections

This example describes the preparation of a medium dose ethoxylated oak (1.5 g/30 mL serve), medium dose ethoxylated turmeric (2.5 g turmeric/30 mL serve) beverage concentrate, given the name EVC Turmeric (2.5 g turmeric, 1.5 g oak/30 mL serve abbreviated as EVCT1.

Materials and Methods

Aqueous ethanol solution (4 kg, 50% w/w) with pH 3.0 was added to French oak chips (1 kg) and allowed to stand for a period of 21 days. The mixture was then filtered and the subsequent filtrate evaporated to 33% of its original volume using vacuum distillation. That is, the French oak extract (4 kg) was evaporated by vacuum distillation to give 1.33 kg of de-alcoholised extract (designated EVC 0.75:1 extract) whereby 1 kg of this de-alcoholised EVC 0.75:1 extract is derived from 0.75 kg oak.

Fresh turmeric was placed into a commercial mincer and the resultant wet pulp was pressed in a winegrape press, separating fresh juice from pulp. The turmeric pulp was then placed into cotton bags, tied at the top and submerged into an equal weight of grape alcohol (50% ethanol, pH 3.0). After a period of at least 7 days, and preferably 9 days for extraction to be complete, the bags were then pressed in a winegrape press and the filtrate, being an alcoholic turmeric pulp extract, was vacuum distilled to 33% of its original volume and retained. Thus, 1 kg of ethanolic turmeric extract is derived from 1 kg turmeric pulp. By reducing the volume to 33% of original volume, 1 kg of this de-alcoholised extract (designated Turmeric 3:1 extract) is derived from 3 kg turmeric pulp.

The Turmeric 3:1 extract was used to prepare EVCT1 in the proportions indicated in Table 9.

TABLE 9

| Component | Proportion by weight (%) |
|---|---|
| Red grape concentrate (68 Brix) | 52 |
| White grape concentrate (68 Brix) | 27.3 |
| Blueberry concentrate (65 Brix) | 10 |
| De-alcoholised EVC 0.75:1 extract | 6.7 |
| De-alcoholised Turmeric 3:1 extract | 3.3 |
| Ascorbic acid | 0.6 |
| Sodium metabisulphate | 0.05 |
| Potassium sorbate | 0.05 |

The components in Table 9 were added in the order they are presented in the Table into a circular stainless steel vat and mixed using a low speed circulation stirrer. Stirring was continued for a further 15 minutes after the last component was added.

Results

The results from human volunteers who incorporated EVC Turmeric (2.5 g turmeric, 1.5 g oak/30 mL serve), abbreviated as EVCT1 into their diet are detailed below

M, 69, 100 mL per day. 7.5 years ago, diagnosed (biopsy) with low-grade prostate cancer. No surgery, watchful waiting protocol. PSA 9.2 (late July 2012), PSA 7.6 (27 Aug. 2012). On EVCT1 for this period. Libido improvement at the end of this period. Urologist pleased with the MRI and cancer score. Consideration of brachytherapy in the future, but continue with watchful waiting (active surveillance).

M, 56, 50 mL per day. Prostate cancer metastasized to bone (hip, 2 other hot spots), under intermittent hormone treatment. No colds since taking EVCT1 over 18 months. 2 tumour hot spots now reduced to 1.

Dog, 15 mL per day. Atopic dermatitis (eczema) and extreme hair shedding. Hair re-growth evident.

M, 72, ear skin cancer—put the sediment of EVCT1 on ear. 2 weeks later, surgeon couldn't find cancer site.

F, 45, 40 mL per day. Breast cancer. No nausea during 6-month chemotherapy, no infections. Immune system stayed well. Only 2 chemotherapy treatments resulted in “flu like symptoms”.

F, 41. Facial cancers, about to have surgery. Used EVCT1 sediment for treatment. 2 weeks and dramatic reduction in visual appearance.

F, 14, 25 mL per day. Swimmer (Representative of State of N.S.W., Australia) with history of pneumonia. No colds this season (year).

F, 47, 20 mL per day. Triple negative breast cancer. Found that the side effects of chemotherapy were negated with a daily dose of EVCT1. No colds while taking it over 18 months. Able to bounce back from penicillin-resistant infections.

### Example 27

### Low Dose EVC/Non-Ethoxylated Purple Carrot and Elderberry Combination with Favourable Arthritis Results

This example describes the preparation of an oak enriched purple carrot (EVC PC) and elderberry juice concentrate and testing of the resultant EVC PC elderberry concentrate in an AIA rat model with particular focus on movement disability and knee size swelling.

The preparation of EVC PC powder used in this example is described in Example 25. This preparation differs from the EVC PC described in Example 23, as it is produced from purple carrot concentrate and incorporates a higher concentration of EVC.

The refractance window dried EVC PC was added to elderberry juice concentrate (Brix 62) and mixed until homogeneous. The resultant EVC PC elderberry mixture was added to the dry rat food (chow) at a dose of 0.5% and elderberry concentrate (Brix 62%) was added at a dose of 5%.

Results

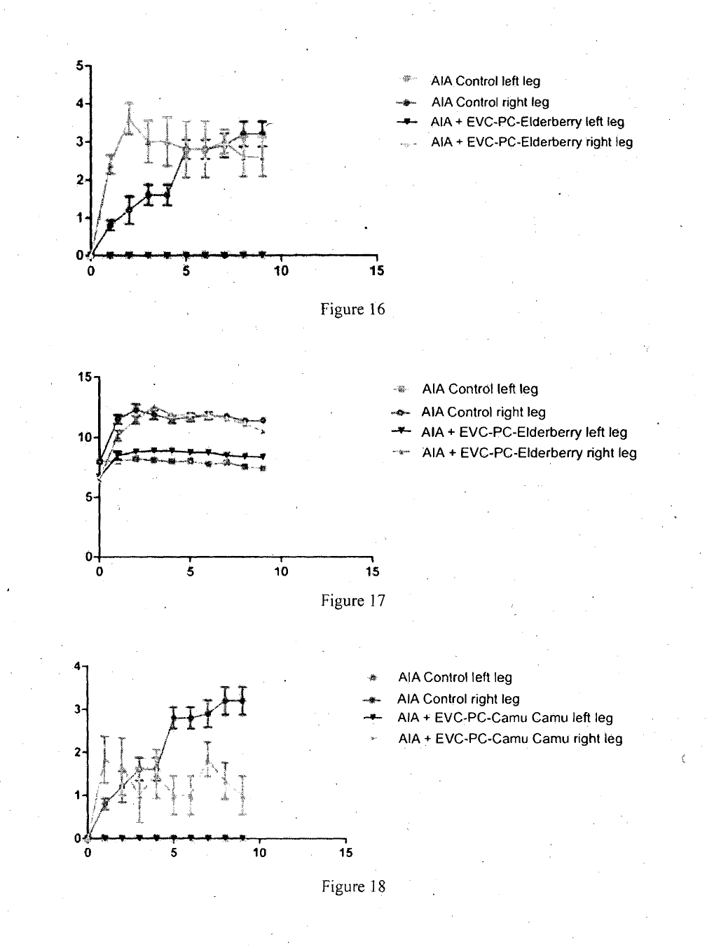

As can be seen from FIG. 16, rats whose diet was supplemented with EVC PC and elderberry showed less restriction of leg movement post-arthritis induction when compared to the controls (who did not received a diet supplemented with EVC purple carrot and elderberry).

FIG. 17 illustrates that supplementing the diet with EVC PC and elderberry resulted in reduction of knee joint width.

### Example 28

### Low Dose EVC/Non-Ethoxylated Purple Carrot and Camu Camu Combination with Favourable Arthritis Results

This example describes the preparation of an oak enriched purple carrot (EVC PC) and camu camu juice concentrate and testing of the resultant EVC PC camu camu concentrate in an AIA rat model with particular focus on movement disability and knee size swelling. The EVC PC camu camu supplement incorporates an ellagitannin-rich fruit juice (camu camu), which naturally contains ellagitannins, the fruit juice being fortified with ethoxylated oak/ethoxylated oak catechin hybrids that exist in EVC PC.

The preparation of EVC PC powder used in this example is described in Example 25. This preparation differs from the EVC PC described in Example 23, as it is produced from purple carrot concentrate and incorporates a higher concentration of EVC.

The refractance window dried EVC PC powder and camu camu juice concentrate (Brix 58) were separately added to dry rat food diet at 0.5% and 5% respectively. The addition of the EVC PC powder to the food was by mixing with water (1 part EVC PC, 10 parts water) then adding to the food. The camu camu concentrate was added undiluted to the dry rat food.

Results

As can be seen from FIG. 18, rats whose diet was supplemented with EVC PC camu camu showed less restriction of leg movement post-arthritis induction when compared to the controls (who did not received a diet supplemented with EVC PC camu camu).

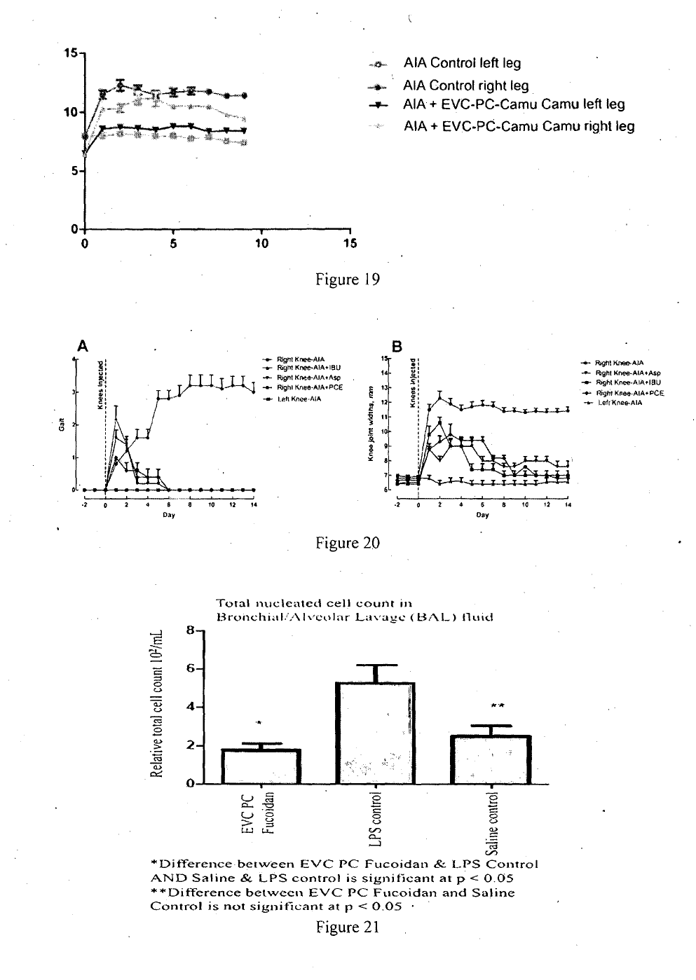

FIG. 19 illustrates that the supplementing the diet with EVC PC camu camu resulted in reduction of knee joint width.

### Example 29

### Low Dose EVC/Non-Ethoxylated Purple Carrot/Fucoidan Formulation with Excellent Arthritis Results

Materials and Methods

The preparation of EVC PC fucoidan was as follows. Olive leaf extract (4.85 kg, derived from 14.5 kg fresh olive leaf), EVC 1:1 (0.776 kg), fucoidan (3.88 kg, Synergy™, Marinova Pty Ltd, Tasmania Australia) and purple carrot concentrate (255 kg, 58 Brix) were added to a stainless steel vat and stirred with a paddle mixer for 10 minutes and then water (146 kg) was added and mixed for 5 minutes before being applied to the drying belt of a refractance window dryer. The resultant dried product (146 kg) was hydroscopic, hence was packed in climate-controlled room into sealed foil bags.

Results

As can be seen from FIG. 20A, rats whose diet was supplemented with EVC PC fucoidan showed less restriction of leg movement post-arthritis induction when compared to the controls (who did not received a diet supplemented with EVC PC fucoidan).

FIG. 20B illustrates that the supplementing the diet with EVC PC fucoidan resulted in reduction of knee joint width.

### Example 30

### High Dose Ethoxylated C-Glycosidic Ellagitannins with Purple Carrot Powder and Fucoidan

This example describes the methods used for the measurement of the effects of EVC PC fucoidan in a mouse model of lipopolysaccharide (LPS)-mediated acute lung inflammation.

Materials and Methods

The preparation of EVC PC fucoidan was as described in Example 29.

Male C57/BLK6 mice (n=8/group; 9-12 week) were fed on normal chow or normal chow supplemented with 5% EVC PC fucoidan containing interventions for 4 days. This was followed by intranasal instillation of 1.25 mg/kg E. coli (0128:B12) LPS in sterile physiological saline. Anaesthesia with 5% isoflurane in 100% oxygen was effected three hours post LPS instillation. Bronchial lavage fluid was withdrawn by standard procedures; after centrifugation, differential and total cell counts were measured and TNFα, IL-1β, IL-6 and mouse serum albumin levels in the supernatant were measured.

Results

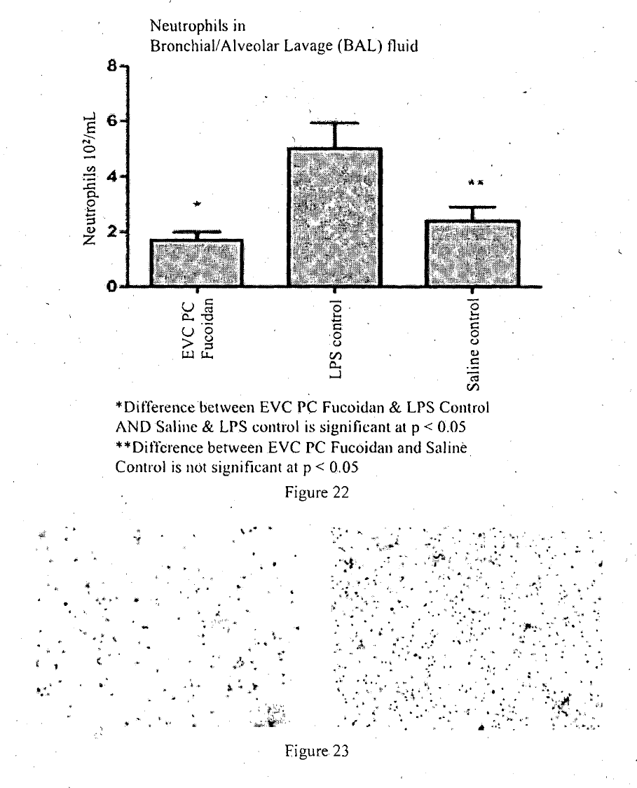

The data demonstrates that the most significant markers for acute inflammation (total nucleated cell concentration and neutrophil concentration) are significantly suppressed in EVC PC fucoidan treated mice (p<0.05; one-way ANOVA+Bonferroni's post-test analysis). These are the most common indicators used for determining effect on acute inflammation and preliminary data indicates a positive effect by EVC PC fucoidan, treatment for both parameters, as seen in FIGS. 21 and 22.

A comparison of the BAL fluid from a mouse fed EVC PC fucoidan and the BAL fluid from an LPS positive control mouse is shown in FIG. 23. Feed EVC PC 4 days prior to LPS treatment.

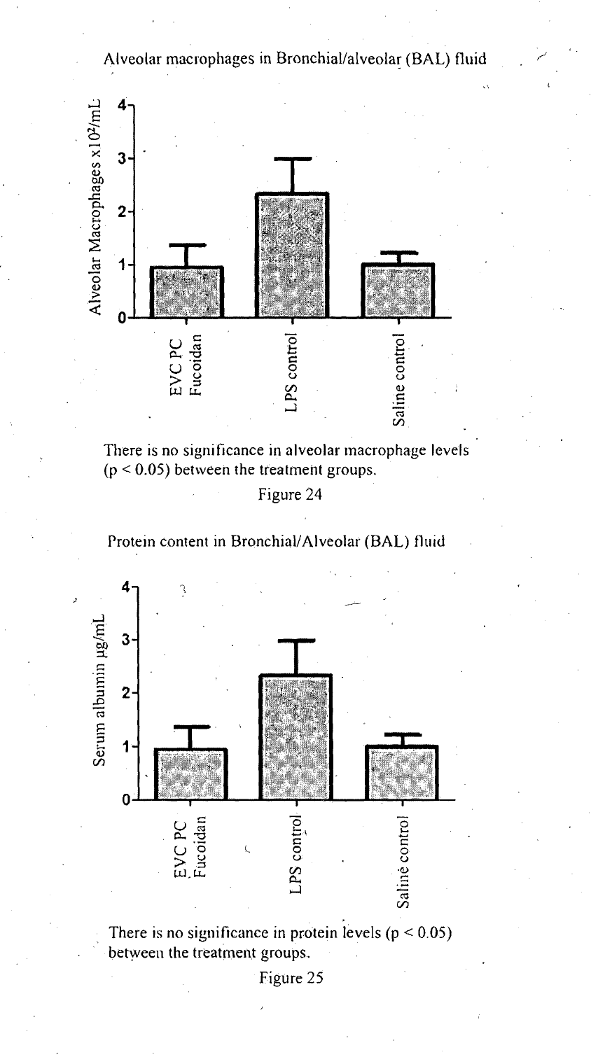

As can be seen from FIG. 24, the alveolar macrophage levels were not significantly (p<0.05) affected by EVC PC fucoidan treatment in the 3-hour assay.

However, there appeared to be a trend for depression compared to the LPS positive control. This result is not unexpected as macrophage levels normally take about 6 hours to be reliably indicative.

Protein count is an indicator of lung leak that can result in lung oedema. From FIG. 25, there was no evidence of any significant effect (p<0.05). This suggests that the mechanism of EVC PC fucoidan effect is not related to vascular leakage.

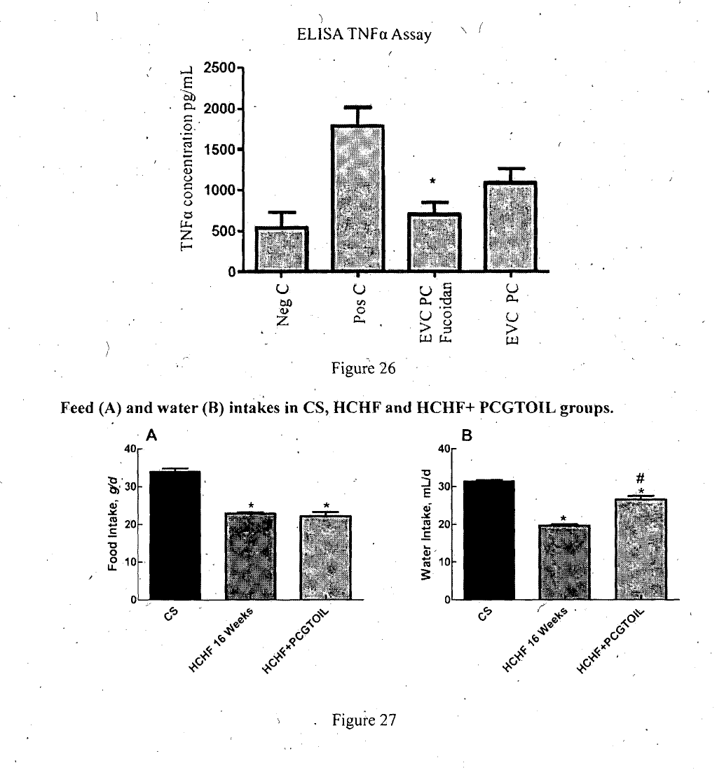

From FIG. 26, it can be seen that EVC PC fucoidan significantly depresses TNFα concentration in BAL fluid.

Discussion

There is sufficient evidence that EVC PC fucoidan significantly depresses the principle cellular markers for acute inflammation (reduction in neutrophil sequestration) by approximately 60% and also depresses TNFα concentration in BAL fluid.

There is a suggestion that EVC PC fucoidan treatment may also suppress alveolar macrophage levels. EVC PC fucoidan does not appear to affect lung leak. However, it appears that EVC PC fucoidan has a significant effect on these two indicators of markers for acute inflammation in the lung.

Preliminary measurement of the effects of EVC 1:1 extract (preparation is described in Example 4) in this same mouse model of lipopolysaccharide (LPS)-mediated acute lung inflammation, most notably IL6 results of bronchial lavage fluid, are confirming that the EVC component of the EVC PC fucoidan′composition is having a significant contribution to these successful lung inflammation results.

Human Testimonials

The results from human volunteers who incorporated EVC PC fucoidan into their diet are detailed below.

M, 36, 4.5 g EVC PC fucoidan per day over a 52-week period. Hypertriglyceridemia. Nine years ago, diagnosed with very high triglyceride levels in the blood (9.9 mmol/L). Dietician proposes and assisted with dramatic dietary change and lower alcohol consumption that brought about a lowering of triglyceride to 5.4 mmol/L. Subsequent medication (fenofibrate) brought levels down to 3.9 mmol/L. Stopped taking the medication and after 4 months began consuming the EVC PC fucoidan for a year, resulting in a recent level of 2.2 mmol/L. While still considered in a high range, the results are very pleasing for the consumer and his doctor. It should be noted that diet and alcohol consumption when taking the EVC PC fucoidan was very poor compared with the dietician administered diet 4 years ago.

### Example 31

### Suboptimal Metabolic Syndrome Results (Notably Abdominal Fat Reduction) when Oak/Green Tea Catechin Hybrids Compete with Ethoxylated Oak Production in this Formulation that Also Incorporates Omega 3 Oil

This example describes the preparation of an oak enriched purple carrot/green tea/linseed oil (PCGTOIL) combination. The effect of the PCGTOIL combination in diet-induced metabolic syndrome in rats, in particular, the effects on cardiovascular and metabolic symptoms are assessed.

Materials and Methods

PCGTOIL was prepared as follows.
1. Frozen purple carrot (100 kg) was chopped up using a mincer and placed into 10 expandable cotton bags in a stainless steel vat.
2. Red wine (22% alcohol, 100 L) was pumped over the bagged purple carrot. Notably, red wine contains catechins, including epicatechin. These catechins compete with ethanol as nucleophiles in the ethoxylation process.
3. The vat was heated to 65° C. for 7 days, after which time the bags were removed and pressed to collect the liquid (about 115 L).
4. The liquid was reduced by vacuum distillation to 65 L and purple carrot concentrate (20 L, 60 Brix) and red grape concentrate (5 L, 68 Brix) were added to the reduced liquid.
5. French oak extract (10 L), derived from 2.5 kg of dry oak extracted in 10 L of 22% alcohol red wine for 2 weeks, was added to the mixture from step 4.
6. The resultant mixture from step 5 was combined in a stainless steel vat with dried orange carrot juice powder (7 kg, Nutradry, Queensland Australia), dried beetroot juice powder (7 kg, Nutradry, Queensland Australia), linseed oil (8 kg, Proteco Queensland) and green tea extract (3 kg hot water extract derived from 0.5 kg dry green tea), mixed for 10 minutes using a paddle mixer and then dried using refractance window drying to produce PCGTOIL.

Rats were fed an HCHF diet for 16 weeks. The HCHF consisted of fructose (175 g), powdered rat food (155 g), beef tallow (200 g), condensed milk (395 g), Hubble, Mendel and Wakeman salt mixture (25 g) and water (50 ml) per kilogram of food. The drinking water was augmented with 25% fructose in the water. For the control diet, fructose and condensed milk were replaced with corn starch (575 g) and beef tallow was replaced with water (200 ml). After 8 weeks for the following 8 weeks, the diet was supplemented with PCGTOIL (2% of the diet). Rat body weight, food and water intakes were measured daily. Waist circumference, systolic blood pressure and oral glucose tolerance were measured every four weeks. After euthanasia at 16 weeks, organ weights (including abdominal fat depots) and heart function (isolated Langendorff heart) were measured. All data are presented as mean±SEM; n=6-8 for each group. The statistical significance was tested with standard procedures and shown as *P<0.05 vs. corn starch; #P<0.05 vs. HCHF.

Results

The PCGTOIL intake was 0.6±0.03 g/day. Addition of PCGTOIL to the HCHF diet resulted in reduced abdominal fat pads and circumference as measures of visceral adiposity. Cardiovascular parameters such as blood pressure, heart weight and left ventricular stiffness were also normalized, and glucose tolerance was improved.

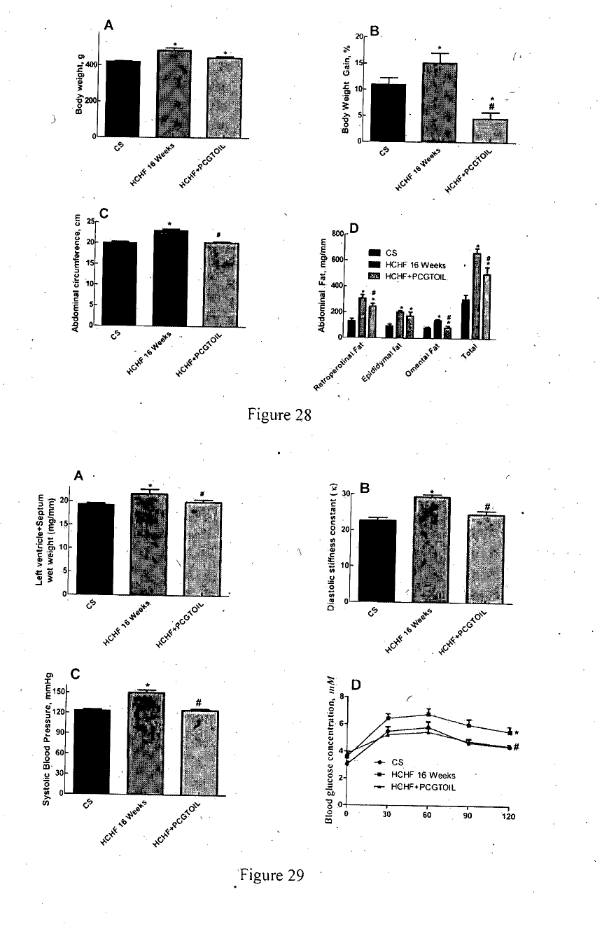

Data for feed and water intakes for rats on a cornstarch (CS) diet (control), HCHF diet or HCHF+PCGTOIL are shown in FIG. 27. Data for body weight, % body weight gain, abdominal circumference and abdominal fat deposition in rats fed a cornstarch (CS) diet (control), HCHF diet or HCHF+PCGTOIL are shown in FIG. 28. Data for left ventricular weight, left ventricular stiffness, systolic blood pressure (SBP) at 16 weeks and oral glucose tolerance for rats on a CS diet, HCHF diet or HCHF+PCGTOIL are shown in FIG. 29.

### Example 32

### Suboptimal Metabolic Syndrome Results when Oak/Green Tea Catechin Hybrids Compete with Ethoxylated Oak Production in this Instant Tea Formulation

This example describes the preparation of an ethoxylated extract of oak and green tea (EVC CAT wine tea) mixed with fucoidan-rich seaweed extract, flavoured with lemon and lime oils, then dried using RWDP to make an instant tea. The extraction medium (22% alcohol red wine) contains catechins (for example, epicatechin) and the mixture includes catechin-rich green tea. Specifically, the acid-catalysed ethoxylation conditions include the presence of catechin-rich polyphenols that compete with the oak ethoxylation process that yields EVC.

Ethoxylated green tea extract was prepared by the addition of green tea leaves to red wine (22% ethanol) at pH 3.5 in a biomass (green tea) to solvent ratio of 1:4. The resultant slurry was allowed to stand at room temperature for 1 hour. The mixture was filtered to remove any solid material, and the filtrate was retained. This product was vacuum distilled to achieve 40 Brix.

EVC CAT wine extract preparation is described in Example 21. This product was vacuum distilled to achieve 40 Brix.

The bladderwrack seaweed extract powder (Marinova Pty Ltd, Maritech® Synergy) has a minimum of 80% fucoidan content.

The lemon and lime oils are steam distilled food flavourings. The components were combined in the proportions listed in Table 10.

TABLE 10

| Component | Proportion % (w/w) |
|---|---|
| Ethoxylated green tea extract | 37 |
| EVC CAT wine extract | 57.6 |
| Lemon oil | 1.6 |
| Lime oil | 1.6 |
| Maritech ® Synergy Fucoidan powder | 2.2 |

The components listed in Table 9 were mixed using a high speed mixer and the resultant mixture was dried using RWDP. The resultant dried product was very hydroscopic, hence was packed in climate controlled rooms into sealed foil bags. Stored in a cooled room until required for use.

This example also describes the attenuation of cardiovascular and metabolic symptoms of metabolic syndrome in rats.

Materials and Methods

Rats were fed a high carbohydrate/high fat (HCHF) diet for 16 weeks. The HCHF diet consisted of fructose (175 g), powdered rat food (155 g), beef tallow (200 g), condensed milk (395 g), Hubble, Mendel and Wakeman salt mixture (25 g) and water (50 mL) per kg of food. The drinking water in the HCHF-fed rats was augmented with 25% fructose in the water. For the control diet, fructose and condensed milk were replaced with corn starch (575 g) and beef tallow was replaced with water (200 mL). After 8 weeks for the following 8 weeks, the diet was supplemented with seaweed extract (0.5 g/kg food) and lemon lime tea with seaweed extract (2.7 g/kg food). Rat body weight, food and water intakes were measured daily. Waist circumference, systolic blood pressure and oral glucose tolerance were measured every four weeks. After euthanasia at 16 weeks, organ weights (including abdominal fat depots) and heart function (isolated Langendorff heart) were measured.

Results

All data are presented as mean±SEM; n=6-8 for each group. The statistical significance was tested with standard procedures and shown as *P<0.05 vs. corn starch; #P<0.05 vs. HCHF.

The intake of seaweed extract was 11.7±0.5 mg/day and the intake of lemon lime tea with seaweed extract was 79.7±5.0 mg/day.

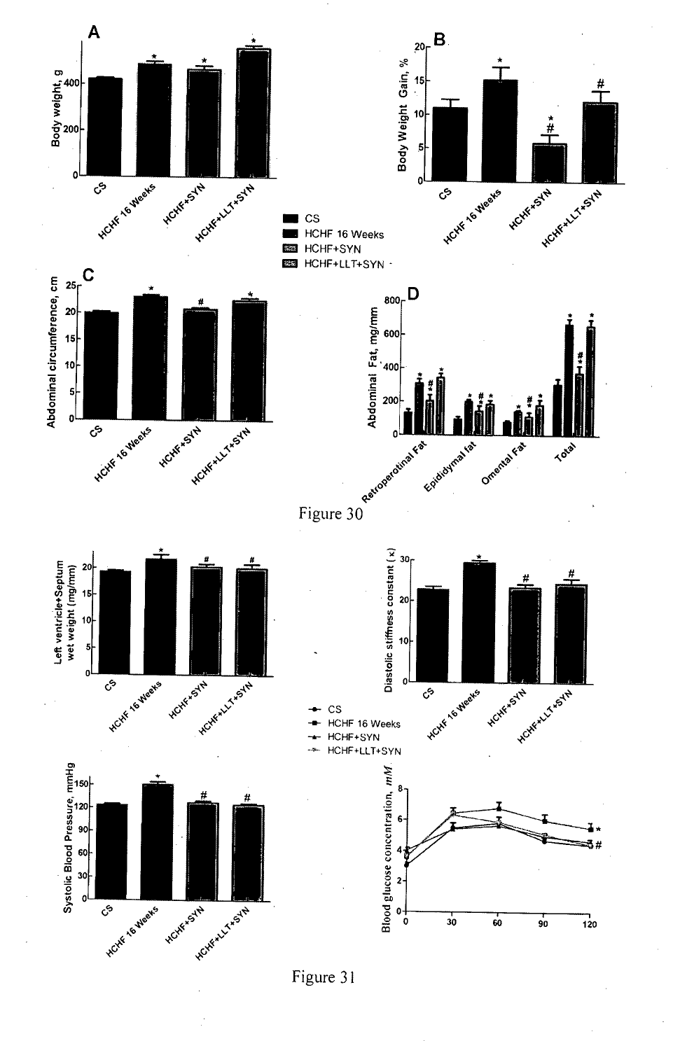

As can be seen from FIGS. 30 and 31, addition of the seaweed extract to the high carbohydrate, high fat diet in rats reduced abdominal fat pads and circumference as measures of visceral adiposity, normalized cardiovascular parameters such as blood pressure, heart weight and left ventricular stiffness and also improved glucose tolerance.

Addition of lemon lime tea containing seaweed extract decreased the cardiovascular parameters and improved oral glucose tolerance. However, the lemon lime tea with seaweed extract had no effect on abdominal fat deposition compared to rats fed the high carbohydrate, high fat diet only, and body weight increased more than with the high carbohydrate, high fat diet alone.

While there are positive references to favourable outcomes with hybrids formed between C-glycosidic ellagitannins and catechins in the literature (Quideau S, 2009), the results from Example Land this example provide evidence that superior health outcomes are achieved with respect to metabolic syndrome when the oak ellagitannins are fully ethoxylated (e.g. EVC) without the presence of catechins (for example red wine and green tea used in Example 32).

### Example 33

### Suboptimal Cancer Results when Oak/Green Tea Catechin Hybrids Compete with Ethoxylated Oak Production in this Instant Tea Formulation

This example describes the materials and methods used to measure cancer cell numbers in response to EVC CAT wine tea.

EVC CAT wine tea was prepared as described in Example 31

Cell Culture and Preparation for Testing Efficacy of EVC CAT Wine Tea

Bone metastasised androgen receptor negative prostate cells (PC-3; CRL-1435; American Type Culture Collection, Manassas, Va., USA) were grown in Roswell Park Memorial Institute (RPMI) 1640 media supplemented with 10% fetal calf serum (Hyclone), penicillin (100 units/mL) and streptomycin (100 mg/mL).

The PC-3 cell line was cultured at 37.8° C. in an incubator providing a humidified environment in the presence of 5% CO₂ and 95% air.

Treatment Protocol for Testing Efficacy of EVC CAT Wine Tea

Exponentially growing cells were cultured in 96-well plates for 24 hours before treatment with EVC CAT wine tea. EVC CAT wine tea was first diluted to 1% (w/v) in culture medium and sterilised by filtration. Further dilutions were made in the medium to achieve lower concentrations. After 72 hours of treatment time, the cells were harvested and SYBR green was used to measure the cell numbers. Untreated cells served as controls.

Results

After 72 hours of treatment time, the PC-3 cells were round in shape, but were still attached to the bottom of the plate. Whilst 0.1% of the products inhibited cell growth, it did not appear to kill the cells.

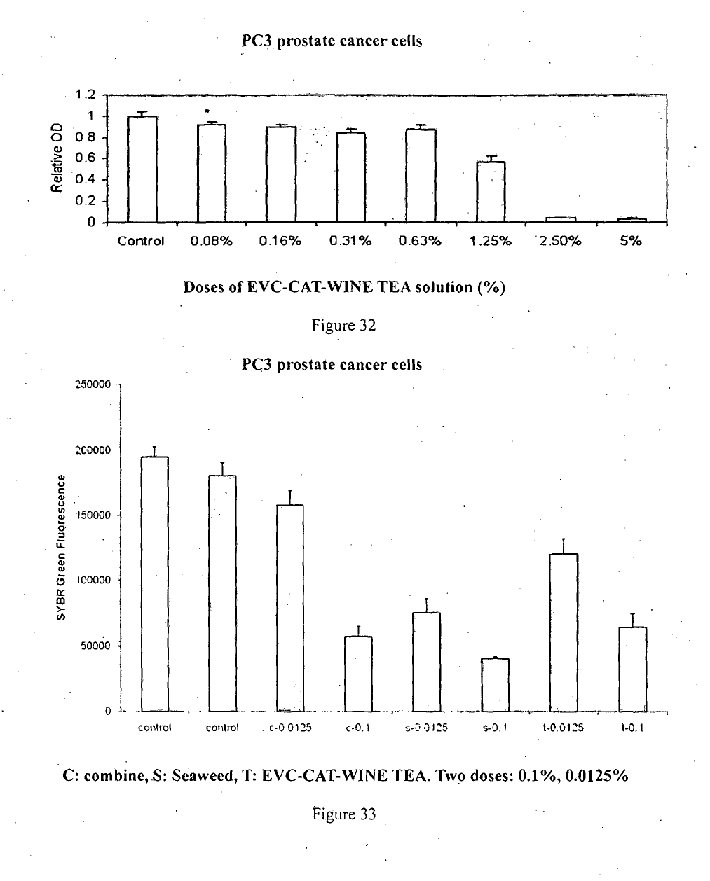

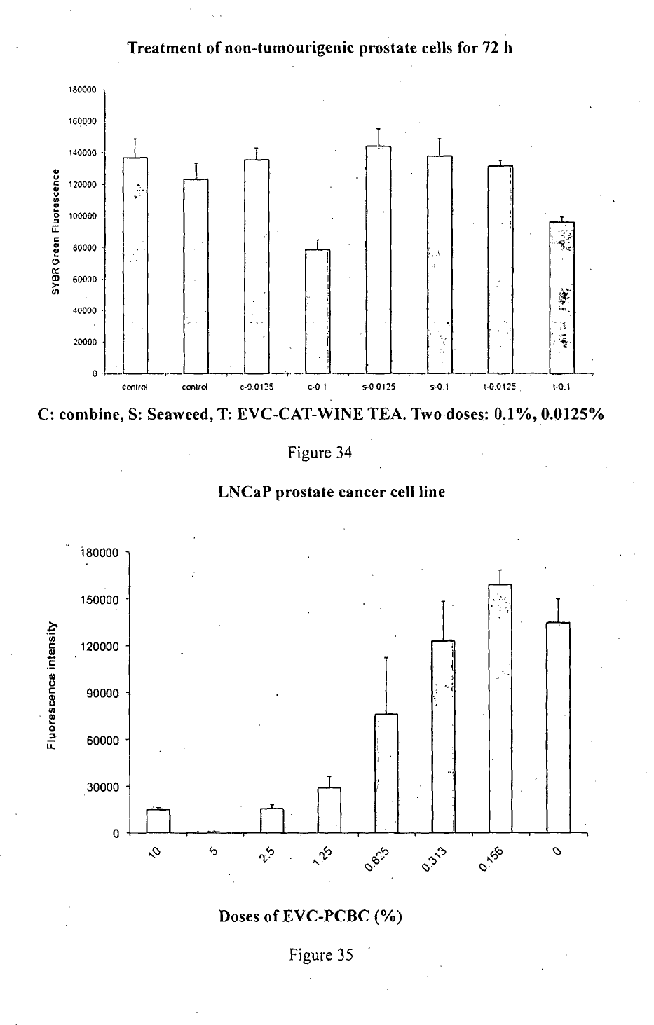

The resultant data are depicted graphically in FIGS. 32 to 34. Specifically, FIG. 32 illustrates the effect of EVC CAT wine tea extract on the PC-3 prostate cancer cell line. FIG. 33 illustrates the effect of EVC CAT wine tea extract, seaweed extract and combined extracts of EVC CAT wine tea and seaweed on the PC-3 prostate cancer cell line, and FIG. 34 illustrates the effect of EVC CAT wine tea extract, seaweed extract and combined extracts of EVC CAT wine tea and seaweed on non-tumourigenic prostate cells.

The hybrids formed between C-glycosidic ellagitannins and grape flavan-3-ols (acutissimins) have been described in previous art as having favourable results in cancer testing (Quideau S, 2009). However, Example 32 illustrates poor results achieved when procedures used encourage the formation of acutissimins, as is the case with EVC CAT wine tea.

Further support to the inferior performance of foods that encourage acutissimins production at the expense of ethoxylated C-glycosidic ellagitannins is that no positive results were reported with 33 volunteers who consumed EVC CAT wine tea as a treatment of prostate cancer, breast cancer, weight loss or arthritis.

These poor results with EVC CAT wine tea are further emphasized when compared with the teas described in Example 4 and Example 7, whereby the C-glycosidic ellagitannins are ethoxylated without the presence of flavan-3-ols or tea catechins, resulting in very strong in vivo arthritis results and volunteer testimonials report excellent results with the treatment of prostate cancer, treatment of breast cancer, weight loss and arthritis.

In contrast to the poor EVC CAT wine tea results, fucoidan (SYN) results were positive in respect to cancer cell numbers without exhibiting toxic effects to non-tumourigenic prostate cells.

### Example 34

### Medium Dose EVC/Ethoxylated Turmeric Formulation is Pleasant Tasting, Well Tolerated and Produces Favourable Health Outcomes, Namely Cancer, Pain Associated Bone Cancer, Cold Sores, Ulcers, Improved Moods and Dementia

This example describes the preparation of a high dose ethoxylated oak (3.75 g/30 mL serve), high dose ethoxylated turmeric (4.5 g turmeric/30 mL serve) beverage concentrate and is given the name EVC Turmeric (4.5 g turmeric, 3.75 g oak/30 mL serve), abbreviated as EVCT2.

EVC 0.75:1 extract and Turmeric 3:1 extract preparations are described in Example 26.

EVC PC preparation is described in Example 23.

The filtrates were combined in the proportions as shown in Table 11, to give EVC Turmeric (4.5 g turmeric, 3.75 g oak/30 mL serve).

TABLE 11

| Component | Proportion by weight (%) |
|---|---|
| Purple carrot concentrate (60 Brix) | 59.3 |
| Blueberry concentrate (65 Brix) | 15 |
| De-alcoholised EVC 0.75:1 extract | 10 |
| De-alcoholised Turmeric 3:1 extract | 5 |
| EVC PC powder | 10 |
| Ascorbic acid | 0.6 |
| Sodium metabisulphate | 0.05 |
| Potassium sorbate | 0.05 |

The components were added in the order they are presented in the Table into a circular stainless steel vat and mixed using a low speed circulation stirrer. Stirring was continued for a further 15 minutes after addition of the potassium sorbate.

Results

The results from human volunteers who incorporated EVC Turmeric (4.5 g turmeric, 3.75 g oak/30 mL serve) (EVCT2) into their diet are detailed below.

M, 74, 100 mL per day. Prostate cancer. PSA is steady. Considerable pain reduction with EVCT2. Used to take 6 Panadiene Forte™ per day.

M, 80, 75 mL per day. Metastatic prostate cancer. January 2009 advised only 12 month to live. Commenced EVCT2 in 2010 and also was 1 of 1088 prostate cancer patients in 12 countries participating in the Zytiga™ (abiraterone acetate) trial. In the Zytiga™ group, the cancer progressed at only half the speed as the control group, with patients reporting significantly less pain and a noticeable delay before they had to undertake chemotherapy. EVCT2 patient showed similar response in the 12-month trial (no pain) and was considered the “leader of the group” based on tests, hence oncologist decided to continue with the same “treatment” for a further 1.5 years. After 2.5 years on the trial it was discovered that the patient was on a placebo.

F, 60, 100 mL per day. Breast cancer metastasized to bone. 50 mL diluted 1:1 in water. Held in mouth for 1 minute before swallowing. Cancer marker (CA 15.3) 86 to 36 in 18 months.

M, 71, 40 mL per day. Prostate cancer with lymph metastasis. PSA reduction with EVCT2.

F, 52, 20 mL per day. Suffering mouth cold sores/ulcers for a few months. Problem reduced within 4 weeks use of EVCT2.

M, 81, 25 mL per day. Dementia—9 years. Maintains good moods, no temper or aggression while on EVCT2. Carer nurse reports that he has not deteriorated like other patients. Nurse considers that it has slowed down the disease progression.

### Example 35

### Medium Dose EVC/Non-Ethoxylated Food Extract Formulation Produces Significant Results in Cancer Re-Occurrence In-Vitro/Human and Favourable Health Outcomes in Cancer, Life Extension, Dementia, Aggression Associated with Dementia, Cold Sores, Ulcers and Pain Associated with Bone Cancer

This example describes the testing of EVC purple carrot blueberry concentrate (EVC-PCBC) on non-synchronised prostate and breast cancer cell lines.

Materials and Methods

This example describes the preparation of ethoxylated vescalagin/castalagin rich extract (EVC), ethoxylated winegrape extracts and non-ethoxylated food extracts into a coloured substrate (CS) base of purple carrot vegetable juice concentrate and blueberry fruit juice concentrate, named EVC purple carrot blueberry concentrate (EVC-PCBC).

EVC-PCBC is a blend of four components, namely (a) ethoxylated oak aged winegrape extract (ethoxylated vescalagin/castalagin rich extract (EVC)), (b) ethoxylated grape seed/skin extract, (c) non-ethoxylated food pulp extracts (ginger, turmeric, citrus skins and green tea), (d) on a coloured substrate (CS) base of purple carrot concentrate and blueberry juice concentrate.

Grape brandy (5 kg, 62% v/v) with pH 3.5 was added to European oak chips (1 kg, Quercus Robur/Quercus petraea L.) and allowed to stand for a period of 2 months at room temperature. The mixture was then filtered to remove the solid material and the filtrate, being an ethanolic EVC extract, was retained. This ethanolic extract is named EVC 1:4 extract.

An extract of red wine grape skins and seeds, which is a byproduct of a winemaking process, and therefore comprises ethoxylated components was added to ethanol (96% v/v, derived from cane sugar) at pH 6 in a 1:1 biomass:solvent ratio. The resultant solution had a pH of 4.5 and was allowed to stand at room temperature for 2 to 3 weeks. The mixture was filtered to remove any solid material, and the filtrate was retained.

Non-ethoxylated turmeric and ginger extracts were prepared by placing whole fruit in a juice extractor. The resultant pulp/skin was added to an equal weight of ethanol (96% v/v, derived from cane sugar) at pH 6 in a 1:1 ratio for about 3 weeks at room temperature. The mixture was filtered to remove any solid material, and the filtrate was retained.

Non-ethoxylated citrus skin/pulp were prepared individually from whole citrus fruit (orange, lemon, mandarin, Tahitian lime, kaffir lime) processed through a commercial juice extractor. The resultant pulp/skin (biomass) was added to an equal weight of ethanol (96% v/v, derived from cane sugar) at pH 6 for about 2 to 3 weeks at room temperature. The mixture was pressed using a wine grape press, then filtered to remove any solid material. The filtrate was retained.

Non-ethoxylated green tea extract was prepared by the addition of green tea leaves to ethanol (96% v/v, derived from cane sugar) at pH 6 in a 1:4 ratio. The resultant slurry was allowed to stand at room temperature for 1 hour. The mixture was filtered to remove any solid material, and the filtrate was retained.

The filtrates were combined in the proportions as shown in Table 12.

TABLE 12

| Component | Proportion (%, v/v) |
|---|---|
| EVC 1:4 extract | 9.7 |
| Wine grape extract | 9.7 |
| Non-ethoxylated turmeric extract | 7.5 |
| Non-ethoxylated ginger extract | 25.9 |
| Non-ethoxylated citrus pulp extract | 37.5 |
| Non-ethoxylated green tea extract | 9.7 |

The resultant blend was reduced to 25% of the original volume using vacuum distillation to provide a de-alcoholized blend. The de-alcoholized blend was then combined with concentrates in the proportions shown in Table 13.

TABLE 13

| Concentrate | Proportion (%, v/v) |
|---|---|
| De-alcoholized blend (Table 12) | 15.8 |
| Purple carrot concentrate (60 Brix) | 68.4 |
| Blueberry concentrate (65 Brix) | 15.8 |

The mixture according to Table 13 was placed into a stainless steel vat and blended for 2 hours. The blended mixture was then combined with dry products according to Table 14 and blended for an additional 1 hour.

TABLE 14

| Component | Quantity | |
|---|---|---|
| Extract blend (as in Table 13) | 100 | mL |
| Purple carrot powder | 8.4 | g |
| Ascorbic acid | 1 | g |
| Potassium sorbate | 0.05 | g |
| Sodium metabisulphite | 0.06 | g |

The cell cultures and testing were as described in Example 39.

For the serum withdrawal experiments, LNCaP prostate cancer cells were deprived of serum for 7 days and 0.5 million cells were re-plated to T75 flasks in the presence of serum. The cells were cultured in the absence or presence of EVC-PCBC at varying concentrations for two weeks.

For the contact inhibition experiments, PC-3 cells were confluent for three days and replated to new culture flasks at low density. The Cells were then treated with different doses of EVC-PCBC for three days prior to analysis of cell cycle status (with re-entry to the cell cycle indicative of cancer re-occurrence).

Results

EVC-PCBC was tested on non-synchronised LNCaP, MDA-PCa-2b and PC-3 prostate cancer cell lines and MDA-MB-231 breast cell lines.

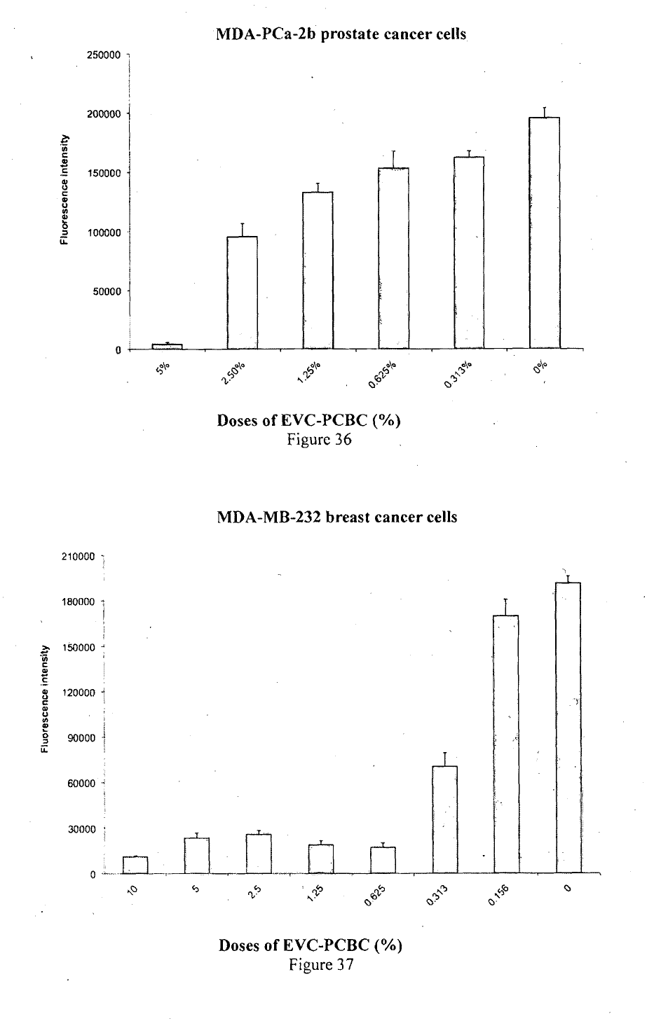

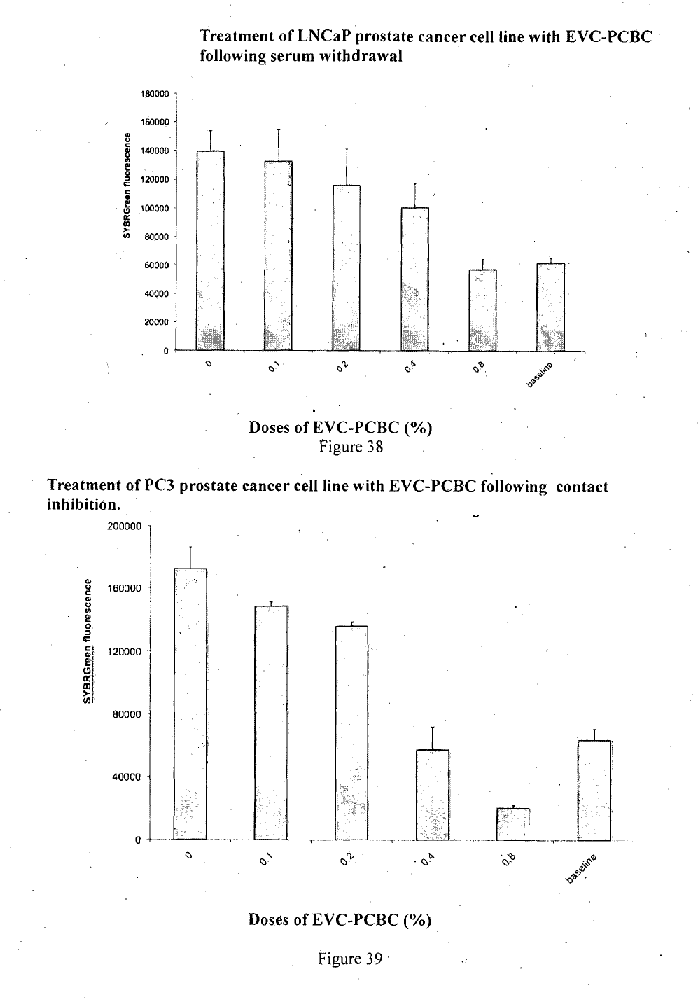

The resultant data are depicted graphically in FIGS. 35 to 39.

Data for the treatment of quiescent PC-3 prostate cancer cells with EVC-PCBC is also presented in Table 15.

TABLE 15

| Sample | G0-G1 | S | G2-M |
|---|---|---|---|
| Control 1 | 64.6 | 12.6 | 22.8 |
| Control 2 | 64.5 | 11.8 | 23.6 |
| Control 3 | 64.9 | 11.9 | 23.1 |
| Average | 64.7 | 12.1 | 23.2 |
| 1: 0.2% EVC-PCBC | 71.4 | 7.34 | 21.1 |
| 2: 0.2% EVC-PCBC | 70.1 | 8.12 | 21.7 |
| 3: 0.2% EVC-PCBC | 70.3 | 8.03 | 21.5 |
| Average | 70.6 | 7.8 | 21.4 |
| 4: 0.4% EVC-PCBC | 78.3 | 4.85 | 16.8 |
| 5: 0.4% EVC-PCBC | 78.8 | 4.7 | 16.4 |
| 6: 0.4% EVC-PCBC | 78 | 4.9 | 17.1 |
| Average | 78.4 | 4.8 | 16.8 |

From the results presented in Table 15 and FIG. 39, it can be seen that EVC-PCBC increased the proportion of cancer cells at G0-G1 phase, but decreased the proportion in S and G2-M phase, suggesting that EVC-PCBC is likely to inhibit PC-3 quiescent cancer cells from re-entering the cell cycle, thereby inhibiting cancer reoccurrence.

Human Testimonials

The results from a human volunteer who incorporated the EVC-PCBC into his diet are detailed below.

M, 44, 240 mL of EVC-PCBC per day (3×80 mL mixed with equal volume water) for 26 weeks. Skin cancer with metastasis in lung and liver. 240 mL of EVC-PCBC per day translates to the ethoxylated extract derived from 3.75 g dry oak per day. First diagnosed with skin cancer at the back of the leg 4 years ago. Successful tumour removal by surgery. Subsequent PET scans confirmed lung metastasis (3 cm diameter) and liver metastasis (3 cm diameter). Doctors estimate with treatment (chemotherapy) has only 6 months life expectancy. Chose not to follow the chemotherapy route, given the poor prognosis. After 1 month of very high dose EVC-PCBC consumption, PET scan reveals that lung and liver tumours are reduced to half size. After 2 months EVC-PCBC consumption, PET scan unable to find the tumours. Subsequent scans and confirmation from skin cancer specialist over the next 3 years confirm that is cancer free. Stopped taking the EVC-PCBC after 6 months on consumption. It should also be noted that did take other supplements during this timeframe and expanded the spiritual dimensions of his life (Mary MacKillop church in Brisbane).

### Example 36

### Low Dose EVC/Ethoxylated Food Extract is Unpleasant Tastewise, Well Tolerated and Produces Significant Health Outcomes in Cancer

This example describes the preparation of ethoxylated oak (ethoxylated vescalagin-rich/castalagin-rich) extract (EVC) formulated into a mixture of ethoxylated food extracts and fresh ginger juice. This mixture is given the name EVC food extract. The mixture is an example of a dietary supplement or an additive package for foods.

Materials and Methods

EVC Food Extract

Grape brandy (4 kg, 62% v/v) with pH 3.5 was added to European oak chips (1 kg, Quercus Robur/Quercus petraea L.) and allowed to stand for a period of 2 months at room temperature. The mixture was then filtered to remove the solid material and the filtrate, being an alcoholic EVC extract, was retained.

Ethoxylated turmeric and ginger extracts were prepared by placing whole fruit in a juice extractor. The resultant pulp/skin was added to an equal weight of grape brandy (62% alcohol) at pH 3.5 for about 4 weeks at room temperature. The mixture was filtered to remove any solid material, and the filtrate was retained.

Ethoxylated extracts of olive leaf were prepared by the addition of fresh olive leaf to ethanol (96% v/v, derived from grapes) at pH 4.3 in a 1:4 ratio. Typically, the mixture was allowed to sit for about one week prior to filtering, to allow for complete extraction and ethyoxylation.

Ethoxylated citrus skin extracts were prepared individually from whole fruit (orange, lemon, mandarin, Tahitian lime and kaffir lime) placed in a juice extractor. The resultant pulp/skin was added to an equal weight of ethanol (96% v/v, derived from grapes) at pH 4.3 for about 3 weeks at room temperature. The mixture was pressed using a wine grape press, then filtered to remove any solid material. The filtrate was retained.

Ethoxylated green tea extract was prepared by the addition of green tea leaves to ethanol (96% v/v, derived from grapes) at pH 4.3 in a 1:4 ratio. The resultant solution was allowed to stand at room temperature for 1 hour. The mixture was filtered to remove any solid material, and the filtrate was retained.

Ethoxylated tarragon extract was prepared by drying freshly picked tarragon (Tagetes lucida) leaves and adding to ethanol (96% v/v, derived from grapes, pH 4.3) in a 1:4 ratio. The resultant mixture was allowed to stand at room temperature for 5 to 7 days. The mixture was then filtered to remove any solid material, and the filtrate was retained.

The filtrates were combined in the proportions as shown in Table 16.

TABLE 16

| Component | Proportion (% w/w) |
|---|---|
| Ethoxylated EVC extract | 6.6 |
| Ethoxylated turmeric extract | 6.5 |
| Ethoxylated ginger extract | 32.6 |
| Ethoxylated olive leaf extract | 5.2 |
| Ethoxylated citrus skin extracts | 26 |
| Ethoxylated tarragon extract | 6.6 |
| Ethoxylated green tea extract | 6.6 |
| Ethoxylated grape seed/skin extract | 6.6 |
| Raspberry juice concentrate (62 Brix) | 3.3 |

The resultant blend of filtrates was combined with an equal volume of fresh ginger juice. The fresh ginger juice was obtained by placing whole ginger into a juice extractor to produce ginger juice and ginger pulp. The combined filtrate blend and ginger juice was further fortified with ascorbic acid, citric acid and hesperidin, as shown in Table 17.

TABLE 17

| Component | Quantity |
|---|---|
| Filtrate blend (as in Table 16)/ginger juice | 100 mL (50 mL filtrate |
| combination | blend from Table |
| | 16/50 mL ginger juice) |
| Ascorbic acid | 0.11 g |
| Citric acid | 0.15 g |
| Hesperidin | 0.095 g |

The final product is named EVC food extract.

Cell Culture and Preparation for Testing Efficacy of EVC Food Extract

Exponentially growing cells were cultured in six-well plates, T25 flasks or T75 flasks for 3 days before treatment with EVC food extract. EVC food extract was first diluted to 0.8% in each culture medium and sterilised by filtration. Further dilutions were made in the corresponding medium to achieve lower concentrations at 0.2 and 0.4%. All cell lines were treated for 72 h and the untreated cells served as controls.

Results

The EVC food extract was tested on the J82 bladder cancer cell line, the HT 1376 bladder cancer cell line, the AGS stomach cancer cell line, the breast cancer cell lines MDA-MB-468 and MDA-MB-231, the colon cancer cell lines LS180 and DLD-1, the PC-3 and LNCaP prostate cancer cell lines and the androgen insensitive LNCaP (AI-LNCaP) prostate cancer cell line.

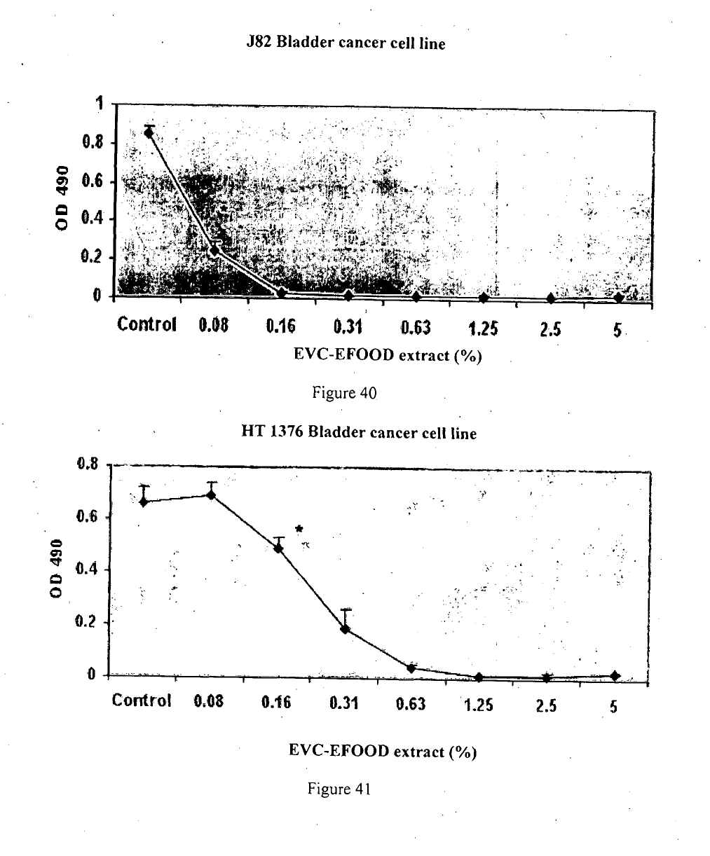

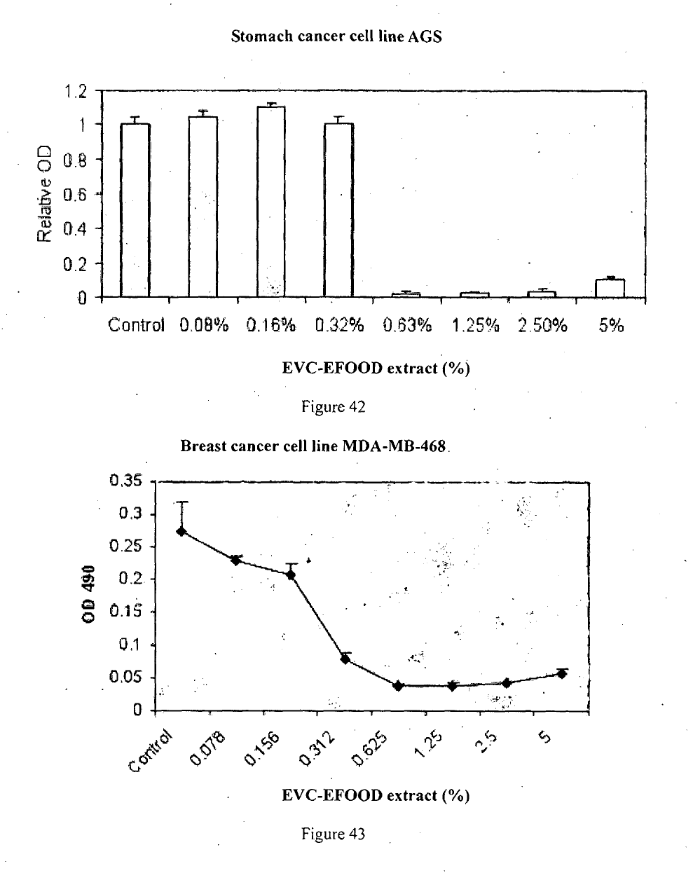

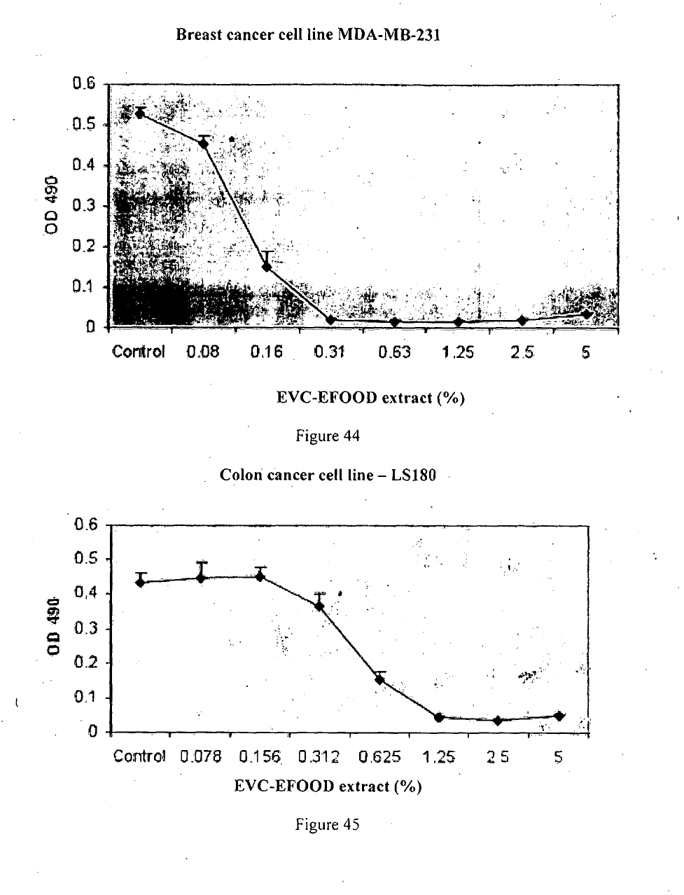

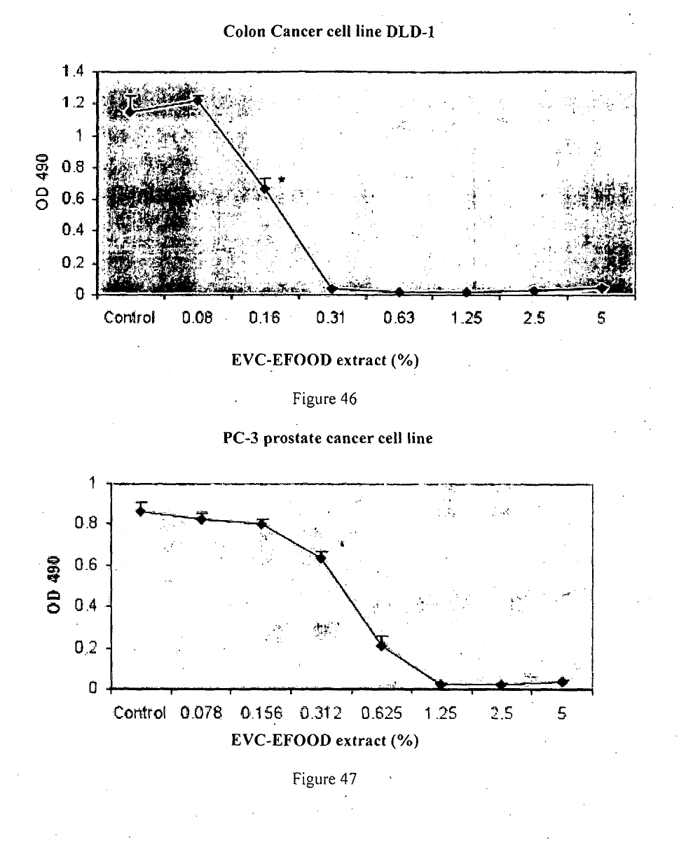

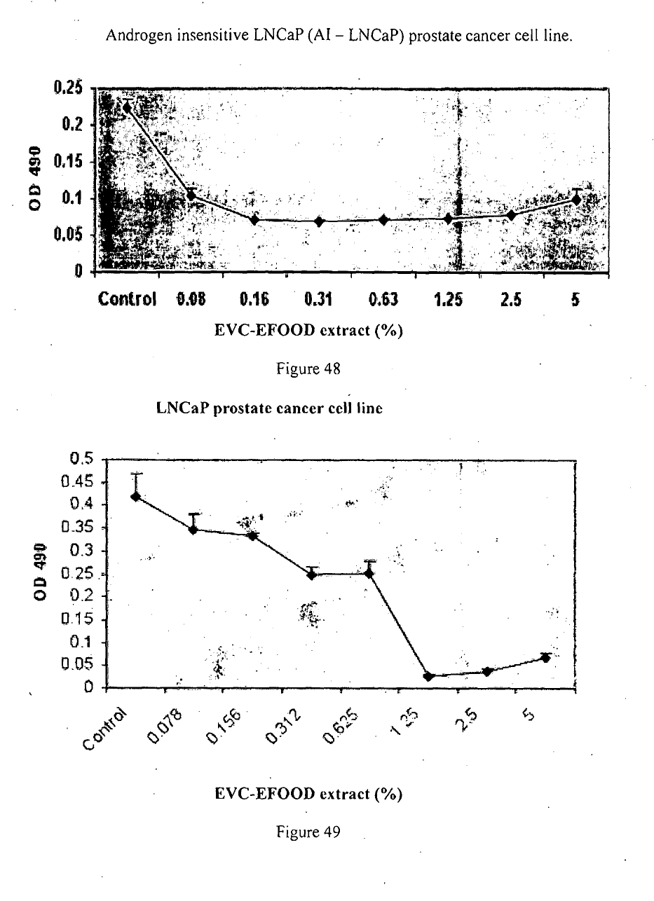

The resultant data are depicted graphically in FIGS. 40 to 49.

### Example 37

### Low Dose EVC/Ethoxylated Food Extract/Non-Ethoxylated Green Tea Formulation Produces Significant Health Outcomes in Cancer

This example describes the preparation of a green tea fortified with polyphenol-rich extracts (EVC-EFOOD TEA). The EVC-EFOOD TEA was tested on a Pane 5.04 pancreatic cancer cell line and LNCaP, PC3 and DU145 prostate cancer cell lines.

Materials and Methods

EVC-EFOOD TEA is a blend of four components:

(a) ethoxylated oak-aged winegrape extract (ethoxylated vascalagin/castalagin rich extract (EVC) combined with ethoxylated grape seed/skin extract);
(b) ethoxylated food extracts (ginger, turmeric, citurs skins, olive leaf, green tea extract);
(c) Baikal skullcap (Scutellaria baicalensis); and
(d) Panax ginseng;
dried on a loose-leaf green tea substrate.

The preparation of the ethoxylated oak-aged winegrape extract and the ethoxylated food extracts is described in Example 36.

EVC-EFOOD TEA was prepared as follows.

Dried root of Scutellaria baicalensis (100 g) was soaked with ethanol (95%, pH 6.0) for 4 weeks, filtered and filtrate retained.

Panax ginseng powder (100 g, Jian Pharmaceuticals, PRC) was soaked with ethanol (95%, pH 6.0) for 4 weeks, filtered and filtrate retained.

The extracts were combined in the proportions as shown in Table 18.

TABLE 18

| Concentrate | Proportion |
|---|---|
| Extract mixture described in Example 36, | 95% |
| Table 16 | |
| Baikal skullcap extract | 3% |
| Panax ginseng | 2% |

The resultant alcoholic mixture described in Table 16 (1 kg) was mixed into loose leaf gunpowder green tea (4 kg, Madura Tea Estate, NSW, Australia) and allowed to dry at ambient conditions for 2 days.

EVC-EFOOD TEA (2 g) was added to boiling water (150 mL) and allowed to extract for 5 minutes. A 10% solution was prepared from the extract and used as a stock solution. From the stock solution (10%), further serial dilutions were made to produce solutions ranging from 5% down to 0.08%.

The cell cultures and testing were as described in Example 39. Specifically, measurement of DNA content was undertaken using SYBR green in a surrogate assay for cell numbers.

Results

The EVC-EFOOD TEA was tested on a Panc 5.04 pancreatic cancer cell line and LNCaP, PC3 and DU145 prostate cancer cell lines.

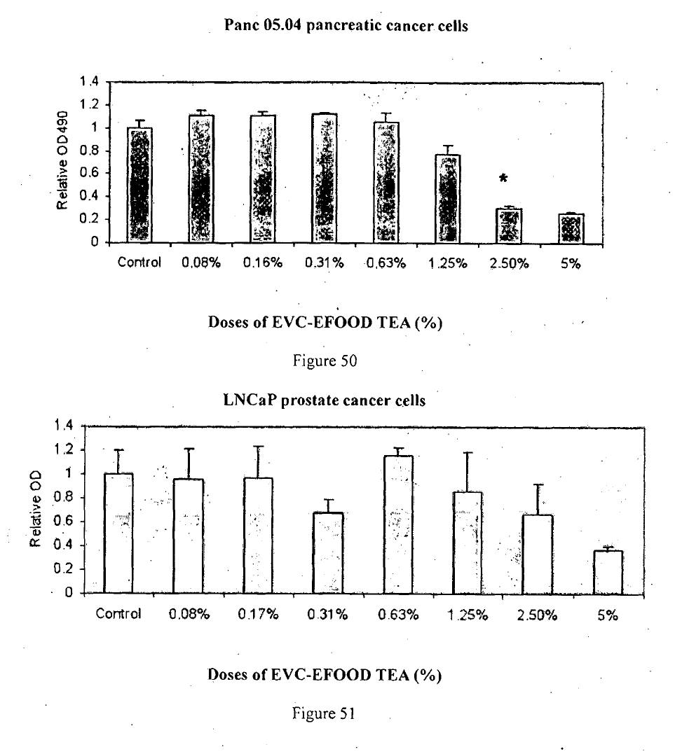

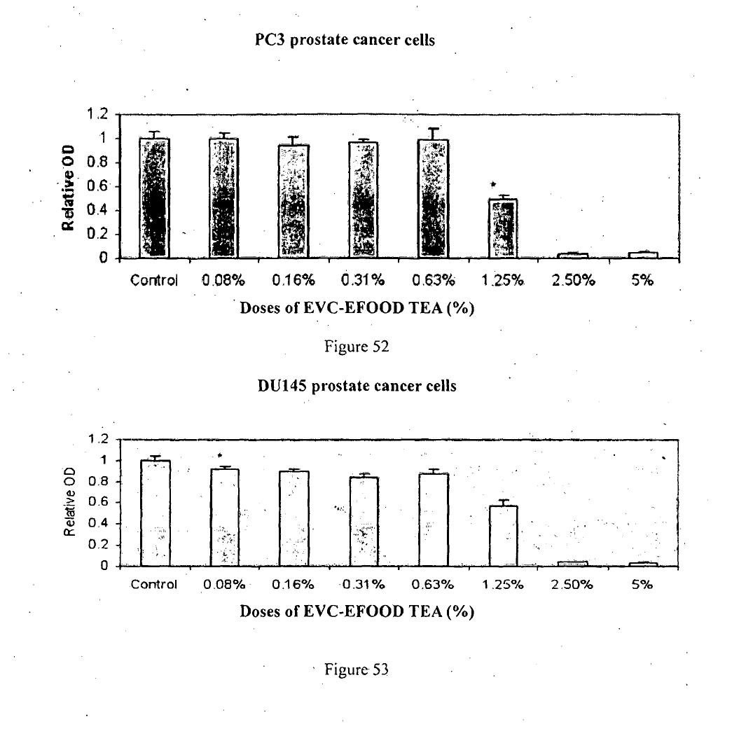

The resultant data are depicted graphically in FIGS. 50 to 53.

Best results in cancer cell number reduction were achieved with the DU145 cell line (moderate metastatic potential, not hormone sensitive) and PC3 cell line (high metastatic potential, not hormone sensitive).

### Example 38

### Low Dose EVC/Ethoxylated Food Extract (Example 36 Above) Cancer Results are Improved by the Addition of Anthocyanin Rich Fruit Juice Concentrates to the Formulation

This example describes the preparation of fully ethoxylated extract EVC food extract blended into a coloured substrate (CS) base. The rationale for the testing of this particular food formulation was to assess the value of highly coloured substrates replacing fresh ginger juice in the same prostate cancer cell lines.

Materials and Methods

The fully ethoxylated extract formula described in Example 36 (Table 16) was added to fruit juice concentrates in the proportions as shown in Table 19.

TABLE 19

| Concentrate | Proportion (% v/v) |
|---|---|
| Filtrate blend (Table 16) | 20 |
| Blueberry concentrate (65 Brix) | 8 |
| Red grape concentrate (68 Brix) | 48 |
| White grape concentrate (68 Brix) | 19 |
| Elderberry concentrate | 5 |

The cell cultures and testing were as described in Example 39.

Results

The EVC food extract and EVC food extract/CS were tested on the LNCaP and PC3 prostate cancer cell lines.

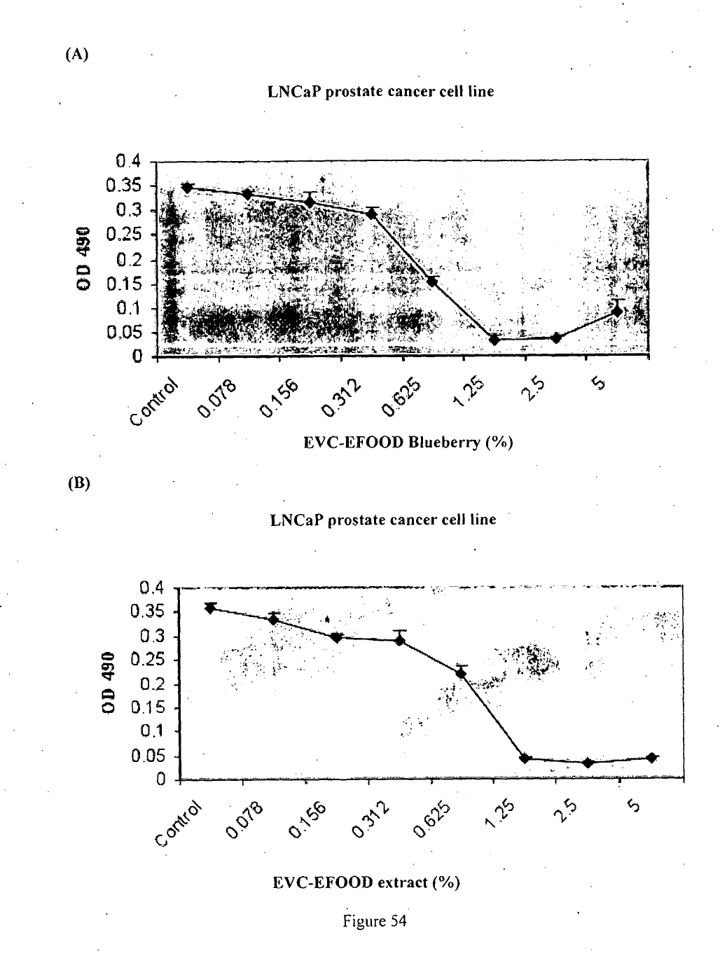

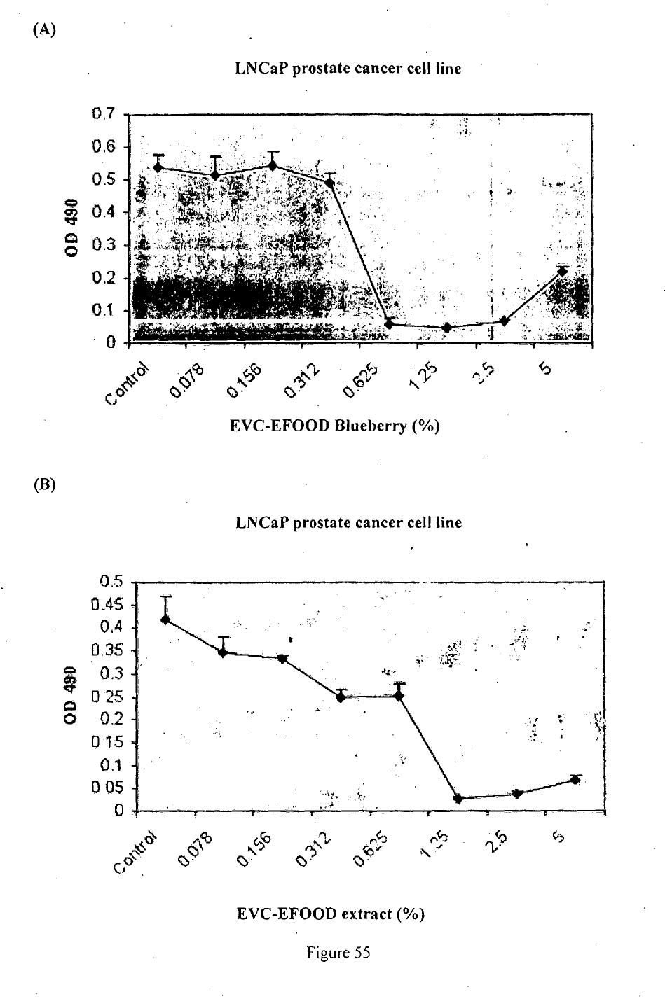

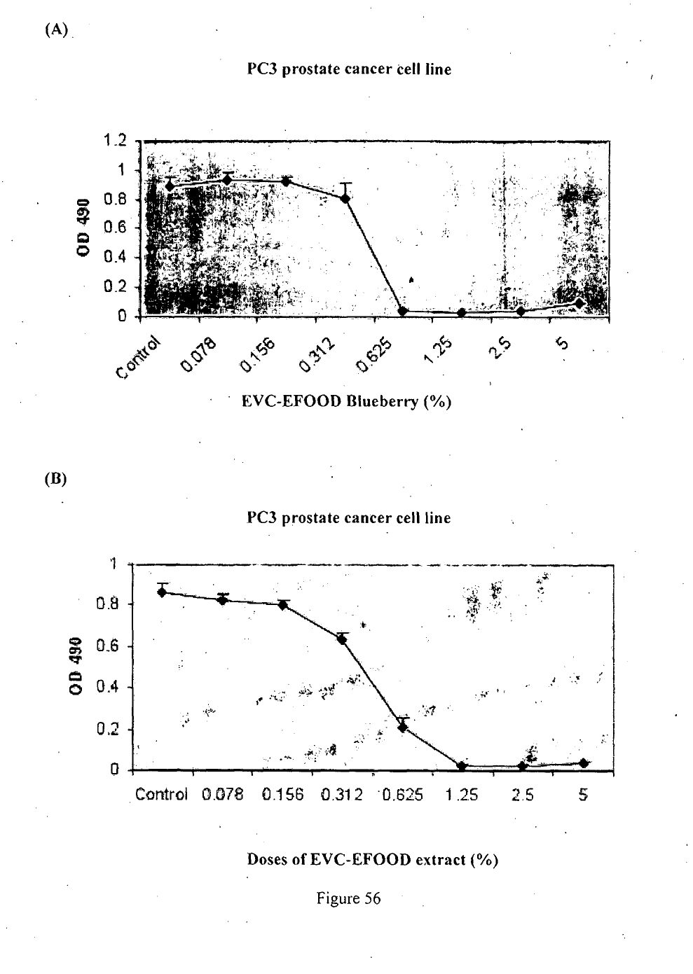

The resultant data are depicted graphically in FIGS. 54 to 56.

### Example 39

### Low Dose EVC/Non-Ethoxylated Food Extracts/Ethoxylated Food Extracts/Anthocyanin Rich Non-Ethoxylated Fruit Juice Concentrates with Significant Cancer Results

This example describes the preparation of ethoxylated oak (ethoxylated vescalagin-rich/castalagin-rich) extract (EVC), its formulation into a mixture of ethoxylated extracts and non-ethoxylated extracts, and highly coloured fruit juice concentrate to form a concentrated beverage formula for use in the prevention and treatment of cancer. This food composition is given the name EVC blueberry juice blend.

Materials and Methods

Specifically, the EVC is formulated with ethoxylated extracts (ginger, turmeric and grape seed/skin extracts), and non-ethoxylated extracts (citrus skin extracts, olive leave extract, green tea extract, tarragon) on a coloured substrate (CS) base of fruit juice concentrates (blueberry, raspberry, elderberry).

Grape brandy (4 kg, 62% v/v) with pH 3.5 was added to European oak chips (1 Kg, Quercus Robur/Quercus petraea L.) and allowed to stand for a period of 2 months at room temperature. The mixture was then filtered to remove the solid material and the filtrate, being an ethanolic EVC 1:4 extract, was retained.

An extract of red wine grape skins and seeds, which is a byproduct of a winemaking process, and therefore comprises ethoxylated components was added to ethanol (96% v/v, derived from cane sugar) at pH 6 in a 1:1 ratio. The resultant solution had a pH of 4.5 and was allowed to stand at room temperature for 2 to 3 weeks. The mixture was filtered to remove any solid material, and the filtrate was retained.

Ethoxylated turmeric and ginger extracts were prepared by placing whole fruit in a juice extractor. The resultant pulp/skin was added to an equal weight of grape brandy (62% alcohol) at pH 3.5 for about 3 weeks at room temperature. The mixture was filtered to remove any solid material, and the filtrate was retained.

Non-ethoxylated extracts of olive leaf were prepared by the addition of fresh olive leaf to ethanol (96% v/v, derived from cane sugar) at pH 6 in a 1:4 ratio. Typically, the mixture was left for one week prior to filtering, to allow for complete extraction.

Non-ethoxylated citrus skin extracts were prepared individually from whole fruit (orange, lemon, mandarin, Tahitian lime and kaffir lime) placed in a juice extractor. The resultant pulp/skin was added to an equal weight of ethanol (96% v/v, derived from cane sugar) at pH 6 for about 2 to 3 weeks at room temperature. The mixture was pressed using a wine grape press, then filtered to remove any solid material. The filtrate was retained.

Non-ethoxylated green tea extract was prepared by the addition of green tea leaves to ethanol (96% v/v, derived from cane sugar) at pH 6 in a 1:4 ratio. The resultant solution was allowed to stand at room temperature for 1 hour. The mixture was filtered to remove any solid material, and the filtrate was retained.

The filtrates were combined in the proportions as shown in Table 20.

TABLE 20

| Component | Proportion (%, w/w) |
|---|---|
| EVC 1:4 extract | 3.6 |
| Ethoxylated turmeric extract | 6.6 |
| Ethoxylated ginger extract | 32 |
| Ethoxylated grape seed/grape skin extract | 6.4 |
| Non-ethoxylated olive leaf extract | 5 |
| Non-ethoxylated citrus skin extracts | 25 |
| Non-ethoxylated green tea extract | 6.4 |
| Non-ethoxylated tarragon extract | 3.2 |
| Hesperidin | 5 |
| Ascorbic acid | 6 |
| Citric acid | 0.8 |

The resultant blend was reduced to 25% of the original volume using vacuum distillation to provide a de-alcoholized extract blend. The de-alcoholized extract blend was then combined with concentrates in the proportions shown in Table 21.

TABLE 21

| Concentrate | Proportion (%, v/v) |
|---|---|
| De-alcoholized blend | 20 |
| Blueberry concentrate (65 Brix) | 7.3 |
| Raspberry concentrate (62 Brix) | 3.4 |
| Elderberry concentrate (60 Brix) | 4.2 |
| Shiraz grape concentrate (68 Brix) | 46.3 |
| White grape concentrate (68 Brix) | 18.8 |

The resultant mix comprised 95% blueberry/grape concentrates and 5% de-alcoholised blend (EVC blueberry juice blend).

Cell Culture and Preparation for Testing Efficacy of EVC Blueberry Juice Blend

Lymph node-metastasised prostate cancer cells (LNCaP; CRL-1740, ATCC) and bone metastasised androgen receptor negative prostate cells (PC-3; CRL-1435; American Type Culture Collection, Manassas, Va., USA) were grown in Roswell Park Memorial. Institute (RPMI) 1640 media supplemented with 10% fetal calf serum (Hyclone), penicillin (100 units/mL) and streptomycin (100 mg/mL).

Bone metastasised androgen receptor positive prostate cells (MDA-PCa-2b; CRL-2422, ATCC) were grown in F-12K nutrient mixture (Invitrogen) supplemented with 20% fetal calf serum, penicillin (100 units/mL), streptomycin (100 mg/mL), epidermal growth factor (10 ng/mL), 5 mM-phos-phoethanolamine, cholera toxin (25 ng/mL), hydrocortisone (0.1 ng/mL), selenious acid (45 nM) and insulin (5 mg/mL).

All the cell lines were cultured at 37.8° C. in an incubator providing a humidified environment in the presence of 5% CO₂ and 95% air.

Treatment Protocol for Testing Efficacy of EVC Blueberry Juice Blend

Exponentially growing cells were cultured in six-well plates, T25 flasks or T75 flasks for 3 days before treatment with EVC blueberry juice blend. EVC blueberry juice blend was first diluted to 0.8% in each culture medium and sterilised by filtration. Further dilutions were made in the corresponding medium to achieve lower concentrations at 0.2 and 0.4%. All cell lines were treated for 72 h and the untreated cells served as controls.

Detection of Cellular DNA Synthesis Following EVC Blueberry Juice Blend Treatment

The cells were treated with EVC blueberry juice blend in T25 flasks and their DNA synthesis was monitored by the incorporation of 5-ethynyl-20-deoxyuridine (EdU) to newly synthesized DNA with Click-iTe EdU Flow Cytometry Assay Kit (C35002; Invitrogen). Following this, 8 hours before completion of treatment, the exhausted medium was removed and the cells were incubated in the fresh medium containing 10 mM-EdU and EVC blueberry juice blend at each corresponding concentration.

The cells were then trypsinised and fixed in cold 70% ethanol in PBS at 48° C. overnight. The fixed cells were then washed with PBS containing 5% fetal calf serum and the incorporated EdU was labelled with reaction cocktails according to the assay instructions. The labelled cells were analysed by a flow cytometer (BD FACSCalibur; BD Biosciences) equipped with CellQuest Pro software (BD Biosciences) to determine the percentage of cells with incorporated EdU.

Cell Cycle Analysis of Treated Cells with Addition of EVC Blueberry Juice Blend

The cell cycle distribution of the treated cells was determined by flow cytometry. The cells treated in T25 flasks were collected and fixed in cold 70% ethanol in PBS overnight at 48° C. After being washed in PBS, the cells were incubated in PBS containing 100 mg/mL RNase and 20 mg/mL propidium iodide at 37.8° C. for 60 min. Thereafter, the DNA content was analysed by the flow cytometer.

Immunocytochemistry Determination

Expression of Ki-67 and phospho-retinoblastoma (pRb) protein at Ser807/811 was determined by immunostaining. The treated cells in T75 flasks were trypsinised, fixed in 10% buffered formalin solution overnight at 48° C., and then processed for paraffin blocks. Sections with 5 mm thickness were baked at 60.8° C. for 1 hour, deparaffinised in xylene, re-hydrated in graded ethanol and distilled water, and subjected to antigen retrieval in Tris-EDTA solution.

The sections were then blocked with 10% horse serum and incubated with each primary antibody for 20 h at 48° C. The sections were washed with Tris-buffered saline with Tween (TBST) and sequentially labelled with a biotinylated secondary antibody (RA-1000) and Vectastain ABC kit (PK-4000) from Vector Laboratories.

The immuno-labelling of indicated antigens was detected with 3,3′-diaminobenzidine (K3468; Dako). The immuno-labelled sections were counterstained with haematoxylin and cover-slipped. The primary antibodies to Ki-67 (RM-9106-S) and pRb at Ser807/811 (9308) were purchased from Labvision and Cell Signalling Technology, respectively.

Quantification of Immunostained Cells

Immunostained sections were scanned by an automated cellular imaging system equipped with automated cellular imaging system (ACIS) software (ACIS III; Dako) to acquire digital images. Next, two colour thresholds were selected on the images to distinguish between the positively stained (dark brown) and negatively stained (blue) cells. The cells in ten randomly selected fields were then analysed. The percentage of positive cells of each sample was calculated using the following formula:

positive cells/(positive cells+negative cells)×100%

Immunoblotting

The cells treated in six-well plates were harvested in lysis buffer (50 mM-Tris-pH 8, 150 mM-NaCl, 1% Igepal CA-630, 0.5% sodium deoxycholate, 0.1% SDS) supplemented with protease inhibitor cocktail (11836145001; Roche) and 50 mM sodium fluoride.

Protein concentration was quantified using a Bio-Rad Protein Assay (Bio-Rad). The proteins in the lysates were separated on SDS-PAGE and transferred onto a nitro-cellulose membrane (RPN303E; Amersham Biosciences).

The membranes were blocked with 1% non-fat milk in phosphate-buffered saline-Tween (PBST) for 30 minutes and incubated with primary antibody for 1-2 days at 48° C. The blots were washed, and incubated with appropriate secondary antibodies conjugated with peroxidase (Sigma-Aldrich) and Precision Strep Tactin-Peroxidase Conjugate (161-0380; Bio-Rad) for 3 hours. The immuno-labelling was revealed by SuperSignal West Pico Chemiluminescent Substrate (34 078; Thermo Scientific).

Immuno-labelled protein bands were captured by a charge-coupled device (CCD) camera built in a gel documentation and analysis system (Syngene). The band intensity on saved digital images was measured using Phoretics 1D Advanced software (Non Linear Dynamics) and the ratio of band intensity between proteins of interest and loading control was calculated. The primary antibodies against Cyclin E (Sc-198), cyclin-dependent kinase (CDK) 2 (Sc-748), CDK4 (Sc-749), cell division cycle (CDC) 6 (Sc-9964) and minichromosome maintenance complex (MCM7) (Sc-22782) were obtained from Santa Cruz Biotechnology; antibodies against Cyclin D1 (C7464) were obtained from Sigma-Aldrich; the antibodies to α-tubulin (ab7291) and glyceraldehyde 3-phosphate dehydrogenase (ab8245) were obtained from Abcam.

Statistical Analysis

Statistical comparison was performed using NCSS PASS version 12.0 (NCSS Statistical and Power Analysis Software). The processed data were analysed using one-way ANOVA to determine if a significant change had occurred. Fisher's least significant difference multiple-comparison test (significance: P<0.05) and Kruskal-Wallis multiple-comparison z-value test (significance: z-value of 1.96) were applied to determine which doses of EVC blueberry juice blend had a significant effect.

Results

To determine the effect of EVC blueberry juice blend treatment on cell cycle phase distribution, LNCaP, PC-3 and MDA-PCa-2b cells were treated with EVC blueberry juice blend (up to 0.8% in corresponding culture medium) for 72 h and then stained with propidium iodide and analysed by flow cytometry.

Treatment with 0.2-0.4% EVC blueberry juice blend blocked cell cycle progression in LNCaP, PC-3 and MDA-PCa-2b cell lines as manifested by an accumulation of cells in the G0/G1 phase and a reduction in the percentage of cells in the S or G2/M phase as detailed in Table 22. Specifically, Table 22 details the cell cycle phase distribution of lymph node-metastasised prostate cancer cells (LNCaP), bone metastasised androgen receptor negative prostate cells (PC-3) and bone metastasised androgen receptor positive prostate cells (MDA-PCa-2b) measured by flow cytometry (average percentage of cells with standard deviation in three independent experiments).

TABLE 22

| | G0/G1 | | | | G2/M | |
|---|---|---|---|---|---|---|
| | phase | | S phase | | phase | |
| | Mean | SD | Mean | SD | Mean | SD |
| | LNCaP | | | | | |
| 0% EVC Blueberry | 73.1^{a} | 1.0 | 6.3^{a} | 0.3 | 20.0^{a} | 0.8 |
| 0.2% EVC Blueberry | 73.7^{a} | 0.8 | 6.2^{a} | 0.2 | 19.5^{a} | 0.8 |
| 0.4% EVC Blueberry | 79.6^{b} | 0.9 | 4.9^{b} | 0.3 | 15.5^{b} | 0.7 |
| 0.8% EVC Blueberry | 82.3^{c} | 0.8 | 4.5^{b} | 0.6 | 13.0^{c} | 0.7 |
| | PC-3 | | | | | |
| 0% EVC Blueberry | 60.6^{a} | 0.7 | 16.0^{a} | 0.3 | 23.3^{a} | 0.3 |
| 0.2% EVC Blueberry | 62.0^{b} | 0.2 | 15.6^{a} | 0.2 | 22.3^{a} | 0.1 |
| 0.4% EVC Blueberry | 66.7^{c} | 1.2 | 12.7^{b} | 0.8 | 21.0^{b} | 1.0 |
| 0.8% EVC Blueberry | 70.3^{d} | 0.7 | 8.6^{c} | 0.1 | 20.5^{b} | 0.5 |
| | MDA-PCa-2b | | | | | |
| 0% EVC Blueberry | 73.2^{a} | 0.6 | 11.4^{a} | 0.4 | 11.5^{a} | 0.6 |
| 0.2% EVC Blueberry | 74.8^{b} | 0.4 | 11.0^{a} | 0.1 | 10.8^{a} | 0.4 |
| 0.4% EVC Blueberry | 76.7^{c} | 0.3 | 10.3^{b} | 0.1 | 9.5^{b} | 0.4 |
| 0.8% EVC Blueberry | 82.5^{d} | 0.1 | 7.0^{c} | 0.2 | 6.3^{c} | 0.2 |

^{a,b,c,d}Mean values within a column with unlike superscript letters were significantly different (P < 0.05)

A further retardation of cell cycle progression was noted with 0.8% EVC blueberry juice blend treatment.

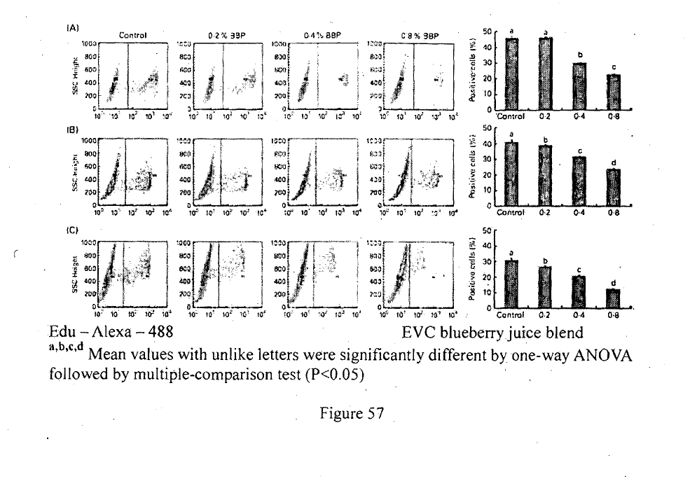

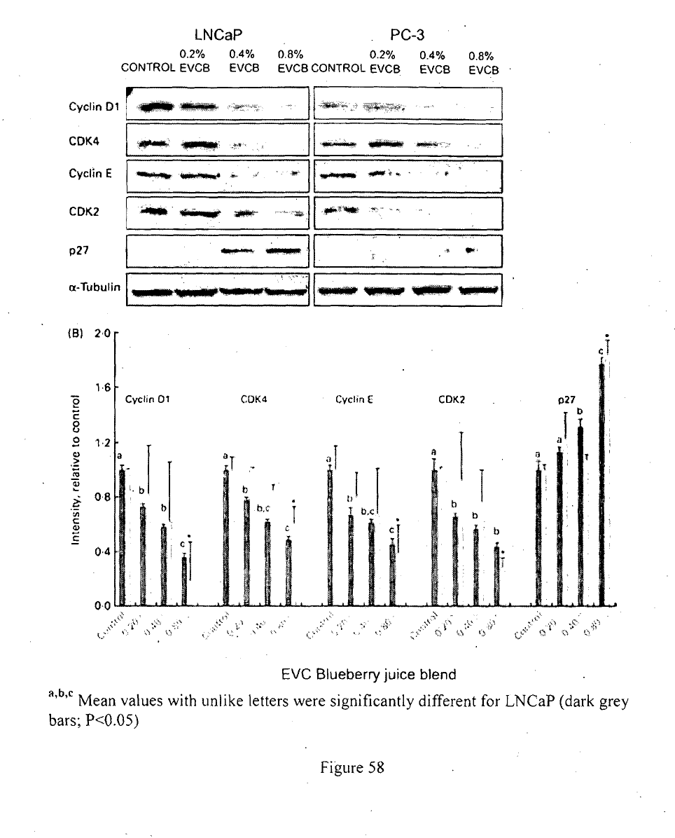

To confirm the reduction of cells in the S phase, DNA synthesis was monitored by EdU incorporation using flow cytometry. Treatment with EVC blueberry juice blend at the concentration of 0.2-0.4% decreased DNA synthesis, as shown in FIG. 57.

Specifically, FIG. 57 (A) shows the results for lymph node-metastasised prostate cancer cells, LNCap, FIG. 57 (B) shows the results for bone metastasised androgen receptor negative prostate cells, PC-3, and FIG. 57 (C) shows the results for bone metastasised androgen receptor positive prostate cells, MDA-PCa-2b following incubation with EVC blueberry juice blend for 72 h, labelled with 10 mM 5-ethynyl-20-deoxyuridine for 8 h, and then analysed by flow cytometry. The values are means, with standard deviations represented by vertical bars.

The proliferation of lymph node-metastasised prostate cancer cells (LNCaP), bone metastasised androgen receptor negative prostate cells (PC-3) and bone metastasised androgen receptor positive prostate cells (MDA-PCa-2b) cells measured by Ki-67 staining is detailed in Table 23.

TABLE 23

| | | | | | MDA- | |
|---|---|---|---|---|---|---|
| | LNCaP | | PC-3 | | PCa-2b | |
| | Mean | SD | Mean | SD | Mean | SD |
| 0% EVC Blueberry | 74.9^{a} | 2.9 | 76.8^{a} | 7.3 | 45.7^{a} | 4.8 |
| 0.2% EVC Blueberry | 75.9^{a} | 3.0 | 71.8^{a,b} | 2.1 | 35.8^{a} | 2.5 |
| 0.4% EVC Blueberry | 59.2^{b} | 3.5 | 63.9^{b} | 0.1 | 32.5^{b} | 1.8 |
| 0.8% EVC Blueberry | 24.8^{c} | 9.7 | 24.8^{c} | 2.0 | 10.2^{c} | 1.5 |

^{a,b,c}Mean values, within a column with unlike superscript letters were significantly different (P < 0.05)

The data shown in Table 23 is the average percentage of Ki-67 positive cells with standard deviation from ten randomly selected fields in three independent experiments.

Data relating to the phosphorylation of retinoblastoma protein in lymph node metastasised prostate cancer cells (LNCaP), bone metastasised androgen receptor negative prostate cells (PC-3) and bone metastasised androgen receptor positive prostate cells (MDA-PCa-2b) cells is shown in Table 24.

TABLE 24

| | | | | | MDA- | |
|---|---|---|---|---|---|---|
| | LNCaP | | PC-3 | | PCa-2b | |
| | Mean | SD | Mean | SD | Mean | SD |
| 0% EVC Blueberry | 27.8^{a} | 4.9 | 66.5^{a} | 1.7 | 20.7^{a} | 2.8 |
| 0.2% EVC Blueberry | 18.4^{b} | 1.7 | 27.6^{b} | 1.1 | 7.2^{b} | 1.4 |
| 0.4% EVC Blueberry | 7.9^{c} | 1.5 | 11.4^{c} | 0.3 | 6.7^{b} | 2.1 |
| 0.8% EVC Blueberry | 5.9^{c} | 1 | 7.0^{d} | 0.0 | 3.8^{c} | 1.5 |

^{a,b,c,d}Mean values within a column with unlike superscript letters were significantly different (P < 0.05)

The data shown in Table 24 is the average percentage of phospho-retinoblastoma protein-positive cells with standard deviation from ten randomly selected fields in three independent experiments.

These results suggest that EVC blueberry juice blend blocks a transition from the G1 to the S phase in treated cells. Experiments were then undertaken to determine the levels of cycling CDK and the inhibitor p27 involved at the G1-S transition in LNCaP and PC-3 cells. As can be seen from FIG. 58, EVC blueberry juice blend treatment decreased the protein levels of cyclin D1/CDK4 and cyclin E/CDK2 in both cell lines.

In LNCaP cells, the observed decreases in the expression of these G1-S transition-regulators are EVC blueberry juice blend dose-dependent except for CDK2, where a further decrease at 0.8% EVC blueberry juice blend did not reach the significance levels.

In PC-3 cells, the decrease in cyclin and CDK did not reach statistical significance at 0.2-0.4% EVC blueberry juice blend, but became significant at 0.8% EVC blueberry juice blend.

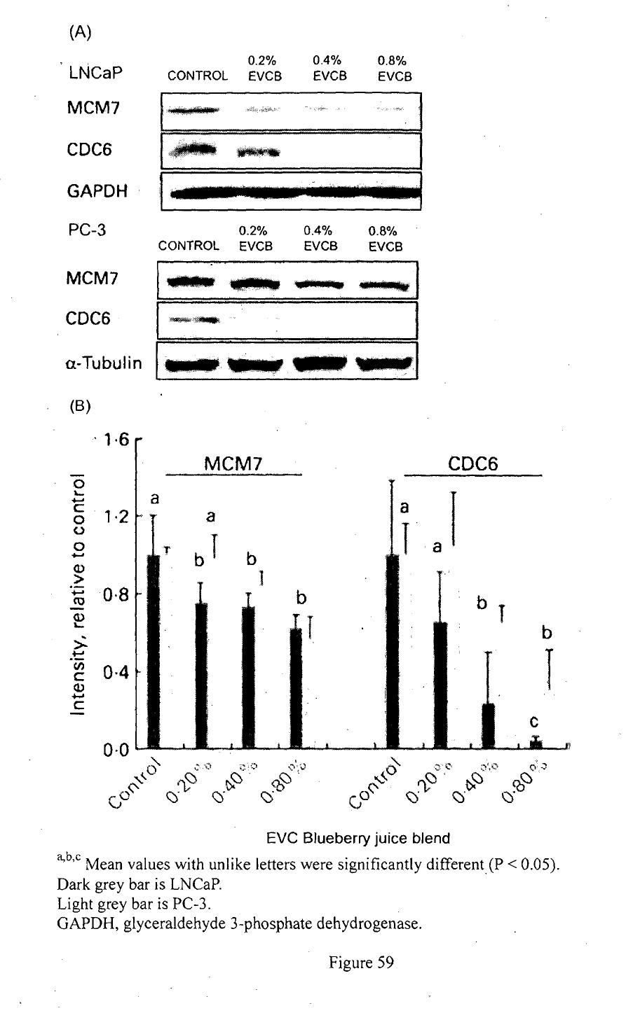

Concurrently, the treatment elevated the protein levels of p27 relative to the control in a dose-dependent manner in LNCaP and although there was a trend of increase in p27 in PC-3, a significant increase was noted only at 0.8% EVC blueberry juice blend (FIG. 59).

The top portion of FIG. 59 shows exponentially growing lymph node-metastasised prostate cancer cells (LNCaP) and bone metastasised androgen receptor negative prostate cells (PC-3) cells that were treated with 0% EVC blueberry juice blend as control (CONTROL) or with the indicated concentrations of EVC blueberry juice blend for 72 h.

Representative immunoblots of cyclin D1, cyclin-dependent kinase (CDK)4, cyclin E, CDK2 and p27 are shown in the lower portion of FIG. 59.

The immuno-labelled proteins on the blots were quantified by densitometry and each protein of interest was normalised by a loading control (α-tubulin or glyceraldehyde 3-phosphate dehydrogenase). The resulting ratio for each dose of EVC blueberry juice blend was then calibrated by the control (0% EVC blueberry juice blend) that was arbitrarily set as 1. Values are means, with standard deviations represented by vertical bars of three independent experiments, noting that mean values were significantly different for PC-3 (light grey bars) compared to those for the controls (P<0.05).

The formation of the pre-replication complex is a pre-requisite for DNA replication. MCM7 and CDC6 are the two major components of the pre-replication complex. As EVC blueberry juice blend inhibited the proliferation in the treated cells, studies were undertaken to determine whether MCM7 and CDC6 levels, and thus cell division cycle 6, were affected by EVC blueberry juice blend treatment.

As shown in FIG. 59, the protein levels of MCM7 and CDC6 in LNCaP and PC-3 cells were reduced by EVC blueberry juice blend treatment. Specifically, in both LNCaP and PC-3 cells, 0.2 or 0.4% EVC blueberry juice blend caused a decrease in the MCM7 and CDC6 levels. A further decrease at 0.8% EVC blueberry juice blend, was noted for the CDC6 levels in LNCaP cells. This finding overall was consistent with the observation that EVC Blueberry juice blend restrains DNA synthesis in prostate cancer cells.

FIG. 59(A) shows immunoblots of Exponentially growing lymph node-metastasised prostate cancer cells (LNCaP) and bone metastasised androgen receptor negative prostate cells (PC-3) cells treated with 0% EVC blueberry juice blend as control (CONTROL) or with the indicated concentrations of EVC blueberry juice blend for 72 hours and then analysed for MCM7 and cell division cycle 6 (CDC6) with immunoblot.

FIG. 59(B) is a graphical representation of immuno-labelled proteins as quantified by densitometry, with each protein of interest being normalised by a loading control. The resulting ratio of each EVC blueberry juice blend concentration was then calibrated by the control (0% EVC blueberry juice blend) that was arbitrarily set to be 1. Values are means, with standard deviations represented by vertical bars of three independent experiments.

It is clear from the foregoing that the invention provides modified polyphenol compounds and compositions comprising the modified polyphenol compounds that address a range of health issues.

The foregoing embodiments are illustrative only of the principles of the invention, and various modifications and changes will readily occur to those skilled in the art. The invention is capable of being practiced and carried out in various ways and in other embodiments. It is also to be understood that the terminology employed herein is for the purpose of description and should not be regarded as limiting.

The term “comprise” and variants of the term such as “comprises” or “comprising” are used herein to denote the inclusion of a stated integer or stated integers but not to exclude any other integer or any other integers, unless in the context or usage an exclusive interpretation of the term is required.

### REFERENCES

Bendele A M, ‘Animal models of rheumatoid arthritis’, J Musculoskel Neuron Interact, 2001; 1(4):377-385
British Pharmacopoeia Commission, ‘British pharmacopoeia’, London, England: Stationery Office (2012)
Calixto J B, Otuki M F and Santos A R S, ‘Anti-inflammatory compounds of plant origin. Part I. Action on arachidonic acid pathway, nitric oxide and nuclear factor κB (NF-κB)’, Planta Med. 2003; 69(11):973-983
Gilmore T D and Herscovitch M, ‘Inhibitors of NF-κB signaling: 785 and counting’, Oncogene, 2006; 25:6887-6899
de Grey A D N J, ‘Life Span Extension Research and Public Debate: Societal Considerations’, Studies in Ethics, Law, and Technology, 2007; 1(1), doi:10.2202/1941-6008.1011
Hiscott J, Kwon H and Génin P, ‘Hostile takeovers: viral appropriation of the NF-κB pathway’, J. Clin. Invest., 2001; 107(2):143-151, doi:10.1172/JCI11918
Miller S C, Huang R, Sakamuru S, Shukla S J, Attene-Ramos M S, Shinn P, Van Leer D, Leister W, Austin C P and Xia M, ‘Identification of known drugs that act as inhibitors of NF-κB signaling and their mechanism of action’, Biochem. Pharmacol. 2010; 79(9):1272-1280
Panchal S K, Ward L and Brown L, ‘Ellagic acid attenuates high-carbohydrate, high-fat diet-induced metabolic syndrome in rats’, Eur. J. Nut., DOI: 10.1007/s00394-012-0358-9, published online 27 Apr. 2012
Quideau S., ‘Chemistry and Biology of Ellagitannins: An Underestimated Class of Bioactive Plant Polyphenols’, World Scientific, 2009
Roman-Blas J A and Jimenez S A, ‘NF-κB as a potential therapeutic target in osteoarthritis and rheumatoid arthritis’, Osteoarthritis Cartilage, 2006; 14(9):839-848
Rowe R C, Sheskey P J, Quinn M E (Ed.), ‘Handbook of Pharmaceutical Excipients’; 6^{th} Ed., London: Pharmaceutical Press (2009).
Sweetman S (Ed.), ‘Martindale: The complete drug reference’, 37^{th} Ed., London: Pharmaceutical Press (2011)
United States Pharmacopeia (USP) and National Formulary (NF), Rockville, Md.: United States Pharmacopeia Convention
http://www.betterhealth.vic.gov.au/bhcv2/bhcarticles.nsf/pages/Arthritis

1. A composition comprising at least one modified polyphenolic compound, wherein the modification comprises nucleophilic substitution of at least one ethoxy moiety to a polyphenolic compound.

2. The composition of claim 1, wherein the at least one modified polyphenolic compound is a modified ellagitannin.

3. The composition of claim 1, wherein the composition is a composition selected from the group consisting of a pharmaceutical composition, a dietary supplement, and a food product.

4-6. (canceled)

7. The composition of claim 3, wherein the food product is selected from the group consisting of ground coffee, black tea leaves, and green tea leaves.

8-9. (canceled)

10. The composition of claim 1, wherein the at least one modified polyphenolic compound is present in an amount of at least 0.5% (w/w).

11. The composition of claim 1, wherein the composition comprises at least three modified polyphenolic compounds.

12-26. (canceled)

27. A method of manufacturing a food product comprising the step of:
combining a modified polyphenolic compound, wherein the modification comprises nucleophilic substitution of at least one ethoxy moiety to a polyphenolic compound, with food, to thereby form the food product.

28. The method of claim 27, wherein the at least one modified polyphenolic compound is a modified ellagitannin.

29. The method of claim 28, wherein the food product is selected from the group consisting of beverages, health bars, confectionary and baked goods.

30. The method of claim 29, wherein the food product is a beverage selected from the group consisting of coffee, black tea and green tea.

31. The method of claim 30, wherein said combining comprises the steps of:
(i) adding a solution comprising the modified polyphenolic compound to ground coffee, black tea leaves or green tea leaves to form a slurry;
(ii) mixing the slurry for a period of 2 to 3 days; and
(iii) drying the slurry.

32-33. (canceled)

34. A method of treating or preventing a medical condition in a mammalian subject, the method comprising the step of administering to the mammalian subject a therapeutically effective amount of a composition comprising at least one modified polyphenolic compound, wherein the modification comprises nucleophilic substitution of at least one ethoxy moiety to a polyphenolic compound.

35. The method of claim 34, wherein the medical condition is selected from the group consisting of aging-related conditions, cancers including metastatic cancers, heart disease, metabolic syndrome, chronic inflammation, acute inflammation, gout, chronic fatigue, obesity, erectile dysfunction, appetite suppression, sleep disorders, urinary tract infections, asthma, enlarged prostate, excessive alcohol consumption, male pattern baldness and arthritis.

36. The method of claim 35, wherein the medical condition is selected from the group consisting of cancers including metastatic cancers, metabolic syndrome, chronic inflammation, acute inflammation, erectile dysfunction and arthritis.

37. The method of claim 36, wherein the medical condition is metabolic syndrome.

38-42. (canceled)

43. The composition of claim 10, wherein the at least one modified polyphenolic compound is a modified ellagitannin.

44. The method of claim 34, wherein the at least one modified polyphenolic compound is a modified ellagitannin.

45. The method of claim 34, wherein the at least one modified polyphenolic compound is present in the composition in an amount of at least 0.5% (w/w).

46. The method of claim 34, wherein the composition comprises at least three modified polyphenolic compounds.
